(19) United States
(12) Patent Application Publication
(10) Pub. No.: US 2026/0000710 A1
(43) Pub. Date: Jan. 1, 2026

(54) Novel Treatments for Cardiovascular Related Disease

(71) Applicant: La Jolla Institute for Immunology, La Jolla, CA (US)

(72) Inventors: Klaus Ley, La Jolla, CA (US); Payel Roy, La Jolla, CA (US); Alessandro Sette, La Jolla, CA (US); John Sidney, La Jolla, CA (US)

(21) Appl. No.: 18/868,365

(22) PCT Filed: Jun. 7, 2023
(86) PCT No.: PCT/US2023/068044

§ 371 Date: Nov. 22, 2024

Related U.S. Application Data
(60) Provisional application No. 63/349,771, filed on Jun. 7, 2022.
(60) Provisional application No. 63/356,964, filed on Jun. 29, 2022.

(51) Int. Cl. A61K 35/17; A61K 40/11; A61K 40/31; A61K 40/32; A61K 40/41; C07K 14/725

(52) U.S. Cl. CPC A61K 35/17; A61K 40/11; A61K 40/31; A61K 40/32; A61K 40/4285; C07K 14/7051; A61K 2239/15; C07K 2317/24; C07K 2317/565

(57) ABSTRACT

Provided herein are engineered T cell receptor (TCR) proteins, nucleic acids, vectors, host cells, methods of treating atherosclerosis-related autoimmune disease, and chimeric antigen receptor expressing T cell (CAR-T) comprising a beta chain CDR3 selected from the amino acid sequence of SEQ ID NOS: 179 to 356 and an alpha chain, wherein the TCR is specific for a human apolipoprotein B (ApoB) epitope, antigen-MHC binding portions, and full-length versions of the same.

### STATEMENT OF FEDERALLY FUNDED RESEARCH

This invention was made with government support under R35 HL145241 awarded by the National Institutes of Health. The government has certain rights in the invention.

### TECHNICAL FIELD OF THE INVENTION

The present invention relates in general to the field of human T cell receptors, and more particularly, to T cell receptors that are specific for human apolipoprotein B for use in treating atherosclerosis-related autoimmune disease.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jun. 7, 2023, is named LJII2020.xml and is 489,560 bytes in size.

### BACKGROUND OF THE INVENTION

Without limiting the scope of the invention, its background is described in connection with cardiovascular disease and/or immune disorders, specifically atherosclerosis-related autoimmune disease.

Atherosclerosis is a pathophysiology that underlies many cardiovascular diseases and is an inflammatory disease of the artery wall with an obligatory secondary autoimmune component¹⁻³. Negative selection of T cells in the thymus is incomplete and autoreactive CD4 and CD8 T cells are detectable in human blood⁴⁻⁶. Studies in transgenic mice that modeled tissue-specific expression patterns of endogenous antigens, demonstrated that autoreactive T cell responses against certain tissue-restricted self-antigens, unlike those targeting ubiquitously expressed proteins, are comparable to T cell responses to foreign antigen^{7,8}.

Recent studies have highlighted the involvement of an autoantigen-specific adaptive immune network in atherosclerosis³. Antigen-specific and MHC Class-II-restricted productive recall interactions between antigen-experienced CD4⁺ T cells and antigen presenting cells (APCs) occur in the aortic walls of atherosclerotic mice⁹. Oligoclonal proliferation and expression of activation markers were observed in plaque-associated T cells, indicating that human T cells respond to unknown antigens in the vessel wall^{10,11}.

The best evidence for a relevant atherosclerosis antigen supports a major role for low-density lipoprotein (LDL) and its core protein Apolipoprotein B (APOB)¹²⁻¹⁶. Both humans and mice have circulating antibodies to native or modified LDL and APOB. While the antibodies against modified lipid moieties of LDL are predominantly of the immunoglobulin M (IgM) isotype, most antibodies to the protein component APOB are of the IgG isotype¹⁷⁻¹⁹. To produce IgG, B cells must undergo isotype switching, which requires help from activated CD4⁺ follicular helper T cells²⁰. Thus, the existence of IgG antibodies to human APOB indicates that APOB-specific human CD4⁺ T cells exist in vivo.

Peptide presentation on heterodimeric Human Leukocyte Antigen (HLA) Class II molecules (encoded by DP, DQ and DR alpha and beta chain genes)²¹ engages epitope-specific αβ T Cell Receptors (TCRs). Human HLA-II genes are highly polymorphic, with most numbers of allelic variants being reported for DP, DQ, DR beta chain (B) genes²¹. The αβTCR clonotypic repertoire is variable²² and predominantly consists of individual-specific private TCRs²³. Based on immunogenicity studies in other autoimmune-related inflammatory diseases^{4,24,25}, only specific TCR-HLA-II-epitope trimolecular combinations trigger CD4⁺ T cell activation²⁶. For this reason, it is important to resolve antigen-specific responses to single epitopes in HLA-typed donors and identify antigenic candidates that elicit strong CD4⁺ T activation in a large fraction of individuals²⁷.

Early attempts to elucidate the identities of human APOB-specific CD4⁺ T cells involved characterization of in vitro stimulated splenocytes and T cell hybridomas generated from oxidized-LDL immunized human ApoB100 (huB100tg) transgenic mice^{28,29}. These mice revealed preferential VDJ usage of antigen-specific mouse CD4⁺ T cell clones. However, APOB-responsive human CD4⁺ T cells remained unidentified. Using a DRB1*07:01 Class-II tetramer molecule, loaded with human APOB_{3036_3050}, the inventors previously provided the first evidence that APOB epitope-specific CD4⁺ T cells exist in human peripheral blood mononuclear cells (PBMCs)¹. However, because an individual tetramer can screen only one HLA-II-peptide combination at a time, this study could not assess antigenicity of APOB₃₀₃₆₋₃₀₅₀ in the context of other HLA-II alleles or in comparison to other peptides.

Despite these advances, a need remains for novel agents and methods for use in treating atherosclerosis-related autoimmune disease.

### SUMMARY OF THE INVENTION

As embodied and broadly described herein, an aspect of the present disclosure relates to an engineered T cell receptor (TCR) comprising a human T cell beta chain with a CDR3 selected from the amino acid sequence of SEQ ID NOS: 179 to 356 and an alpha chain, wherein the TCR is specific for a human apolipoprotein B (ApoB) epitope. In one aspect, the engineered TCR binds to a Class II HLA and a peptide selected from SEQ ID NOS: 358, 360, 361, 367, 368, or 372. In another aspect, the TCR comprises a beta chain CDR3 having at least 95, 96, 97, 98, or 99% identity to the amino acid sequence of SEQ ID NOS: 179 to 356. In another aspect, the TCR is humanized. In another aspect, the TCR comprises a beta chain having at least 90, 91, 92, 93, 94, 95, 96, 97, 98, 99, or 100% identity to the nucleotide sequence of SEQ ID NO: 1. In another aspect, the TCR is further defined as a soluble TCR, wherein the soluble TCR does not comprise a transmembrane domain, or comprises a transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the TCR further comprising a detectable label. In another aspect, the TCR is covalently bound to a therapeutic agent, an immunotoxin, or a chemotherapeutic agent. In another aspect, the beta chain CDR3 is selected from SEQ ID NO: 179-232. In another aspect, the TCR is part of a multivalent TCR complex comprising a plurality of TCRs described herein above. In another aspect, the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule; or the human apolipoprotein B (ApoB) epitope is an immunodominant epitope.

As embodied and broadly described herein, an aspect of the present disclosure relates to a polypeptide encoding the TCR described herein above. As embodied and broadly described herein, an aspect of the present disclosure relates to a polynucleotide encoding the polypeptide described herein above. As embodied and broadly described herein, an aspect of the present disclosure relates to an expression vector encoding the TCR described herein above. In one aspect, the sequence encoding the TCR is under the control of a promoter. In another aspect, the expression vector is a viral or a retroviral vector. In another aspect, the vector further encodes a linker domain positioned between the alpha chain and beta chain. In another aspect, the linker domain comprises one or more protease cleavage sites, or wherein the one or more cleavage sites are separated by a spacer.

As embodied and broadly described herein, an aspect of the present disclosure relates to a host cell engineered to express the TCR described herein above. In one aspect, the cell is a Treg cell, mesenchymal stem cell (MSC), or induced pluripotent stem (iPS) cell. In another aspect, the host cell is an immune cell. In another aspect, the host cell is a T cell that is a CD4⁺ T cell or γδ T cell. In another aspect, the host cell is a regulatory T cell (Treg) selected from a follicular regulatory T cell, a ST2 T reg, an activated T reg, or an effector Treg. In another aspect, the host cell is autologous or allogeneic.

As embodied and broadly described herein, an aspect of the present disclosure relates to a method for engineering a host cell comprising contacting an immune cell with the TCR or the expression vector described herein above. In another aspect, contacting is further defined as transfecting or transducing, wherein transfecting comprises electroporating RNA encoding the TCR described herein above into the immune cell.

As embodied and broadly described herein, an aspect of the present disclosure relates to a method for treating a subject with atherosclerosis with autoimmunity to human apolipoprotein B (APOB) peptides, the method comprising: administering to the subject an effective amount of one or more immune cells modified by cloning genes of the alpha and beta chains of a T cell receptor (TCR) ex vivo to express a chimeric antigen receptor specific for the APOB peptide, wherein the chimeric antigen receptor comprises an alpha chain CDR3 and a beta chain CDR3 having the amino acid sequence of SEQ ID NO: 179 to 356. In one aspect, the immune cell is regulatory T cell (Treg) selected from a follicular regulatory T cell, a ST2 T reg, an activated T reg, or an effector Treg, mesenchymal stem cell (MSC), or induced pluripotent stem (iPS) cell, or a peripheral blood lymphocyte. In another aspect, the method further comprises at least one of: sorting the immune cells into T cells to isolate TCR engineered T cells; performing a T cell cloning of the immune cells by serial dilution; or expanding a T cell clone from the immune cells by a rapid expansion protocol. In another aspect, the subject is identified to have an HLA selected from:

| DPB1 | DQB1 | DRB1 | DRB3 | DRB4 | DRB5 |
|---|---|---|---|---|---|
| 04:01 + 11:01 | 03:03 + 05:01 | 09:01 + 10:01 | NP + NP | 01:01 + NP | NP + NP |
| 04:01 + 13:01 | 06:02 + 06:03 | 13:01 + 15:01 | 01:01 + NP | NP + NP | 01:01 + NP |
| 04:01 + 04:01 | 03:01 + 03:02 | 04:01 + 11:01 | 02:02 + NP | 01:01 + NP | NP + NP |
| 04:01 + 05:01 | 02:02 + 03:03 | 07:01 + 09:01 | NP + NP | 01:01 + 01:01 | NP + NP |
| 02:02 + 04:02 | 02:01 + 03:01 | 03:01 + 11:02 | 02:02 + 02:02 | NP + NP | NP + NP |
| 03:01 + 04:01 | 03:03 + 06:02 | 09:01 + 15:01 | NP + NP | 01:01 + NP | 01:01 + NP. |

In another aspect, the the immune cell is a T cell selected from a regulatory T cell (Treg) selected from a follicular regulatory T cell, a ST2 T reg, an activated T reg, or an effector Treg. In another aspect, the TCR engineered cells are autologous or allogeneic. In another aspect, the method further comprises administering a second therapy selected from immunotherapy, surgery, or biotherapy. In another aspect, the one or more immune cells are administered intravenously, intraperitoneally, intratracheally, intramuscularly, endoscopically, intralesionally, percutaneously, subcutaneously, regionally, or by direct injection or perfusion.

As embodied and broadly described herein, an aspect of the present disclosure relates to a chimeric antigen receptor expressing T cell (CAR-T) comprising an antigen recognition moiety and a T-cell activation moiety, wherein the T-cell activation moiety comprises a transmembrane domain, and wherein the antigen recognition moiety comprises a T cell receptor beta chain CDR3 is selected from the amino acid sequence of SEQ ID NOS: 179 to 356 and an alpha chain, wherein the TCR is specific for a human apolipoprotein B (ApoB) epitope. In one aspect, the TCR binds to a Class II HLA and a peptide selected from SEQ ID NOS: 358, 360, 361, 367, 368, or 372. In another aspect, the transmembrane domain is a CD28 transmembrane domain or a CD8a transmembrane domain. In another aspect, the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the TCR comprises a beta chain having at least 90, 91, 92, 93, 94, 95, 96, 97, 98, 99, or 100% identity to the nucleotide sequence of SEQ ID NO: 1. In another aspect, the antigen recognition moiety comprises a beta chain CDR3 having an amino acid sequence selected from one of SEQ ID NO: 179-232. In another aspect, the immune cell is a T cell selected from a regulatory T cell (Treg) selected from a follicular regulatory T cell, a ST2 T reg, an activated T reg, or an effector Treg.

### BRIEF DESCRIPTION OF THE DRAWINGS

For a more complete understanding of the features and advantages of the present invention, reference is now made to the detailed description of the invention along with the accompanying figures and in which:

FIGS. 1A to 1L: In vitro expanded antigen-specific T cells respond specifically to cognate peptides upon subsequent re-stimulation. Human PBMCs were expanded for 14 days with APOB₂₀ or CEFX-II pool and then re-stimulated with the cognate or the irrelevant peptide pools. Antigen-induced T cell responses were monitored using IFNγ ELISpot assay (FIG. 1A and FIG. 1B) or FACS-based intracellular cytokine staining for TNF (FIG. 1C, FIG. 1G), IFNγ (FIG. 1D, FIG. 1H), IL-4 (FIG. 1E, FIG. 1I) and IL-17A (FIG. 1F, FIG. 1J) producing CD4⁺ T cells. FIG. 1A) Representative images from an IFNγ ELISpot assay after 24 h of re-stimulation. PHA-L: positive control. FIG. 1B) Numbers of IFNγ-secreting cells in stimulated sets minus the background signal in unstimulated controls, expressed as spot forming cells (SFCs) per 10⁶ PBMCs. FIG. 1C-1F) Representative FACS plots gated on singlets, live, dump⁻, CD3⁺, CD8⁻, CD4⁺ T cells, showing % CD40L⁺ cytokine⁺ populations after 6 h of re-stimulation. Large dots: IL-17A (F). FIG. 1G-1J) Background subtracted frequencies of % CD40L⁺ cytokine⁺ CD4⁺ T cells in cognate or irrelevant pool stimulated PBMCs. Bars show mean with standard error of mean (SEM). Statistical comparisons between two different stimulation conditions (FIG. 1B, FIG. 1G-1J) were performed using Mann-Whitney test. **p<0.01.

FIGS. 2A to 2D: Activation-induced surface expression of CD40L and CD69 (AIM assay) allows sensitive detection of APOB-specific CD4⁺ T cells in ex vivo stimulated PBMCs. FIG. 2A) % CD40L⁺CD69⁺ (AIM⁺) CD4⁺ T cells in paired sets of APOB₂₀ stimulated and unstimulated PBMCs. FIG. 2B) Representative FACS plots and FIG. 2C) Violin plots showing median frequencies of % CD40L⁺CD69⁺ (AIM⁺) CD4⁺ T cells in unstimulated and 24 h Actin or APOB₂₀ or CEFX-II stimulated PBMCs. FIG. 2D) Average stimulation indices (% CD40L⁺CD69⁺ CD4 T cells in stimulated sets over those in unstimulated control) for Actin, APOB₂₀ and CEFX-II peptide pools. Pairwise statistical comparisons (FIG. 2A) were performed with the Wilcoxon test. Statistical tests for mean response across different stimulation conditions and unstimulated sets (FIG. 2C, FIG. 2D) were performed using one-way ANOVA and Tukey's multiple comparison test. **p<0.01, ***p<0.001, ****p<0.0001.

FIGS. 3A to 3F: Deconvolution of T cell responses to APOB₂₀ peptide pool identifies dominant antigenic sites in APOB. Human PBMCs were expanded with APOB₂₀ peptide pool and then re-stimulated with each peptide individually. Peptide-specific responses in PBMCs from 19 HLA-typed donors were evaluated using IFNγ ELISpot assay. FIG. 3A) Representative images from IFNγ ELISpot assay. PHA-L: positive control. FIG. 3B) Numbers of donors (X-axis) that respond to each peptide (Y-axis). FIG. 3C) Sequences and start sites of the dominant epitopes (SEQ ID NOS: 358, 360, 361, 367, 368, or 372). FIG. 3D) Average responses (spot forming cells, SFC) to individual peptides P2, 4, 5, 11, 12 or 17 and to the other 14 peptides. FIG. 3E) Representative images of ELISpot wells and quantification showing IFNγ responses in PBMCs expanded and re-stimulated with a pool of dominant epitopes (APOB₆), either alone or in the presence of pan-HLA-I or pan-HLA-II (DP+DQ+DR) blocking antibodies. FIG. 3F) % TNF⁺IFNγ⁺, % TNF⁺IFNγ⁻ and % TNF⁻IFNγ⁺ populations among CD4⁺ and CD8⁺ T cell subsets are shown for each peptide. Pink—CD4, yellow—CD8. Data from 10 donors. Bars represent mean with SEM. Statistical tests for peptide-specific responder frequencies (FIG. 3B) were performed using Fisher's exact test. Comparison of response magnitudes across peptides (FIG. 3D) or effect of blocking antibodies (FIG. 3E) were examined using Kruskal-Wallis test. FIG. 3 Red: dominant epitopes, purple: remaining peptides (B,D). *p<0.05, **p<0.01, ***p<0.001, ****p<0.0001.

FIGS. 4A to 4H 4: APOB-derived dominant epitopes trigger expansion and activation of an oligoclonal population of CD4⁺ T cells in human PBMCs. FIG. 4A) Total and unique counts of productive (in-frame for protein translation) TCR templates identified in control AIM⁻ and APOB₆-specific AIM⁺ CD4⁺ T cells. Y-axis: counts×10⁴. FIG. 4B) Box-plots showing Simpson's clonality index measured for AIM⁻ and AIM⁺ productive TCRs. FIG. 4C) Comparison of repertoire overlap between AIM⁺ productive TCRs and those from naïve or TCM or TEM subsets. Values denote the Jaccard index, a statistical measurement of similarity between sample sets. Rows represent data from individual donors. FIG. 4D) Matrix showing TCR repertoire overlap across donor-specific AIM⁺ productive TCRs. FIG. 4E) Total numbers of unique productive TCR clones identified in AIM⁺ CD4⁺ T cells from individual donors (rows) and are present within a specific range of copy numbers (columns). FIG. 4F) Red bars and values showing the % frequencies of the top 10 most abundant rearrangements of all productive TCRs detected in AIM⁺ cells in each donor. Grey shaded areas represent the combined frequencies of all other clones. FIG. 4G) Bars represent template copy numbers of the top-expanded clone in different sorted CD4⁺ T populations. Total numbers of productive TCR templates in AIM⁻ and AIM⁺ subsets (Day 14) and in naïve, TCM and TEM lineages (Day 0) are also indicated. FIG. 4H) Nucleotide sequence (SEQ ID NO:1 nucleic acids 1 to 88m) and bio-identity of the top-ranked APOB6-specific TCR rearrangement (SEQ ID NO:179). Details of the rearrangement are colored coded by component. Statistical comparisons between total and unique TCR counts (FIG. 4A) and Simpson's clonality in AIM⁻ and AIM⁺ populations (FIG. 4B) were performed using Mann-Whitney test. Statistical comparisons of specific TCR clonal frequencies in AIM⁻ vs AIM⁺ cells and in naïve vs TCM or naïve vs TEM populations (FIG. 4G) were performed using Fisher's exact test. **p<0.01, ****p<0.0001.

FIGS. 5A to 5C: Ex vivo activated APOB₆-specific CD4⁺ T cells are enriched in memory T cell markers. PBMCs were stimulated for 24 h with APOB₆ peptide pool and surface expression of activation markers on CD4⁺ T cells was assessed by flow cytometry. FIG. 5A) Representative FACS plots in two donors showing APOB₆-induced expressions of three different combinations of T cell activation markers, as assessed in a sequential gating scheme. Top row: AIM1% CD40L⁺CD69⁺; middle row: AIM2% CD25⁺4-1BB⁺; bottom row: AIM3% CD25⁺OX-40⁺. % AIM⁺CD4⁺ T represent sum of AIM1, AIM2 and AIM3 frequencies. % AIM⁻CD4⁺ T represent those excluded from all three AIM combinations. FIG. 5B) Representative FACS plots in two donors showing CD45RA⁺CCR7⁺ naïve, CD45RA⁻CCR7⁺ central memory (TCM) and CD45RA⁻CCR7⁻ effector memory (TEM) populations in AIM1+, AIM2⁺, AIM3⁺, AIM⁻, and all CD4⁺ T cells. FIG. 5C) Median frequencies of naïve and memory (TCM+TEM) fractions within AIM⁺, AIM⁻ and all CD4⁺ T subsets in APOB₆ stimulated PBMCs. To calculate these fractions for the AIM⁺ subset, the numbers of cells expressing either naïve or memory markers were first counted within AIM1⁺, AIM2⁺ and AIM3⁺CD4⁺ T cells individually and then their sum was divided by the total number of AIM⁺ (AIM1+AIM2+AIM3) CD4⁺ T cells in that donor. Statistical comparisons across different subsets (FIG. 5C) were performed using Kruskal-Wallis test. ***p<0.001, ****p<0.0001.

FIGS. 6A to 6F: Dominant APOB epitopes elicit diverse set of T helper cytokine responses in ex vivo stimulated PBMCs. FIG. 6A) The 20 APOB peptides were ranked by their respective strength of elicited responses (from FIG. 3). Ranked epitopes (X-axis) were plotted against their relative contributions to the cumulative aggregate response detected in all donors (Y-axis). Dominant “pos peps” APOB peptides P2,4,5,11,12,17 contributed >60% (dotted red line) to the cumulative response and had responder frequency >50%. APOB peptides P3,7,16,18 that contribute <5% (dotted blue line) to the cumulative response and had responder frequencies ≤10% were selected as a contrasting set of “neg peps”. FIG. 6B) Median frequencies of % AIM⁺CD4⁺ T cells and C) Median secreted levels (pg/ml) of IL-2, TNF, IFNγ, IL-17A and IL-10, upon 24 h stimulation with “neg” or “pos” APOB peptide pools or with CEFX-II pool. FIG. 6D) Lipoprotein(a), lipid profile, hsCRP and HbA1c levels in the blood of group 1 and 2 donors on the day of sample collection. FIG. 6E) Median levels (mg/dL) of total (left) and non-HDL (right) cholesterol in blood. FIG. 6F) Median levels (pg/ml) of TNF secreted in response to 24 h stimulation with APOB₆ (pos peps) pool. Y-axis (FIG. 6C, FIG. 6F) log₁₀ transformed and data points with 0 or negative values collapsed onto the minimum value on the scale. Statistical comparisons across peptide pools (FIG. 6B, FIG. 6C) were performed using Kruskal-Wallis test. Statistical tests between the two donors groups (FIG. 6E, FIG. 6F) were done using Mann-Whitney test. *p<0.05, **p<0.01, ****p<0.0001.

FIGS. 7A to 7D: Dominant APOB epitopes trigger increased CD4⁺ T activation and augmented secretion of proinflammatory cytokines in patients with more severe CAD. PBMCs from matched clinical samples from patients with low and high disease severity were stimulated with APOB₆ for 24 h. CD4⁺ T cell activation and naïve vs memory marker expression was examined using the AIM assay. Secreted T helper cytokines were profiled using CBA. FIG. 7A) Representative FACS plots showing frequencies of AIM⁺CD4⁺ T cells (AIM1,2 and 3 in the serial gating scheme) in unstimulated and APOB₆ stimulated PBMCs. FIG. 7B) Median Gensini scores and frequencies of AIM⁺CD4⁺ T cells (sum of % AIM1,2,3). FIG. 7C) Median frequencies of memory (TCM+TEM) fractions in AIM⁺CD4⁺ T, calculated as detailed in the legend of FIG. 5C. FIG. 7D) Median secreted levels (pg/ml) of induced IFNγ, TNF and IL-10. Y-axis (FIG. 7D) log₁₀ transformed and data points with 0 or negative values collapsed onto the minimum value on the scale. Statistical comparisons between the two groups of patients (FIG. 7B-FIG. 7D) were performed using Mann-Whitney test. *p<0.05, **p<0.01, ****p<0.0001.

FIGS. 8A to 8C: Broadly binding MHC Class-II-restricted immunodominant epitopes in human APOB allow phenotypic evaluation of atherosclerosis-related CD4⁺ T responses and enabled identification of APOB-specific autoreactive TCR clones. FIG. 8A) Human HLA-II binder alleles expressed in donor samples are shown in the antigen presenting cell (left). Each allele is color coded by the corresponding color of the APOB epitopes (middle, with sequence and position in APOB) that it binds. Bio-identities of clones whose AIM⁺ vs AIM⁻ log₁₀ odds ratios are >1 and FDR corrected Fisher's exact test p values <10⁻²⁰⁰ are shown in the CD4⁺ T cell. Sequences are color coded by component. Resolved V gene green, translated CDR3 region (which includes start of CDR3 in V gene green, N1 junction purple, D-gene blue, N2 junction pink, end of CDR3 in J gene orange) and resolved J-gene orange are shown (SEQ ID NOS: 179, 224, 225, 180, 181, 183, 184, 214, 342, 190, 191, 193, 194, 195). FIG. 8B) Stimulation of human PBMCs with a pool of dominant epitopes (APOB₆) elicits memory CD4⁺ T responses and triggers secretion of multiple T helper cytokines. FIG. 8C) Higher frequencies of antigen-experienced APOB₆-reactive CD4⁺ T cells and increased proinflammatory cytokine responses are observed in patients with more severe coronary artery disease.

FIGS. 9A to 9D: MHC-II binding of APOB₂₀ peptides and cytokine analysis in expansion-based re-stimulation assays. FIG. 9A) Binding affinities (nM), as assessed in a competition binding assay, of in silico predicted peptides to a reference set of most common human HLA Class II alleles. Non-binding: affinities >1000 nM. FIG. 9B) Schematic workflow for antigen-dependent expansion of PBMCs and subsequent re-stimulation-based detection of cytokine producing antigen-specific T cells. FIG. 9C) and FIG. 9D) PBMCs were expanded with APOB₂₀ pool and cytokine responses in 6 h APOB₂₀ restimulated and in unstimulated control cells were assessed using FACS-based ICS assay. Cytokine producing cells were gated on singlets, live, dump⁻, CD3⁺, CD8⁻, CD4⁺, CD40L⁺ T cells. FIG. 9C) % CD40L⁺ cytokine⁺CD4⁺ T cells in paired sets of APOB₂₀ restimulated and unstimulated PBMCs. Th cytokines TNF, IFNγ, IL-4, IL-17A and IL-10 responses are shown. FIG. 9D) Median frequencies of APOB₂₀-induced Th1 (IFNγ), Th2 (IL-4), Th17 (IL-17A), Treg or Tr1 (IL-10) production in CD40L⁺CD4⁺ activated T cells. Y-axis (FIG. 9C and FIG. 9D) log 10 transformed (data points with 0 or negative values collapsed onto the minimum value on the scale). Pairwise statistical comparisons (FIG. 9C) were performed with the Wilcoxon test. Statistical tests across multiple cytokine responses (FIG. 9D) were performed using Kruskal-Wallis test. **p<0.01, ****p<0.0001.

FIGS. 10A to 10D: Workflow for AIM assay. FIG. 10A) Schematic workflow for the 24 h AIM assay. FIG. 10B) Example of flow cytometry gating strategy to designate CD4⁺ T cells for AIM marker evaluation in ex vivo stimulated PBMCs. FIG. 10C) Representative FACS plots showing % CD40L⁺CD69⁺+(AIM⁺) CD4⁺ T cells after 6 h and 24 h of stimulation with APOB₂₀ or CEFX-II pools. FIG. 10D) Normalized values of background subtracted % CD40L⁺CD69⁺ (AIM⁺) CD4⁺ T cells in PBMCs stimulated under conditions of no HLA-II block and pan HLA-II blocking.

FIGS. 11A to 11C: Deconvolution and IFNγ responses to dominant epitopes using original and scrambled peptide sequences. FIG. 11A) Schematic workflow for deconvolution of responses from APOB₂₀ pool to individual peptides. FIG. 11B) Amino acid sequences of original and scrambled versions of the six dominant APOB peptides (SEQ ID NOS: 358, 360, 361, 367, 368, r 372, and SEQ ID NOS:378, 379, 380, 381, 382, 383). FIG. 11C) Representative ELISpot wells showing IFNγ responses in PBMCs restimulated with either the original or the scrambled APOB peptides (left). Unstimulated negative controls and APOB₂₀ pool-stimulated positive control PBMCs are shown (right).

FIGS. 12A to 12E: Validation of responses to dominant epitopes using flow cytometry-based assays. FIG. 12A) Schematic workflow for expansion and subsequent re-stimulation with individual peptides. Cytokine responses were evaluated using flow cytometry-based ICS assay. FIG. 12B) Cytokine producing cells were gated on singlets, live, dump⁻, CD3⁺, CD4⁺ or CD8⁺ T cells. Representative FACS plot showing % TNF⁺IFNγ⁺, % TNF⁺IFNγ⁻ and % TNF IFNγ populations among CD4^{+ and CD}8⁺ T cell subsets in unstimulated and individual peptide stimulated PBMCs. FIG. 12C) Box plots showing median frequencies of Live CD3⁺ T cells present in PBMCs expanded and re-stimulated with individual epitopes. Live cells were gated on CD3⁺ T cells that stained negative for the fixable live/dead dye. FIG. 12D) Each epitope-specific expanded PBMC set was re-stimulated with the cognate peptide or an irrelevant negative control peptide. Cytokine producing cells were gated on singlets, live, dump⁻, CD3⁺, CD8⁻, CD4⁺, CD40L⁺ T cells. Background (responses in unstimulated set) subtracted frequencies of % CD40L⁺ cytokine⁺CD4⁺ T cells in cognate and irrelevant peptide stimulated sets are shown for TNF and IFNγ. E) % CD40L⁺CD69⁺ (AIM⁺) CD4⁺ T cells in unstimulated and Actin (JPT) or APOB₆ (JPT) or CEFX-II (JPT) pool stimulated PBMCs. CD4⁺ T cells were gated on singlets, live, dump, CD3 cells. Bars represent mean values with standard error of mean (SEM) shown. Statistical tests between mean responses to cognate and irrelevant peptide (FIG. 12D) were done using Mann-Whitney test. Average responses to different peptide pools were compared to unstimulated controls (FIG. 12E) using the Kruskal-Wallis test. *p<0.05, ****p<0.0001.

FIGS. 13A to 13C. Determining associations between donor HLA-II expression, peptide-HLA binding affinities and immunogenicity of APOB epitopes. FIG. 13A) PBMCs from two donors were expanded with APOB peptide P2. Left panel shows the binding affinities of donor HLA-DR alleles (at loci DRB1 and DRB3/4/5) for P2, as determined in our competition binding assay. Middle panel shows IFNγ responses in PBMCs expanded and restimulated with P2, either alone or in the presence of a pan-HLA-DR blocking antibody. Right panel is a FACS plot showing surface expression of HLA-DR on live CD3⁻ CD19⁺ B cells, the primary antigen presenting cells in PBMCs harvested after P2-dependent expansion. HLA-DR FMO (fluorescence minus one) was used as background control. FIG. 13B) Left panel shows FACS plots for surface expression of HLA-DP and HLA-DR molecules on single HLA-II transfected B cell lines expressing either HLA DPB1*05:01 (blue) or HLA DRB3*02:02 (purple) alleles. Right panel shows binding affinities of two sets of contrasting peptides for these HLA-II alleles. FIG. 13C) Immunogenicity of non-binder (left) and binder (right) peptide-HLA-II combinations was assessed in IFNγ ELISpot assay in donors that expressed the allele under study (upper panel: DPB1*05:01, bottom panel: DRB3*02:02). Peptide-expanded PBMCs were incubated with single HLA-II transfected cell lines that were pre-loaded with the cognate peptide. No peptide loaded cell lines were used as background controls. Unstimulated and exogenous cognate peptide treated PBMC alone sets served as negative and positive controls, respectively.

FIGS. 14A and 14B Immunogenicity of dominant APOB epitopes and expression of binder HLA-II alleles examined in individual donors. FIG. 14A) Breadth of response denoted by the total number of epitopes identified by each donor. Red shading in the bars represent numbers of dominant epitopes (P2, 4, 5, 11, 12 or 17) recognized per donor. FIG. 14B) Peptide-specific heatmaps showing expressions of binder alleles (rows) in HLA-typed donor (columns) samples examined in the epitope-specific deconvolution assay (FIG. 3). White, light purple and dark blue colored boxes denote 0, 1 or 2 copies of each allele, respectively.

FIGS. 15A to 15F: Schematic, gating strategy and analysis of APOB₆-specific TCRβ repertoire. FIG. 15A) Schematic workflow for cell isolation and TCR sequencing. FIG. 15B) Example of flow cytometry gating strategy to isolate activated AIM⁺ and control AIM⁻ CD4⁺ T cells from PBMCs that were expanded and restimulated for 24 h with APOB₆ pool (P2,4,5,11,12,17). APOB₆ expanded but unstimulated sets were used to set the gates for activation marker expression. FIG. 15C) Example of flow cytometry gating strategy to isolate naïve, central memory (TCM) and effector memory (TEM) CD4⁺ T cells from Day 0 samples. CD4⁺ T cells (in B and C) were gated on singlets, live, dump⁻, CD3⁺ cells. FIG. 15D) Simpson's clonality measured for productive TCRs from AIM⁻, AIM⁺, naïve, TCM and TEM CD4⁺ T subsets. Horizontal bars in box-plots represent median values and whiskers denote min to max data distribution. FIG. 15E) Matrix showing Venn diagram analysis of productive TCR clonotypes shared amongst AIM⁺ CD4⁺ T cells from six donor PBMCs. FIG. 15F) Copy numbers of six productive AIM⁺ TCR clonotypes that are shared between donors 2 and 5 and are present in >10 copies in both donors. Statistical comparison of clonality across all subsets (FIG. 15D) was done using one-way ANOVA and Dunnett's multiple comparison tests. *p<0.05, ***p<0.001.

FIGS. 16A and 16B: Sequential gating strategy to assess expression of multiple combinations of T cell activation-induced markers (AIM) on peptide stimulated CD4⁺ T cells. FIG. 16A) Representative FACS plot showing APOB₆-induced % AIM⁺CD4⁺ T cells gated using sequential gating scheme in a 24 h AIM assay. CD4⁺ T cells were gated on singlets, live, dump⁻, CD3⁺ cells. Gates for activation markers were set using unstimulated negative controls. Left, % CD69⁺CD40L⁺ (AIM1), gated on CD4⁺ T; middle, % CD25⁺4-1BB⁺ (AIM2), gated on AIM1⁻CD4⁺ T; right, % CD25⁺OX-40⁺ (AIM3), gated on AIM⁻ 2CD4⁺ T. AIM⁻ cells did not express any combination of the analyzed AIM markers (AIM1,2,3⁻). FIG. 16B) Median frequencies of naïve and memory (TCM+TEM) fractions within AIM⁺, AIM⁻ and all CD4⁺ T subsets in CEFX-II stimulated PBMCs. Statistical comparisons across different subsets (B) were performed using Kruskal-Wallis test. ****p<0.0001.

FIGS. 17A to 17C: Dominant APOB epitopes trigger stronger CD4⁺ T activation and proliferation, as compared to APOB peptides with low reactivity. FIG. 17A) Proliferation of CD4⁺ T cells in response to APOB “neg” and “pos” peps were compared in a CFSE dilution assay. Left, gates were set on CFSE low CD4⁺ T cells using unstimulated controls with and without CFSE staining. Middle, representative FACS plots showing frequencies of CFSE low CD4⁺ T cells in “neg” and “pos” peps stimulated PBMCs. Unstimulated negative controls and soluble CD3/CD28 stimulated positive controls are shown. Right, % CFSE low CD4⁺ T cells (mean with SEM shown). CD4⁺ T cells were gated on singlets, live, dump⁻, CD3⁺ cells. FIG. 17B) Representative FACS plots showing frequencies of % AIM⁺ CD4⁺ T cells (AIM1, 2 and 3 in the serial gating scheme). PBMCs were stimulated for 24 h with “neg” or “pos” APOB peptide pools or with CEFX-II pool. FIG. 17C) Median levels (pg/ml) of IL-2, IFNγ, IL-17A and IL-10 secreted in response to 24 h stimulation with the APOB₆ pool. Y-axis (C) log 10 transformed and data points with 0 or negative values collapsed onto the minimum value on the scale. Statistical comparison of responses between two APOB pools (FIG. 17A) and between two donor groups (FIG. 17C) were performed using Mann-Whitney test. *p<0.05.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “Antigen” or “Ag” refer to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycoprotein, polynucleotide, polysaccharide, lipid or the like. An antigen can be synthesized, produced recombinantly, or derived from a biological sample. Biological samples that contain antigens can include tissue samples, cardiac samples, cells, biological fluids, or combinations thereof. Antigens can be produced by cells that have been modified or genetically engineered to express an antigen. The antigen herein is apolipoprotein B (ApoB).

As used herein, the terms “epitope” or “antigenic epitope” refer to any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three-dimensional structural characteristics, as well as specific charge characteristics.

As used herein, the term “antigen processing” refers to the processing of a protein into peptides for presentation by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8^{th} Ed.) 2011 Garland Science, NY; chapters 6, 9 and 16, relevant portions incorporated herein by reference). For example, processed antigen peptides originating in the cytosol (e.g., APOB) are generally from about 7 amino acids to about 11 amino acids in length and will associate with class I MEW molecules, whereas peptides processed in the vesicular system (e.g., bacterial, viral) will generally vary in length from about 10 amino acids to about 25 amino acids and associate with class II MHC molecules.

As used herein, the term “binding domain” (also referred to as a “binding region” or “binding moiety”), refers to a molecule or portion thereof (e.g., peptide, oligopeptide, polypeptide, protein) that possesses the ability to specifically and non-covalently associate, unite, or combine with a target (e.g., apolipoprotein B (ApoB)). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv), receptor ectodomains, ligands (e.g., cytokines, chemokines), or synthetic polypeptides selected for their specific ability to bind to a biological molecule, a molecular complex or other target of interest.

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs may be codon optimized to enhance expression in a particular host cell, such as T cells (Scholten et al., Clin. Immunol. 119:135, 2006, relevant portions incorporated herein by reference).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, relevant portions incorporated herein by reference). Assays for assessing affinity or apparent affinity or relative affinity are also known.

As used herein, the term “chimeric antigen receptors (CARs),” refers to artificial T cell receptors, chimeric T cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to APOB. In some embodiments, CARs comprise an intracellular activation domain, a transmembrane domain, and an extracellular domain comprising the beta chain CDR3 of the present invention that binds to the APOB peptide in the contect of Class II MHC. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

As used herein, the term “chimeric antigen receptor T cell” or “CAR-T” refers to a T cell that has been modified to express the TCR of the present disclosure. Non-limiting examples of T cells that can be made into CAR-T cells include: autologous or allogeneic T cells, or even, regulatory T cells, CD4⁺ T cells, CD8⁺ T cells, gamma-delta T cells, NK cells, invariant NK cells, NKT cells, mesenchymal stem cell, or pluripotent stem cells.

As used herein, the term “essentially free,” refers to a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, the terms “inhibiting,” “reducing,” or “prevention,” or any variation of these terms, when used in the claims and/or the specification includes any measurable decrease or complete inhibition to achieve a desired result.

As used herein, the term “effective” refers to an amount of the present disclosure that is adequate to accomplish a desired, expected, or intended result.

As used herein, the terms “immune system cell” or “immune cell” refer to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells, including Natural Killer T (NK-T) cells). Exemplary immune system cells include a CD4⁺ T cell, a CD8⁺ T cell, a CD4⁻CD8⁻ double negative T cell, a γδ T cell, a regulatory T cell, a natural killer cell, a natural killer T cell, and a dendritic cell. Macrophages and dendritic cells may be referred to as “antigen presenting cells” or “APCs,” which are specialized cells that can activate T cells when a major histocompatibility complex (MEW) receptor on the surface of the APC complexed with a peptide interacts with a TCR on the surface of a T cell.

As used herein, the term “Major Histocompatibility Complex” (MHC) refers to glycoproteins that deliver peptide antigens to a cell surface. MHC class I molecules are heterodimers having a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where a peptide:MHC complex is recognized by CD4⁺ T cells. Human MHC is referred to as human leukocyte antigen (HLA). HLA-II types include DP, DM, DOA, DOB, DQ, and DR. Numerous alleles encoding the subunits of the various HLA types are known, including, for example, HLA-DQA1*03, HLA-DQB1*0301, HLA-DQB1*0302, HLA-DQB1*0303.

As used herein, the terms “T cell” or “T lymphocyte” refers to an immune system cell that matures in the thymus and produces T cell receptors (TCRs). T cells can be naive (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of: central memory T cells (T_{CM}, increased expression of CD62L, CCR7, CD28, CD127, CD45RO, and CD95, and decreased expression of CD54RA as compared to naive T cells); and effector memory T cells (T_{EM}, decreased expression of CD62L, CCR7, CD28, CD45RA, and increased expression of CD127 as compared to naive T cells or TC_{M}).

As used herein, “effector T cells” (T_{E}) refers to antigen-experienced CD8+ cytotoxic T lymphocytes that have decreased expression of CD62L, CCR7, CD28, and are positive for granzyme and perforin as compared to TC_{M}. Helper T cells (T_{H}) are CD4+ cells that influence the activity of other immune cells by releasing cytokines. CD4⁻ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺CD25⁺ (Foxp3⁺) regulatory T cells and Treg17 cells, as well as Tr1, Th3, CD8⁺CD28⁻, and Qa-1 restricted T cells.

As used herein, the term “T cell receptor” (TCR) refers to an immunoglobulin superfamily member having a variable binding domain, a constant domain, a transmembrane region, and a short cytoplasmic tail (see, e.g., Janeway et al., Immunobiology: The Immune System in Health and Disease, 3.sup.rd Ed., Current Biology Publications, p. 4:33, 1997, relevant portions incorporated herein by reference) capable of specifically binding to an antigen peptide bound to, or presented by, a MHC. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains: a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed., relevant portions incorporated herein by reference) at the N-terminus; and one constant domain (e.g., α-chain constant domain or Cα, typically amino acids 117 to 259 based on Kabat, β-chain constant domain or Cβ, typically amino acids 117 to 295 based on Kabat) adjacent to the cell membrane. Also, like immunoglobulins, the variable domains contain complementary determining regions (CDRs) separated by framework regions (FRs) (see, e.g., Jones et al., Proc. Nat'l Acad. Sci. U.S.A. 87:9138, 1990; Chothia et al., EMBO 1 7:3745, 1988; see also Lefranc et al., Dev. Comp. Immunol. 27:55, 2003, relevant portions incorporated herein by reference). TCR variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300, relevant portions incorporated herein by reference). A numbering scheme provides a standardized delineation of framework regions and CDRs in the TCR variable domains.

In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

As used herein, the term “CD3” refers to a multi-protein complex of six chains (see, Abbas and Lichtman, 2003; Janeway et al., p. 172 and 178, 1999, relevant portions incorporated herein by reference). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif (ITAM), whereas each CD3ζ chain has three ITAMs. ITAMs are important for the signaling capacity of a TCR complex. CD3 as used in the present disclosure may be from various animal species, including human, mouse, rat, or other mammals.

As used herein, the term “TCR complex” refers to a complex formed by the association of CD3 with TCR. For example, a TCR complex can be composed of a CD3γ chain, a CD3δ chain, two CD3ε chains, and a homodimer of CD3ζ chains, a TCRα chain, and a TCRβ chain. Alternatively, a TCR complex can be composed of a CD3γ chain, a CD3δ chain, two CD3ε chains, and a homodimer of CD3ζ chains, a TCRγ chain, and a TCRδ chain.

As used herein, the term “component of a TCR complex,” refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRα, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, the term “CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB P01730, relevant portions incorporated herein by reference). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHC-II α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732, relevant portions incorporated herein by reference).

As used herein, the terms “variable region” or “variable domain” refers to the domain of a TCR α-chain or β-chain (or γ-chain and δ-chain for γδ TCRs) that is involved in binding of the TCR to antigen. The variable domains of the α-chain and β-chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). In certain cases, a single Vα or Vβ domain may be sufficient to confer antigen-binding specificity. TCRs that bind a particular antigen may be isolated using a Vα or Vβ domain from a TCR that binds the antigen to screen a library of complementary Vα or Vβ domains, respectively.

As used herein, the terms “complementarity determining region,” and “CDR,” are synonymous with “hypervariable region”, and refer to non-contiguous sequences of amino acids within TCR variable regions that confer antigen specificity and/or binding affinity to the TCR. In general, there are three CDRs in each α-chain variable region αCDR1, αCDR2, αCDR3) and three CDRs in each β-chain variable region (βCDR1, βCDR2, βCDR3). CDR3 is thought to be the main CDR responsible for recognizing processed antigen. CDR1 and CDR2 mainly interact with the MHC.

As used herein, the terms “nucleic acid” or “nucleic acid molecule” refer to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

As used herein, the term “isolated” refers to a material that is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, the term “gene” refers to a segment of DNA involved in producing a polypeptide chain and can includes regions preceding and following the coding region as well as intervening sequences (introns) between individual coding segments (exons).

As used herein, the term “recombinant” refers to a cell, microorganism, nucleic acid molecule, or vector that has been genetically engineered by human intervention-13 that is, modified by introduction of a heterologous nucleic acid molecule, or refers to a cell or microorganism that has been altered such that expression of an endogenous nucleic acid molecule or gene is controlled, deregulated, deleted, attenuated, or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, the term “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

As used herein, the term a “conservative substitution” refers to a substitution of one amino acid for another amino acid that has similar properties. Exemplary conservative substitutions are well known in the art (see, e.g., WO 97/09433 at page 10; Lehninger, Biochemistry, 2.sup.nd Edition; Worth Publishers, Inc. NY, N.Y., pp. 71-77, 1975; Lewin, Genes IV, Oxford University Press, NY and Cell Press, Cambridge, Ma., p. 8, 1990, relevant portions incorporated herein by reference).

As used herein, the term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Generally, vectors are those capable of autonomous replication (episomal vector) or expression of nucleic acid molecules to which they are linked (expression vectors). Viral vectors can include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996, relevant portions incorporated herein by reference).

As used herein, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

As used herein, the term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

As used herein, the term “introduced” in the context of inserting a nucleic acid molecule into a cell, is also known to be achieved by “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, a “heterologous” nucleic acid molecule, construct or sequence refers to a nucleic acid molecule or portion of a nucleic acid molecule that is not native to a host cell but may be homologous to a nucleic acid molecule or portion of a nucleic acid molecule from the host cell. The source of the heterologous nucleic acid molecule, construct or sequence may be from a different genus or species. In certain embodiments, a heterologous nucleic acid molecule is added (i.e., is not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by a heterologous polynucleotide introduced into the host cell, even if the host cell encodes a homologous protein or activity.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous nucleic acid molecules encoding desired binding proteins specific for an apolipoprotein B (ApoB) peptide (e.g., TCRα and TCRβ). When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules, or protein activities, refer to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the terms “homologous” or “homolog” refer to a molecule or activity found in or derived from a host cell, species or strain. For example, a heterologous polynucleotide may be homologous to a native host cell gene, and may optionally have an altered expression level, a different sequence, an altered activity, or any combination thereof.

As used herein, the term “sequence identity,” refers to the percentage of amino acid residues (or a polynucleotide) in one sequence that are identical with the amino acid residues (or a polynucleotide) in another reference polypeptide sequence (or a polynucleotide sequence) after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values, relevant portions incorporated herein by reference.

As used herein, a “hematopoietic progenitor cell” is a cell that can be derived from hematopoietic stem cells (such as bone marrow or fetal tissue) that is capable of further differentiation into mature cells types (e.g., immune system cells). Exemplary hematopoietic progenitor cells include those with a CD24^{lo}Lin⁻CD117⁺ phenotype or those found in the thymus (referred to as progenitor thymocytes).

As used herein, the term “host” refers to a cell (e.g., a T cell) such as a mammalian insect, plant, yeast, or microorganism targeted for genetic modification with a heterologous nucleic acid molecule to produce the T cell Receptor alpha and/or beta chain polypeptide(s) of interest, specifically, apolipoprotein B (ApoB) specific TCR when seen in the context of Class II MHC. In certain embodiments, a host cell may optionally possess or be modified to include other genetic modifications that confer desired properties related or unrelated to biosynthesis of the T cell Receptor alpha and/or beta chain polypeptide(s) of interest, specifically, apolipoprotein B (ApoB) specific TCR. In certain embodiments, a host cell is a human hematopoietic progenitor cell transduced with a heterologous nucleic acid molecule encoding a TCRα/TCRβ chain specific for apolipoprotein B (ApoB) peptide.

T Cell Receptor (TCR). The genetically engineered antigen receptors include recombinant T cell receptors (TCRs) and/or TCRs cloned from naturally occurring T cells.

As used herein, the term “T cell receptor” or “TCR” refers to a molecule that includes a variable α and β chains (also known as TCRα and TCRβ, respectively) or a variable γ and δ chains (also known as TCRγ and TCRδ, respectively) and that is capable of specifically binding to a apolipoprotein B (ApoB) peptide bound to a MHC receptor. In some embodiments, the TCR is in the αβ form. In certain embodiments, the engineered TCR has an alpha chain of SEQ ID NO:376 and a beta chain of SEQ ID NO: 364 with a CDR3 having the amino acid sequence of SEQ ID NO: 179 to 356, see Table 12. In some embodiments, the TCR comprises an alpha chain having at least 90, 95, 96, 97, 98, or 99% identity to a nucleotide sequence that encodes SEQ ID NO: 376 and a beta chain having at least 95, 96, 97, 98, or 99% identity to a nucleotide sequence that encodes SEQ ID NO: 264, with a CDR3 selected from SEQ ID NOS: 1-178, see Table 12.

In certain embodiments, the engineered TCR has an alpha chain and a beta chain with a CDR3 having the amino acid sequence selected from those set forth in PMID: 35766025, Circ Res. 2022 Jun. 29; 101161CIRCRESAHA122321116. Doi: 10.1161/CIRCRESAHA.122.321116, entitled “Immunodominant MHC-II Restricted Epitopes in Human Apolipoprotein B, Roy et al., herein incorporated by reference in its entirety. In some embodiments, the TCR comprises an alpha chain having at least 90, 95, 96, 97, 98, or 99% identity to a nucleotide sequence that encodes SEQ ID NO: 376 and a beta chain having at least 95, 96, 97, 98, or 99% identity to a nucleotide sequence that encodes an amino acid sequence set forth in PMID: 35766025, Circ Res. 2022 Jun. 29; 101161CIRCRESAHA122321116. Doi: 10.1161/CIRCRESAHA.122.321116, entitled “Immunodominant MHC-II Restricted Epitopes in Human Apolipoprotein B, Roy et al., herein incorporated by reference in its entirety.

Generally, TCRs exist in αβ and γδ forms, but T cells expressing them may have distinct locations in the body and/or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, Immunobiology: The Immune System in Health and Disease, 3.sup.rd Ed., Current Biology Publications, p. 433, 1997), relevant portions incorporated herein by reference. For example, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. As used herein, the term “TCR” should be understood to also include functional TCR fragments thereof. The term also encompasses intact or full-length TCRs, including TCRs in the αβ form or γδ form when having the CDR1,2, and/or 3 disclosed herein.

As used herein, an “antigen-binding portion” or “antigen-binding fragment” of a TCR, which are used interchangeably, refers to a molecule that contains a portion of the structural domains of a TCR and that binds the antigen in the context of an MHC protein, referred to as an MHC-peptide complex, to which the full TCR binds. An antigen-binding portion contains the variable domains of a TCR, such as variable a chain and variable β chain of a TCR, sufficient to form a binding site for binding to a specific MHC-peptide complex, such as generally where each chain contains three complementarity determining regions.

It is known that the variable domains of the TCR chains associate to form complementarity determining regions (CDRs) analogous to immunoglobulins, which confer antigen recognition and determine peptide specificity by forming the binding site of the TCR molecule, determine peptide specificity, and determine the MHC molecules that forms the peptide-MHC complex. Like immunoglobulins, TCR CDRs are separated by framework regions (FRs) (see, e.g., Jores et al., PNAS U.S.A. 87:9138, 1990; Chothia et al., EMBO J. 7:3745, 1988; see also Lefranc et al., Dev. Comp. Immunol. 27:55, 2003), relevant portions incorporated herein by reference. In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane, relevant portions incorporated herein by reference. Generally, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond linking the two chains. In certain examples, the TCR may have an additional cysteine residue in each of the α and β chains such that the TCR contains two disulfide bonds in the constant domains.

Generally, TCR chains contain a transmembrane domain, although that can be removed, or replaced with other transmembrane domain(s). Often, the transmembrane domain is positively charged. Generally, TCR contains a cytoplasmic tail, although that can be removed, or replaced with other cytoplasmic tail(s). Generally, the structure allows the TCR to associate with other molecules of the CD3 complex. For example, the TCR containing constant domains with a transmembrane region can anchor the protein in the cell membrane and associate with invariant subunits of the CD3 signaling complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell.

Generally, the TCR may be a heterodimer of two chains α and β (or γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., an APOB antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g., cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T-cells can be a cultured T cell hybridoma or clone. In some embodiments, the TCR clone for a target antigen has been generated in transgenic mice engineered with human immune system genes (e.g., the human leukocyte antigen system, or HLA). In some embodiments, phage display is used to isolate TCRs against a target antigen (see, e.g., Varela-Rohena et al. (2008) Nat Med. 14: 1390-1395 and Li (2005) Nat Biotechnol. 23:349-354, relevant portions incorporated herein by reference). In some examples, the TCR or antigen-binding portion thereof is synthetically generated from knowledge of the sequence of the TCR.

Chimeric T Cell Receptors. The present disclosure also includes engineered antigen receptors include chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December, 2013), relevant portions incorporated herein by reference. The CARs generally include an extracellular antigen (or ligand) binding domain linked to one or more intracellular signaling components, and in some aspects, via linkers and/or transmembrane domain(s). Generally, these molecules mimic or approximate a signal through a natural antigen receptor, a signal through such a receptor in combination with a costimulatory receptor, and/or a signal through a costimulatory receptor alone. The T cell CAR can be used to replace an antigen-binding portion or portions of an antibody molecule, to make a single-chain antibody fragment (scFv) derived from the variable heavy (VH) and variable light (VL) chains of a monoclonal antibody (mAb).

The arrangement of the antigen-binding domain of a CAR may be multimeric, such as dimeric or multimeric. The multimers can be formed by cross pairing of the variable portions of the light and heavy chains. A hinge portion of the CAR may be shortened or deleted to make a CAR with a single antigen binding domain, a transmembrane region and an intracellular signaling domain. Also, the Fc portion of an antibody may be deleted from scFv used to as an antigen-binding region to generate CARs according to the present disclosure. In some embodiments, an antigen-binding region may encode just one of the Fc domains, e.g., either the CH2 or CH3 domain from human immunoglobulin. One may also include the hinge, CH2, and CH3 region of a human immunoglobulin that has been modified to improve dimerization and oligomerization. In some embodiments, the hinge portion of may comprise or consist of a 8-14 amino acid peptide (e.g., a 12 AA peptide), a portion of CD8α, or the IgG4 Fc. In some embodiments, the antigen binding domain may be suspended from cell surface using a domain that promotes oligomerization, such as CD8α.

The intracellular signaling domain of a CAR will generally cause the activation of at least one of the normal effector functions of an immune cell comprising the CAR. For example, the intracellular domain may promote an effector function of a T cell such as, e.g., cytolytic activity or helper activity including the secretion of cytokines. The effector function in a naive, memory, or memory-type T cell may include antigen-dependent proliferation. As used herein, the term “intracellular signaling domain” refers to the portion of a CAR that can transduce the effector function signal and/or direct the cell to perform a specialized function. While usually the entire intracellular signaling domain may be included in a CAR, in some cases a truncated portion of a cytoplasmic domain may be included.

For example, the TCR intracellular domains may be engineered to have the zeta chain of the T cell receptor or any of its homologs (e.g., zeta, delta, gamma, or epsilon), MB1 chain, B29, Fc RIII, Fc RI, and combinations of signaling molecules, such as CD3C and CD28, CD27, 4-1BB, DAP-10, OX40, and combinations thereof, as well as other similar molecules and fragments. Intracellular signaling portions of other members of the families of activating proteins can be used, such as FcγRIII and FcεRI. Examples of these alternative transmembrane and intracellular domains can be found, e.g., Gross et al. (1992), Stancovski et al. (1993), Moritz et al. (1994), Hwu et al. (1995), Weijtens et al. (1996), and Hekele et al. (1996), which are incorporated herein by reference in their entirety. The transmembrane and/or intracellular domain may include a sequence encoding a costimulatory receptor such as, e.g., a modified CD28 intracellular signaling domain, CD28, CD27, OX-40 (CD134), DAP10, or 4-1BB (CD137) costimulatory receptor. In some embodiments, both a primary signal initiated by CD3ζ, an additional signal provided by a human costimulatory receptor may be included in a CAR to more effectively activate transformed T cells, which may help improve in vivo persistence and the therapeutic success of the adoptive immunotherapy. The CAR may be engineered with transmembrane domain(s), e.g., the human IgG4 Fc hinge and Fc regions, the human CD4 transmembrane domain, the human CD28 transmembrane domain, the transmembrane human CD3ζ domain, or a cysteine mutated human CD3ζ domain, or a transmembrane domains from a human transmembrane signaling protein such as, e.g., the CD16 and CD8 and erythropoietin receptor.

An isolated nucleic acid segment and expression cassette including DNA sequences that encode a CAR may be generated that include a human TCR alpha chain which is: HGNC: 12027 NCBI Entrez Gene: 6955 UniProtKB/Swiss-Prot: PODSE1 UniProtKB/Swiss-Prot: PODTU3, incorporated herein by reference having at least 90, 95, 96, 97, 98, or 99% identity to the nucleotide sequence of NCBI Entrez Gene: 6955 and/or a human TCR beta chain having at least 90, 95, 96, 97, 98, or 99% identity to HGNC: 12155 NCBI Entrez Gene: 6957 UniProtKB/Swiss-Prot: P0DSE2 UniProtKB/Swiss-Prot: P0DTU4.

```
Human TCR Alpha Chain (SEQ ID NO: 376):
                10 20 30 40 50
MVLKESVSIL WIQLAWVSTQ LLEQSPQFLS IQEGENLTVY CNSSSVESSL
                60 70 80 90 100
QWYRQEPGEG PVLLVTVVTG GEVKKLKRLT FQFGDARKDS SLHITAAQPG
              110 120 130 140 150
DTGLYLCAGG GSQGNLIFGK GTKLSVKPIQ NPDPAVYQLR DSKSSDKSVC
              160 170 180 190 200
LFTDFDSQTN VSQSKDSDVY ITDKTVLDMR SMDEKSNSAV AWSNKSDFAC
              210 220 230 240 250
ANAFNNSIIP EDTFFPSPES SCDVKLVEKS FETDINLNFQ NLSVIGERIL
              260
LLKVAGENLL MTLRLWSS
Human TCR Beta Chain (SEQ ID NO: 377):
                10 20 30 40 50
MSNQVLCCVV LCLLGANTVD GGITQSPKYL FRKEGQNVTL SCEQNLNHDA
                60 70 80 90 100
MYWYRQDPGQ GLRLIYYSQI VNDFQKGDIA EGYSVSREKK ESFPLTVTSA
              110 120 130 140 150
QKNPTAFYLC ASSIRSSYEQ YFGPGTRLTV TEDLKNVFPP KVAVFEPSEA
              160 170 180 190 200
EISHTQKATL VCLATGFYPD HVELSWWVNG KEVHSGVSTD PQPLKEQPAL
              210 220 230 240 250
NDSRYCLSSR LRVSATFWQN PRNHFRCQVQ FYGLSENDEW TQDRAKPVTQ
              260 270 280 290 300
IVSAEAWGRA DCGFTSESYQ QGVLSATILY EILLGKATLY AVLVSALVLM
              310
AMVKRKDSRG
```

A variety of vectors may be used for delivery of the DNA encoding a CAR to immune such as T cells. CAR expression may be under the control of regulated eukaryotic promoter such as, the CMV promoter, EF1alpha promoter, or Ubiquitin promoter. The vector may also contain a selectable marker to facilitate their manipulation in vitro. In some embodiments, the CAR can be expressed from mRNA in vitro transcribed from a DNA template. These constructs can be made following any number of protocols, such as those taught by Sambrook et al., Molecular Cloning: A Laboratory Manual, 3.sup.rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994, relevant portions incorporated herein by reference.

In some embodiments, the present disclosure provides soluble TCRs. Soluble TCRs are useful, not only for the purpose of investigating specific TCR-peptide-MHC interactions, but as a diagnostic tool to detect atherosclerosis-related autoimmune disease, or to detect atherosclerosis-related autoimmune disease markers. Soluble TCRs can be used for staining, e.g., to determine the presence of the apolipoprotein B (ApoB) peptide in the context of the MHC-Class II. Soluble TCRs can also be used to deliver a therapeutic agent, for example a cytotoxic compound, to cells presenting the apolipoprotein B (ApoB) peptide.

Adoptive Cell Transfer Therapy. The TCR of the present disclosure can also be used for adoptive cell transfer therapy of immune cells, such as autologous or allogeneic T cells, or even, regulatory T cells, CD4+ T cells, CD8+ T cells, gamma-delta T cells, NK cells, invariant NK cells, NKT cells, mesenchymal stem cell, or pluripotent stem cells) therapy are transfected to express the TCR or binding fragments thereof that bind the apolipoprotein B (ApoB) peptide. Generally, adoptive T cell therapies include genetically engineered TCR-transduced T cells by expressing an alpha chain having at least 90, 95, 96, 97, 98, or 99% identity to the amino acid sequence of TCR alpha and/or a beta chain having at least 90, 95, 96, 97, 98, or 99% identity to the amino acid sequence of TCR beta, or binding fragments of each (See Table 12). The immune cells thus engineered are provided for the treatment of atherosclerosis-related autoimmune disease comprising introducing into the subject the engineered cells, such as the engineered T cells. Often, the adoptive cell transfer therapy is provided to a human patient in combination with as second therapy, such as a chemotherapy, a radiotherapy, a surgery, or a second immunotherapy.

The TCR-engineered cells of the present disclosure are provided to a subject as an immunotherapy to target atherosclerosis-related autoimmune disease. T cells transfected to express the TCR of the present disclosure will often be autologous but can be allogeneic. For making the TCR engineered T cells, autologous T cells are isolated from the patient and are modified to express the TCR of the present disclosure. If the T cells are allogeneic, these are often pooled from several donors or can be T cell clones. The engineered T cells are administered to the subject of interest in an amount sufficient to control, reduce, or eliminate symptoms and signs of the disease being treated.

The isolated T cells can be obtained from blood, bone marrow, lymph, umbilical cord, or lymphoid organs. Generally, the T cells are primary cells, such as those isolated directly from a subject and/or isolated from a subject and frozen. The cells can include one or more subsets of T cells or other cell types, such as whole T cell populations, or isolated subpopulations of T cells, such as CD4+ cells, CD8+ cells, and subpopulations thereof, which can be further divided by function, activation state, maturity, potential for differentiation, expansion, recirculation, localization, presence in a particular organ or compartment, marker or cytokine secretion profile, and/or degree of differentiation.

Sub-types and subpopulations of T cells for use with the present disclosure can be, e.g., CD4+ and/or CD8+ T cells, naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, or follicular helper T cells.

T cells can be pooled and rapidly expanded to provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days. T cells can be rapidly expanded using non-specific T cell receptor stimulation in the presence of feeder lymphocytes and either interleukin-2 (IL-2) or interleukin-15 (IL-15), non-specific T cell receptor stimulus such as OKT3. T cells can be rapidly expanded by stimulation of peripheral blood mononuclear cells (PBMC) in vitro with one or more antigens in the presence of a T cell growth factor, such as IL-2 or IL-15.

The engineered immune cells of the present disclosure may be administered intravenously, intramuscularly, subcutaneously, transdermally, intraperitoneally, intrathecally, parenterally, intrathecally, intracavitary, intraventricularly, intra-arterially, or via the cerebrospinal fluid, or by any implantable or semi-implantable, permanent or degradable device. The appropriate dosage of the engineered immune cell therapy, such as engineered T cells, may be determined based on the type of disease to be treated, severity and course of the disease, the clinical condition of the individual, the individual's clinical history and response to the treatment, and knowledge and skill of an attending physician.

The engineered immune cells may be made into a pharmaceutical composition or made into an implant appropriate for administration in vivo, with appropriate carriers or diluents, that are pharmaceutically acceptable. The introduction of the cells of the present disclosure can follow the guidance described in the art (see, for instance, Remington's Pharmaceutical Sciences, 16th Ed., Mack, ed. (1980), relevant portions incorporated herein by reference). Generally, transduced T cells expressing a CAR can be formulated into a preparation in liquid or semisolid form. Generally, a pharmaceutically acceptable form is employed that does not kill or reduce the effectiveness of the cells expressing the chimeric receptor. Thus, the engineered T cells can be made into a pharmaceutical composition containing a balanced salt solution such as Hanks' balanced salt solution, or normal saline.

The present disclosure can also be delivered by any number of vectors, liposomes, or even naked DNA to introduce the TCR into host cells, such as host immune cells. Methods of stably transfecting T cells by electroporation using naked DNA are known in the art. Naked DNA generally refers to the DNA encoding a TCR of the present disclosure in a plasmid expression vector under the control of a promoter that drives expression. Alternatively, the present disclosure can be delivered using a viral vector (e.g., a retroviral vector, adenoviral vector, adeno-associated viral vector, or lentiviral vector) that introduces the chimeric construct into T cells. Generally, a vector encoding a CAR that is used for transfecting a T cell from a subject should generally be non-replicating in the subject's T cells. A large number of vectors are known that are based on viruses, where the copy number of the virus maintained in the cell is low enough to maintain viability of the cells, such as, pFB-neo vectors or vectors based on SV40, HIV, HSV, EBV, or BPV.

Nucleic Acids. The present disclosure includes polynucleotides encoding an isolated TCR, CAR, or soluble peptide the TCR comprises an alpha chain having at least 90, 95, 98, or 99% identity to a human TCR alpha chain and a human TCR beta chain CDR3 having at least 95, 96, 97, 98, or 99% identity to the a sequence of SEQ ID NOS: 1 to 178. The term “nucleic acid” is intended to include DNA and RNA and can be either double stranded or single stranded.

A recombinant expression vector contains one or more of the polynucleotides of the present disclosure, as well as, regulatory sequences selected on the basis of the host cells to be used for expression, to which the one or more polynucleotides are operatively linked. As used herein, the terms “operatively linked” or “operably linked” refer to the one or more polynucleotide(s) linked to regulatory sequences to allow expression of the one or more polynucleotide(s).

The present disclosure includes an engineered T cell receptor (TCR) comprising a human alpha chain and/or a beta chain CDR3 having the amino acid sequence of SEQ ID NOS: 179 to 356, wherein the TCR is specific for an apolipoprotein B (ApoB) peptide, See Table 12. In one aspect, the engineered TCR binds to the apolipoprotein B (ApoB) peptide in a complex with HLA DRB5*01:01. In another aspect, the TCR comprises an alpha chain and a beta chain having at least 90, 95, 98, or 99% sequence identity to the amino acid sequence of SEQ ID NO: 179-232, encoded by polynucleotide having at least 90, 95, 96, 97, 98, or 99% sequence identity to SEQ ID NOS: 1-54, respectively. In another aspect, the TCR is humanized. In another aspect, the TCR comprises an alpha chain and a beta chain having at least 90, 95, 96, 97, 98, or 99% identity to the nucleotide sequence of SEQ ID NOS:1-134. In another aspect, the TCR is further defined as a soluble TCR, wherein the soluble TCR does not comprise a transmembrane domain, or comprises transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the TCR further comprising a detectable label. In another aspect, the TCR is covalently bound to a therapeutic agent, an immunotoxin or a chemotherapeutic agent. In another aspect, and the CDR3 is selected from SEQ ID NO: 179 to 356 and an alpha chain. In another aspect, the TCR is part of a multivalent TCR complex comprising a plurality of TCRs. In another aspect, the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule.

The present disclosure includes a polypeptide encoding the TCR comprising an alpha chain and a beta chain CDR3 having the amino acid sequence of SEQ ID NO: 179 to 356, wherein the TCR is specific for apolipoprotein B (ApoB) peptide (See Table 12).

The present disclosure includes a polynucleotide encoding TCR polypeptide(s) comprising an alpha chain and beta chain CDR3 having the amino acid sequence of SEQ ID NO: 179 to 356, wherein the TCR is specific for an apolipoprotein B (ApoB) peptide (See Table 12).

The present disclosure includes an expression vector encoding TCR polypeptide(s) comprising an alpha chain and a beta chain CDR3 having the amino acid sequence of SEQ ID NO: 179 to 356, wherein the TCR is specific for an apolipoprotein B (ApoB) peptide. In one aspect, the sequence encoding the TCR is under the control of a promoter. In another aspect, the expression vector is a viral or a retroviral vector. In another aspect, the vector further encodes a linker domain positioned between the alpha chain and beta chain. In another aspect, the linker domain comprises one or more protease cleavage sites, or wherein the one or more cleavage sites are separated by a spacer.

The present disclosure includes a host cell engineered to express a polypeptide encoding the TCR comprising an alpha chain and beta chain CDR3 having the amino acid sequence of SEQ ID NO: 179 to 356, wherein the TCR is specific for an apolipoprotein B (ApoB) peptide. In another aspect, the cell is a T cell, NK cell, invariant NK cell, NKT cell, mesenchymal stem cell (MSC), or induced pluripotent stem (iPS) cell. In another aspect, the host cell is an immune cell. In another aspect, the T cell is a CD8⁺ T cell, CD4⁺ T cell, or γδ T cell. In another aspect, the T cell is a regulatory T cell (Treg). In another aspect, the host cell is autologous or allogeneic.

The present disclosure includes a method for engineering a host cell comprising contacting an immune cell with the TCR or the expression vector of the present disclosure. In one aspect, the method comprises contacting is further defined as transfecting or transducing, wherein transfecting comprises electroporating RNA encoding the TCR described hereinabove into the immune cell.

The present disclosure includes a method for treating a subject with an atherosclerosis-related autoimmune disease comprising an apolipoprotein B (ApoB) peptide, the method comprising: administering to the subject an effective amount of one or more immune cells modified by cloning genes of the alpha and beta chains of a T cell receptor (TCR) ex vivo to express a chimeric antigen receptor specific for the apolipoprotein B (ApoB), wherein the chimeric antigen receptor comprises an alpha chain and a beta chain CDR3 having the amino acid sequence of SEQ ID NO: 179 to 356. In one aspect, the immune cell is T cell, NK cell, invariant NK cell, NKT cell, mesenchymal stem cell (MSC), or induced pluripotent stem (iPS) cell, or a peripheral blood lymphocyte. In another aspect, method further comprises at least one of: sorting the immune cells into T cells to isolate TCR engineered T cells; performing a T cell cloning of the immune cells by serial dilution; or expanding a T cell clone from the immune cells by a rapid expansion protocol. In another aspect, the subject is identified to have an HLA DRB5*01:01 allele. In another aspect, the immune cell is a T cell selected from a CD8⁺ T cell, CD4⁺ T cell, or Treg. In another aspect, one or more immune cells are administered intravenously, intraperitoneally, intratracheally, intramuscularly, endoscopically, intralesionally, percutaneously, subcutaneously, regionally, or by direct injection or perfusion.

The present disclosure includes chimeric antigen receptor expressing T cell (CAR-T) comprising an antigen recognition moiety and a T-cell activation moiety, wherein the T-cell activation moiety comprises a transmembrane domain, and wherein the antigen recognition moiety is directed against apolipoprotein B (ApoB). In another aspect, the transmembrane domain is a CD28 transmembrane domain or a CD8a transmembrane domain. In another aspect, the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the antigen recognition moiety comprises the amino acid sequence of wherein the TCR comprises an alpha chain variable region and a beta chain variable region with a CDR3 having at least 90, 95, 96, 97, 98, or 99% identity to the amino acid sequence of SEQ ID NO: 179 to 356. In another aspect, the antigen recognition moiety comprises an alpha chain and a beta chain CDR3 having at least 90, 95, 96, 97, 98, or 99% identity to the amino acid sequence of SEQ ID NO: 179 to 356.

In certain embodiments, a binding protein of the present disclosure comprises an beta chain CDR3 having the amino acid sequence of SEQ ID NO: 179 to 356 and an alpha chain. The CDR1, CDR2, and CDR3 of the alpha and the CDR1, CDR2 of beta chains can be from a full-length human TCR sequences, respectively, wherein the beta chain CDR has at least 90, 95, 96, 97, 98, or 99% identity to the amino acid sequence of SEQ ID NO: 179 to 356, and in certain embodiments SEQ ID NOS: 179-232.

Broadly binding MHC Class-II restricted APOB peptides elicit antigen-specific responses in human CD4⁺ T cells.

The core HLA-II binding region of an epitope is a nonameric sequence. But because HLA-II molecules have an open-binding pocket, a 15-mer sequence is typically analyzed (3 aa overhang on each side). The entire 4563 aa long APOB protein contains 4549 possible 15-mer sequences. Those overlapping by >10 residues were removed because these sequences redundantly span the same nonameric core. Prediction tools available from Immune Epitope Database (IEDB) were used to score the remaining 911 peptides for their ability to bind a reference set of 27 most frequent and representative HLA DP, DQ and DR alleles (Table 11) which provide an estimated global coverage of >98% of individuals belonging to all the major races and ethnic groups³⁰.

TABLE 1
Average phenotypic frequencies of the
HLA-II alleles in IEDB's reference set

| | | Phenotype |
|---|---|---|
| Locus | Allele | frequency |
| DPB1 | DPB1*01:01 | 16.0 |
| | DPB1*02:01 | 17.5 |
| | DPB1*04:01 | 36.2 |
| | DPB1*04:02 | 41.6 |
| | DPB1*05:01 | 21.7 |
| | DPB1*14:01 | 7.4 |
| | Total | 94.5 |
| DQB1 | DQB1*02:01 | 11.3 |
| | DQB1*03:01 | 35.1 |
| | DQB1*03:02 | 19 |
| | DQB1*04:02 | 12.8 |
| | DQB1*05:01 | 14.6 |
| | DQB1*06:02 | 14.6 |
| | Total | 81.6 |
| DRB1 | DRB1*01:01 | 5.4 |
| | DRB1*03:01 | 13.7 |
| | DRB1*04:01 | 4.6 |
| | DRB1*04:05 | 6.2 |
| | DRB1*07:01 | 13.5 |
| | DRB1*08:02 | 4.9 |
| | DRB1*09:01 | 6.2 |
| | DRB1*11:01 | 11.8 |
| | DRB1*12:01 | 3.9 |
| | DRB1*13:02 | 7.7 |
| | DRB1*15:01 | 12.2 |
| | Total | 71.1 |
| DRB3/4/5 | DRB3*01:01 | 26.1 |
| | DRB3*02:02 | 34.3 |
| | DRB4*01:01 | 41.8 |
| | DRB5*01:01 | 16.0 |
| | Total | 87.7 |

Table adapted from Greenbaum et.al., Immunogenetic, 2011.

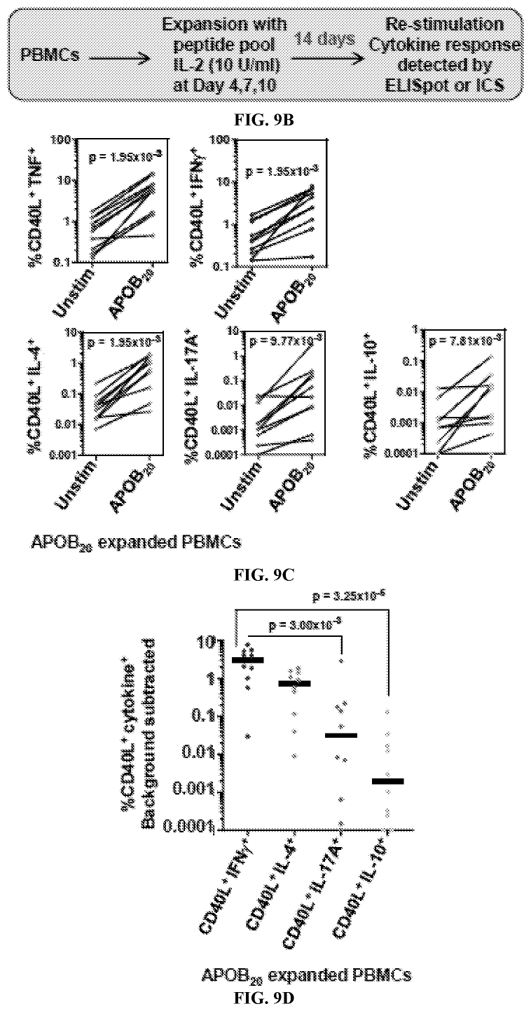

Peptides predicted to bind most (≥75%) of the alleles, were experimentally verified in HLA-II binding assays³¹. For each peptide, the number of alleles that bound at a <1000 nM threshold level was tabulated. Top 20 promiscuous binders (bind >50% of reference alleles) were selected for further study (FIG. 9A, Table 2).

TABLE 2
Details of the twenty APOB peptides
used in the study

| | | | No. of | |
|---|---|---|---|---|
| | | | HLA-II | SEQ |
| Peptide | | Start | alleles | ID |
| Number | Sequence | site | bound | NO: |
| 1 | DKRLAAYLMLMRSPS | 556 | 23 | 357 |
| 2 | TLTAFGFASADLIEI | 676 | 21 | 358 |
| 3 | FLHYIFMENAFELPT | 826 | 24 | 359 |
| 4 | VEFVTNMGIIIPDFA | 881 | 18 | 360 |
| 5 | VGSKLIVAMSSWLQK | 1226 | 21 | 361 |
| 6 | IKHIYAISSAALSAS | 1836 | 20 | 362 |
| 7 | HFSNVFRSVMAPFTM | 1891 | 22 | 363 |
| 8 | QLYSKFLLKAEPLAF | 1926 | 27 | 364 |
| 9 | LSQLQTYMIQFDQYI | 2171 | 17 | 365 |
| 10 | HVKHFVINLIGDFEV | 2316 | 21 | 366 |
| 11 | LIINWLQEALSSASL | 2491 | 17 | 367 |
| 12 | LEVLNFDFQANAQLS | 2801 | 18 | 368 |
| 13 | SLFFSAQPFEITAST | 3036 | 23 | 369 |
| 14 | GKIDFLNNYALFLSP | 3066 | 23 | 370 |
| 15 | RGLKLATALSLSNKF | 3391 | 23 | 371 |
| 16 | YKKLRTSSFALNLPT | 3771 | 21 | 376 |
| 17 | ILFSYFQDLVITLPF | 4241 | 17 | 372 |
| 18 | KFTYLINYIQDEINT | 4321 | 19 | 373 |
| 19 | QIHQYIMALREEYFD | 4376 | 25 | 374 |
| 20 | KIVSLIKNLLVALKD | 4406 | 20 | 375 |

This approach is supported by previous observations that the promiscuous HLA-II binders tend to be the most immunogenic^{27,32}. Homology analysis with BLAST and PEPMatch showed that these peptides did not share any significant sequence identity with other known epitopes in IEDB database³³. Using this APOB₂₀ peptide pool, a set of re-stimulation-based assays were optimized (Table 3).

TABLE 3
Re-stimulation protocols used to detect APOB-specific CD4 + T responses

| | Restimulation | | | |
|---|---|---|---|---|
| Assay | regime | PBMCs per well | Advantage | Disadvantage |
| Activation | 24 h stimulation | 1,000,000-2,000,000 | Sensitive detection | Not suitable for |
| Induced Marker | (ex vivo or | | of all antigen-specific | high throughput |
| (AIM) assay | after expansion) | | T cells irrespective | epitope screening |
| (Flow | in the presence | | of cytokine-secreting | |
| cytometry | of anti-CD40 | | potential, allows | |
| Based) | antibody to prevent | | isolation of | |
| | internalization | | Ag-specific live | |
| | of CD40L | | cells for sequencing | |
| ELISpot | 14 days expansion | 100,000-200,000 | Ideal for high | Limited |
| | 20-24 h | | throughput screening | information |
| | restimulation | | of peptides | (1-2 cytokines) |
| Intra cellular | 14 days expansion | 1,000,000 | Sensitive and | Viable cells of |
| staining (ICS) | 6 h restimulation, | | multiparametric. | interest not |
| (Flow | last 4 h with | | | retrievable, not |
| cytometry | Protein transport | | | suitable for |
| Based) | inhibitor | | | high-throughput |
| | | | | epitope |
| | | | | screening |

First, an in vitro expansion-based protocol was established (FIG. 9B) that maximizes the sensitivity and reliability of the quantitation of autoantigen-specific CD4⁺ T responses. Similar protocols have been used to resolve epitope-specific responses to self-antigens in other autoimmune-related diseases⁴. Expansion and restimulation of PBMCs with the APOB₂₀ pool and multiparametric flow cytometry-based intracellular cytokine staining (ICS) assay (antibodies in Table 4) detected significantly higher T helper cytokine responses in CD40L⁺CD4⁺ T activated cells, as compared to unstimulated controls (FIG. 9C). Most APOB-specific CD4⁺ T cells produced Th1 cytokines, some produced IL-4 and IL-17A. APOB₂₀-induced IL-10 was detectable but rare (median %₀CD40L⁺ IL-10⁺CD4⁺ T=0.002) (FIG. 9C, FIG. 9D)).

TABLE 4
Details of antibodies used in flow cytometry

| Group | Antibody | Fluorochrome | Clone | Company | Catalog |
|---|---|---|---|---|---|
| Dump | Anti-CD8 | APC-Cy7 | RPA-T8 | Biolegend | #301015 |
| channel | Anti-CD14 | APC-Cy7 | M5E2 | Biolegend | #301819 |
| | Anti-CD16 | APC-Cy7 | 3G8 | Biolegend | #302017 |
| | Anti-CD19 | APC-Cy7 | HIB19 | Biolegend | #302217 |
| | Anti-CD41 | APC-Cy7 | HIP8 | Biolegend | #303715 |
| | Anti-CD56 | APC-Cy7 | HCD56 | Biolegend | #318331 |
| T cell markers | Anti-CD3 | PerCp-Cy5.5 | UCHT1 | Biolegend | #300429 |
| | Anti-CD4 | Pacific Blue | RPA-T4 | Biolegend | #300524 |
| | Anti-CD8 | AF700 | RPA-T8 | Biolegend | #301027 |
| Activation | Anti-CD40L | PE | 24-31 | Thermo | #12-1548-41 |
| markers | | | | Fisher | |
| | Anti-CD69 | BV650 | FN50 | Biolegend | #310933 |
| | Anti-CD25 | PE-Cy7 | BC96 | Biolegend | #302611 |
| | Anti-OX-40 | APC | Ber- | Biolegend | #350007 |
| | | | ACT35 | | |
| | Anti-4-1BB | BV605 | 4B4-1 | Biolegend | #309821 |
| Memory | Anti-CCR7 | FITC | REA108 | Miltenyi | #130-117-812 |
| markers | Anti-CD45RA | AF700 | HI100 | Biolegend | #304119 |
| Cytokines | Anti-TNF | BV650 | Mab11 | Biolegend | #502937 |
| (in ICS) | Anti-IFNg | PE-Cy7 | 4S.B3 | Biolegend | #502527 |
| | Anti-IL-4 | PE-Dazzle594 | MP4-25D2 | Biolegend | #500831 |
| | Anti-IL-17A | APC | eBio64DEC17 | eBioscience | #17-7179-41 |
| | Anti-IL-10 | AF488 | JES3-9D7 | Biolegend | #501413 |
| Activation | Anti-CD40L | PE | TRAP1 | BD | |
| (in ICS) | | | | | |
| B cell marker | Anti-CD19 | APC-Cy7 | HIB19 | Biolegend | #302217 |
| HLA-II | Anti-HLA-DR | BV785 | L243 | Biolegend | #307641 |
| | Anti-HLA-DP | PE | B7/21 | BD | #566825 |
| Live/Dead | Fixable Ghost | BV510 | N/A | Tonbo | #13-0870-T100 |
| | viability dye | | | Biosciences | |

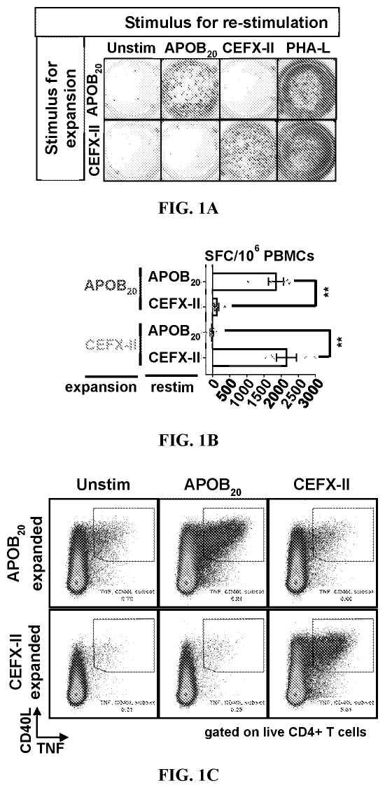

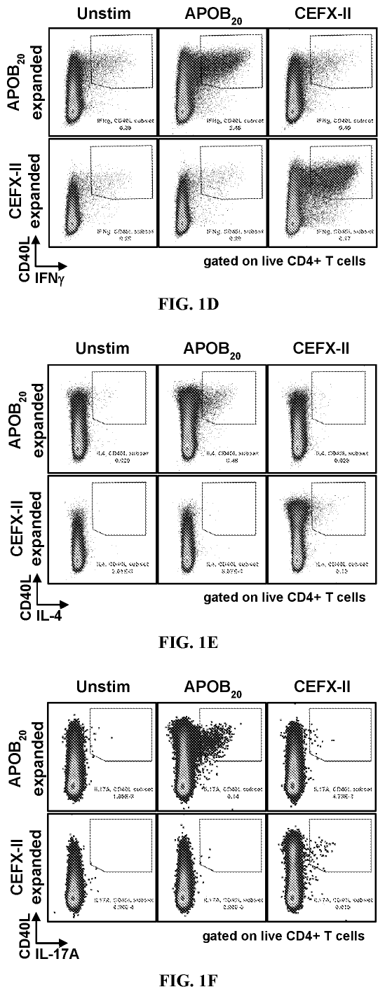

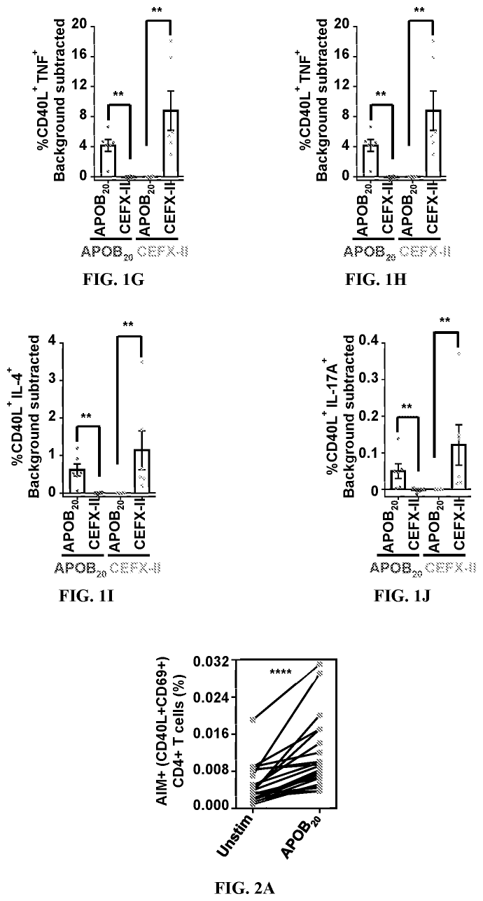

The efficacy and specificity of the expansion-based method was assessed in a crossover re-stimulation regime in which PBMCs were expanded with a peptide pool and then re-stimulated with either the cognate or an irrelevant pool. Next, they utilized two sets of peptide pools—the experimental APOB₂₀ pool and a positive control CEFX-II pool containing 68 epitopes from infectious agents, which are known to bind a broad range of HLA class-II alleles. PBMCs were expanded with either APOB₂₀ or CEFX-II pool. Subsequent re-stimulation on day 14 with the cognate pool elicited positive responses in an IFNγ ELISpot assay (FIG. 1A). Cross-over re-stimulation with the irrelevant pool yielded no responses. Unstimulated and PHA-L treated cells served as negative and positive controls, respectively. Average response magnitudes, expressed as spot forming cells (SFCs) per 10⁶ PBMCs, were significantly higher for the cognate pool than to the irrelevant pool (FIG. 1B). Specificity was further confirmed in an ICS assay for multiple CD4⁺ T helper cytokines: TNF (FIG. 1C), IFNγ (FIG. 1D), IL-4 (FIG. 1E) and IL-17A (FIG. 1F). Significant induction of cytokine response over unstimulated background controls was detected with the cognate, but not the irrelevant pool (FIGS. 1G-1J). This shows that the expansion process boosts sensitive detection of an APOB-specific T cell response and is strictly antigen specific.

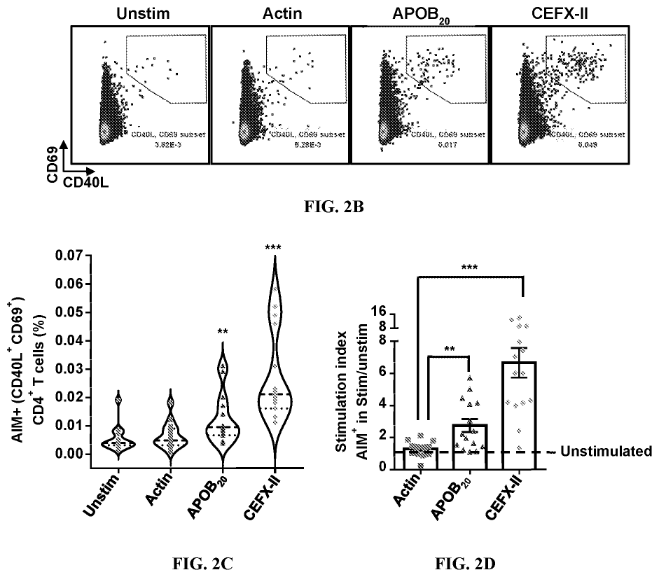

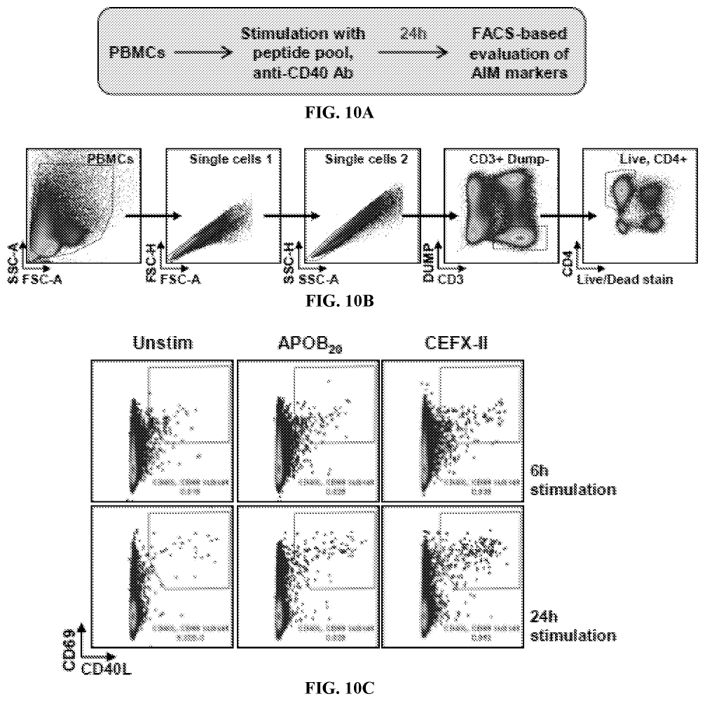

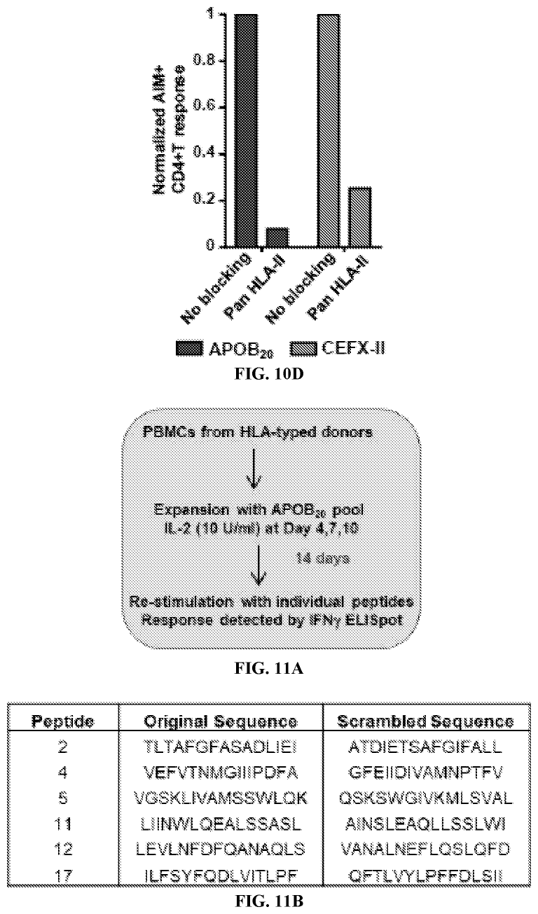

Significant autoreactive response to human APOB is detectable in the general population. Next, a 24 h assay was optimized (FIGS. 10A-10D) which is based on the surface expression of T cell activation-induced markers (AIM) and has been used in a range of studies to identify CD4⁺ T cell responses against vaccine candidates and pathogen-derived epitopes³⁴⁻³⁷. It has been previously established that the addition of anti-CD40 antibody during the stimulation phase inhibits CD40-CD40L interaction-induced internalization of CD40L and improves CD40L detection on the cell surface³⁸. It was found that the 24 h activation regime, as opposed to 6 h stimulation, improved the intensities of CD40L and CD69 surface expressions and lowered the background in unstimulated controls (FIG. 10C). This enhanced the signal-to-noise ratio of the antigen-specific CD4⁺ T response (FIG. 10C), allowing sensitive detection and accurate enumeration of low-frequency autoantigen-specific CD4⁺ T cells in ex vivo assays. HLA-II restriction was verified using a pan HLA-II blocking antibody which strongly inhibited APOB₂₀-induced AIM marker upregulation (FIG. 10D). Ex vivo stimulation of PBMCs from 21 donors (demographics in Table 5) with the APOB₂₀ pool for 24 h in the presence of anti-CD40 antibody significantly enhanced the frequencies of AIM⁺ (% CD40L⁺CD69⁺) CD4⁺ T cells in 100% of the donors tested (p<0.0001 versus unstimulated control, FIG. 2A).

TABLE 5
Summary of demographic characteristics of healthy subjects in screening cohort

| | | | | | Peptide- | |
|---|---|---|---|---|---|---|
| | AIM | | ELISpot | | specific | TCRb |
| | assay | | assay | | ICS assay | Immunoseq |
| Age | 22-46 | | 22-43 | | 23-43 | 24-37 |
| (years) | (median = 27, | | (median = 27, | | (median = 26, | (median = 25, |
| | IQR = 12.5) | | IQR = 12) | | IQR = 8) | IQR = 2) |
| Male | 38.1% | (8/21) | 36.8% | (7/19) | 20% (2/10) | 33.33% (2/6) |
| Female | 61.9% | (13/21) | 63.2% | (12/19) | 80% (8/10) | 66.67% (4/6) |

Next, APOB-specific CD4⁺ T responses were compared with those triggered by the positive control CEFX-II pool and a negative control pool containing 92 peptides spanning human α-actin protein (FIGS. 2B-2D). Actin is ubiquitously expressed and actin-specific T cells are known to undergo strong negative selection⁸. As compared to unstimulated controls (Mean % AIM⁺CD4⁺ T=0.005), stimulation with APOB₂₀ (Mean=0.012) and CEFX-II (Mean=0.03) pools, but not with the actin pool (Mean=0.006), resulted in significant increase in activated CD4⁺ T cells (FIGS. 2B, 2C). Stimulation-induced fold increase, commonly expressed as stimulation index (SI=AIM⁺CD4⁺ T cells in stimulated/unstimulated sets), was significantly higher for both APOB₂₀ (Mean SI=2.73, p=0.0027 versus actin, p=0.005 versus unstimulated) and CEFX-II (Mean SI=6.65, p=0.0001 versus actin or unstimulated) stimulations (FIG. 2D). There was no statistically significant autoimmune response to actin (Mean SI=1.28, n.s.).

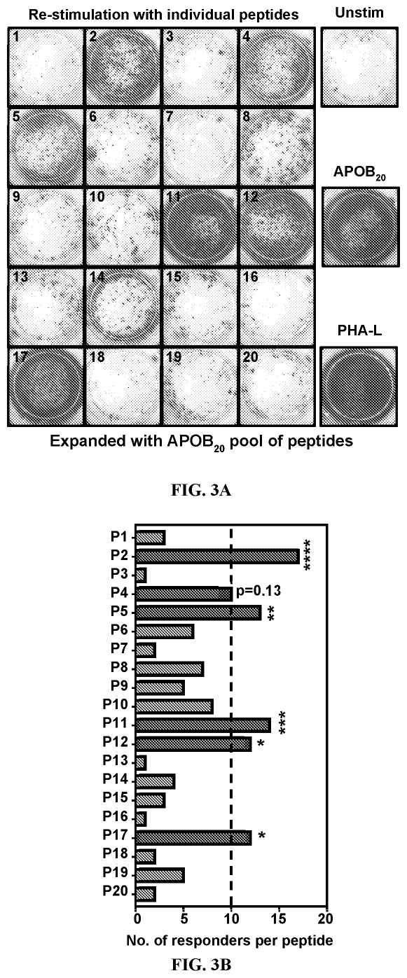

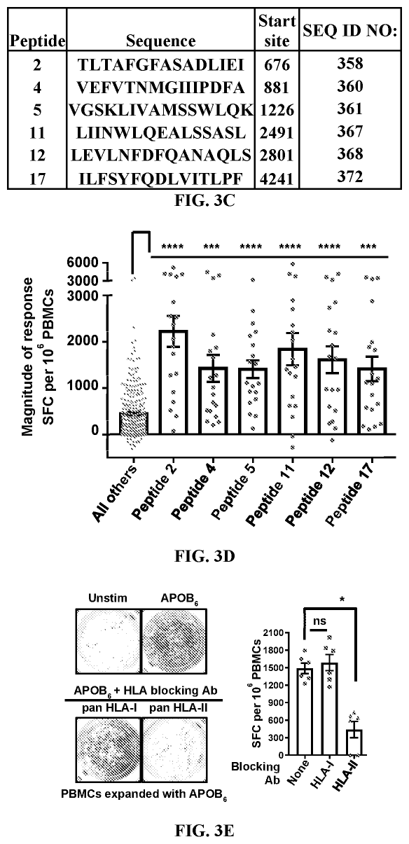

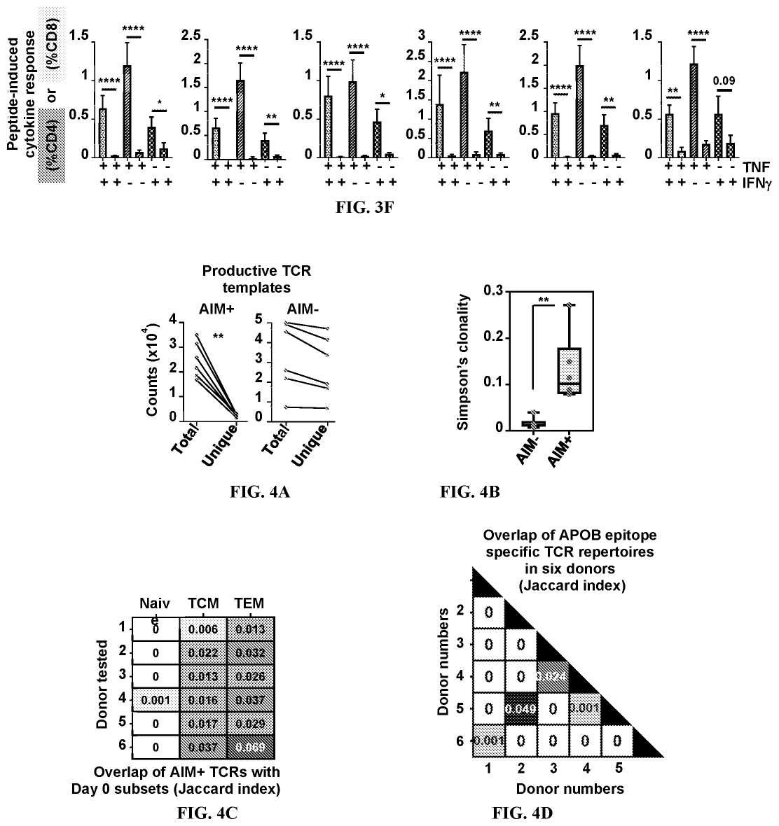

Discovery of immunodominant APOB-derived CD4 T cell epitopes. As all peptides with a capacity to bind HLA-II alleles do not exhibit comparable immunogenic potential, APOB-specific responses to single epitopes were determined. To do this, the inventors used the optimized IFNγ ELISpot assay (FIG. 1A) because Th1 cytokine response to APOB peptides is most robust (FIG. 9D) and easily detectable in high-throughput screens. PBMCs from HLA-typed donors (demographics and HLA-II alleles in Tables 5, 6) were incubated with the APOB₂₀ pool for 14 days with intermittent IL-2 feeding (schematic in FIG. 11A). Subsequent re-stimulation with individual peptides resulted in a mosaic pattern of T cell responses of varying magnitudes that were associated with specific epitopes (FIG. 3A). Restimulation with the APOB₂₀ pool and with PHA-L served as positive controls, and unstimulated cells were negative controls. For each donor, APOB peptide-specific restimulation conditions were run in triplicate wells and the average number of spot-forming cells (SFCs) per 10⁶ PBMCs was calculated. To determine responder frequencies, a positive response was defined based on established criteria set for detection of responses to other autoantigenic epitopes⁴. (1) ≥100 spot-forming cells per 10⁶ PBMCs; (2) stimulation index ≥2 (3) p≤0.01 by Student's t-test, mean response in stimulated vs unstimulated replicate wells. Using these criteria, six peptides (#2, 4, 5, 11, 12 and 17) were found to induce positive responses in ≥10 out of 19 donors tested (FIG. 3B). The average magnitude of response elicited by each of these six epitopes (FIG. 3C) was significantly higher than the average response to all the other epitope candidates (FIG. 3D). Scrambled versions of these six epitopes (FIG. 11B), elicited negligible responses in the deconvolution assay (FIG. 11C), confirming that only the original APOB peptides are recognized by human autoreactive T cells.

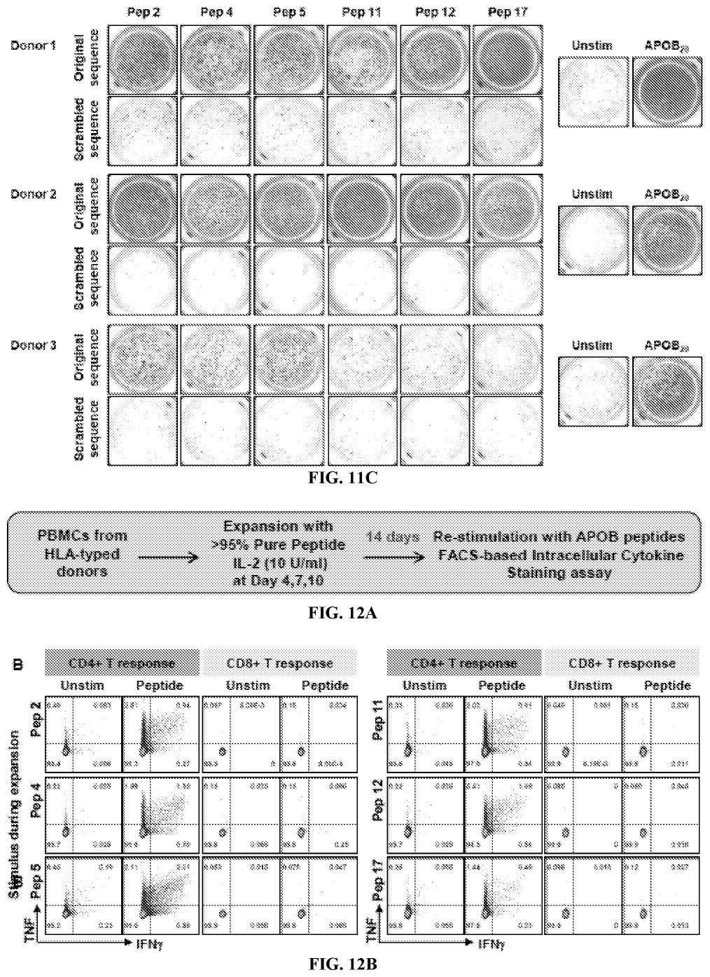

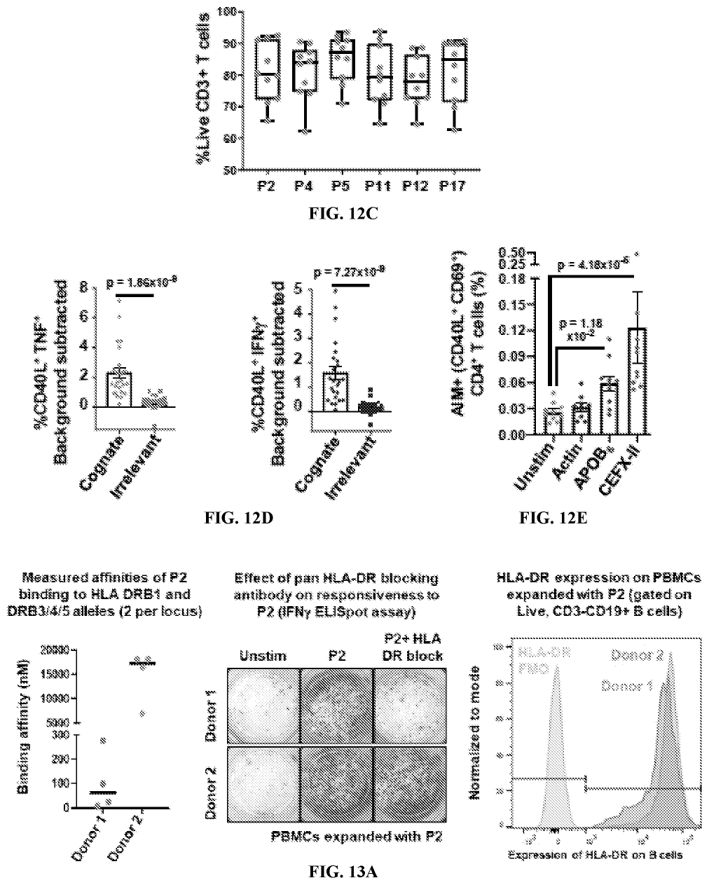

IFNγ responses to a combined pool of these HLA-II restricted dominant epitopes (APOB₆) were inhibited by pan-HLA-II blocking antibodies, but remained unaffected by pan-HLA-I blocking (FIG. 3E). Using HPLC-purified (>95% purity) peptides and flow cytometry-based ICS assays (schematic in FIG. 12A), the inventors validated that Th1 cytokine (TNF and IFNg) responses to all six epitopes are mediated by CD4⁺ T cells, with negligible CD8⁺ T response (FIG. 3F, FIG. 12B). None of the peptides induced any significant cell death in the expanded and restimulated T cells (FIG. 12C). Furthermore, only cognate epitopes, and not an irrelevant peptide, triggered Th1 responses in activated CD4⁺ T cells (FIG. 12D).

Evaluation of ex vivo responses using the 24 h AIM assay (FIGS. 2A to 2D) revealed significant increase in % CD40L⁺CD69⁺CD4⁺ T cells, as compared to unstimulated controls, in APOB₆ and CEFX-II stimulated PBMCs, but not in response to the actin pool (FIG. 12E). For accurate comparisons, all peptides were synthesized by the same manufacturer.

Next, the RATE (Restrictor Analysis Tool for Epitopes) tool (IEDB.org) was used to computationally infer putative HLA-II associations of the dominant peptides, based on observed positive responses (FIG. 3B) and HLA-II allele expression in the screening cohort (Table 6)^{39,40}. As expected, these APOB peptides, which were shown to bind multiple HLA-II alleles in the binding assay (FIG. 9A), did not exhibit significant positive association with any particular donor HLA-II allele.

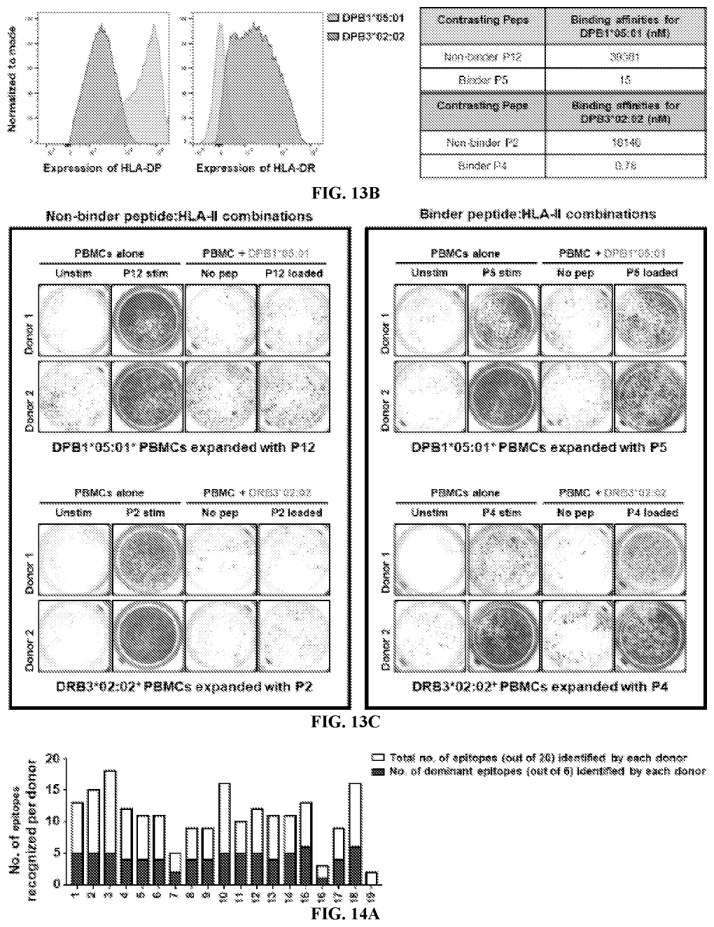

To further examine dependency of APOB peptide immunogenicity on their measured binding affinities and expression of binder alleles in the responding donors, case-control scenarios were constructed for binder and non-binder peptide-HLA-II combinations. First, the effect of pan-HLA-DR blockade on the immunogenicity of P2 in two donors was studied (FIG. 13A) whose alleles at DRB1 and DRB3/4/5 loci exhibited contrasting binding preferences for P2 (left). A pan-HLA-DR blocking antibody inhibited responses to P2 in donor 1 (middle), who expressed HLA-DR alleles with strong binding affinities for P2. Immunogenicity of the same peptide in donor 2, expressing DQ and DP binder alleles and DR non-binder alleles, remained unaffected by HLA-DR blockade (middle). Both donors expressed similar levels of HLA-DR molecules on B cells, the major antigen-presenting cells in peptide-expanded PBMCs (right).

In another strategy, single HLA-II transfected cell lines⁴¹ expressing either DPB1*05:01 or DRB3*02:02 (FIG. 13B, left) were used and examined the immunogenicity of two contrasting sets of binder and non-binder APOB peptides (FIG. 13B, right). It was found that exogenous addition of either binder or non-binder peptide elicited responses in PBMCs, because they express a combination of all HLA-II alleles present in that donor. However, under conditions where peptide presentation was limited to only one HLA-II molecule (DPB1*05:01 or DRB3*02:02 cell lines), it was found that the responses to each peptide were dependent on their binding preferences (FIG. 13C).

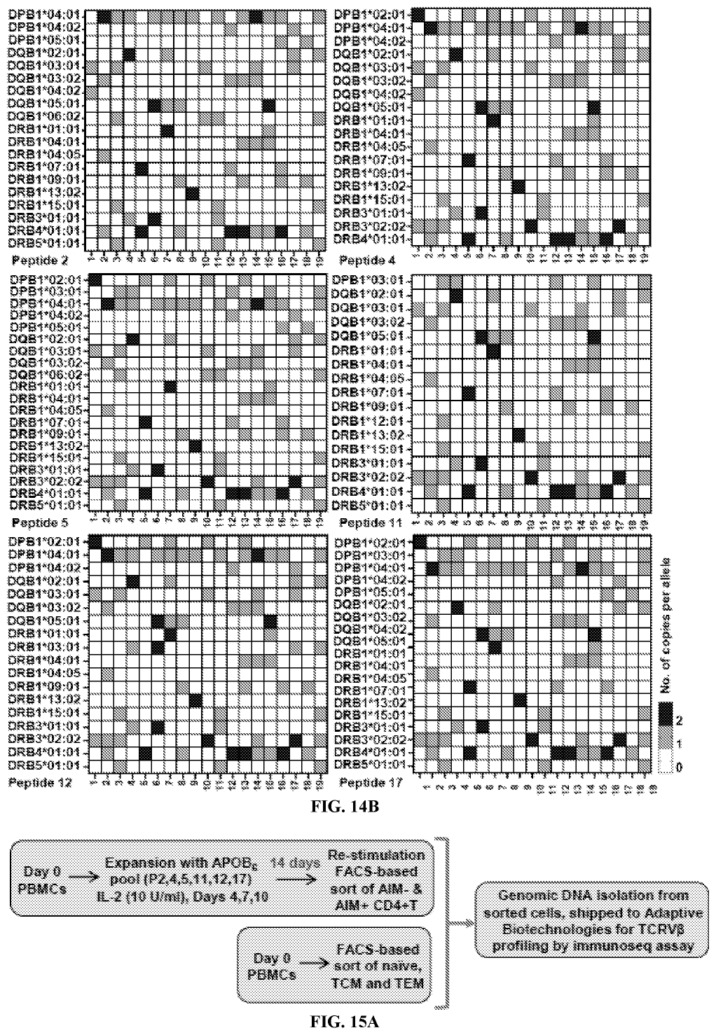

In these deconvolution experiments (FIG. 3B), 16 out of 19 donors identified at least 4 of the 6 dominant epitopes, with only one donor not responding to any of them (FIG. 14A). Every donor expressed at least one binder allele for each of the six peptides (FIG. 14B). Together these data (FIGS. 13A-13C, 14A-14B) show that although immunogenicity of dominant APOB epitopes relies on specific peptide-HLA-II binder combinations, a combined peptide pool of these six promiscuous dominant epitopes (APOB₆) will bind most of the frequently occurring HLA-II alleles (Table 1) and can allow evaluation of APOB-specific CD4⁺ T responses in a range of donors, irrespective of their HLA-II allelic heterogeneity.

Dominant APOB epitopes activate oligoclonal populations of APOB-reactive CD4⁺ T cells.

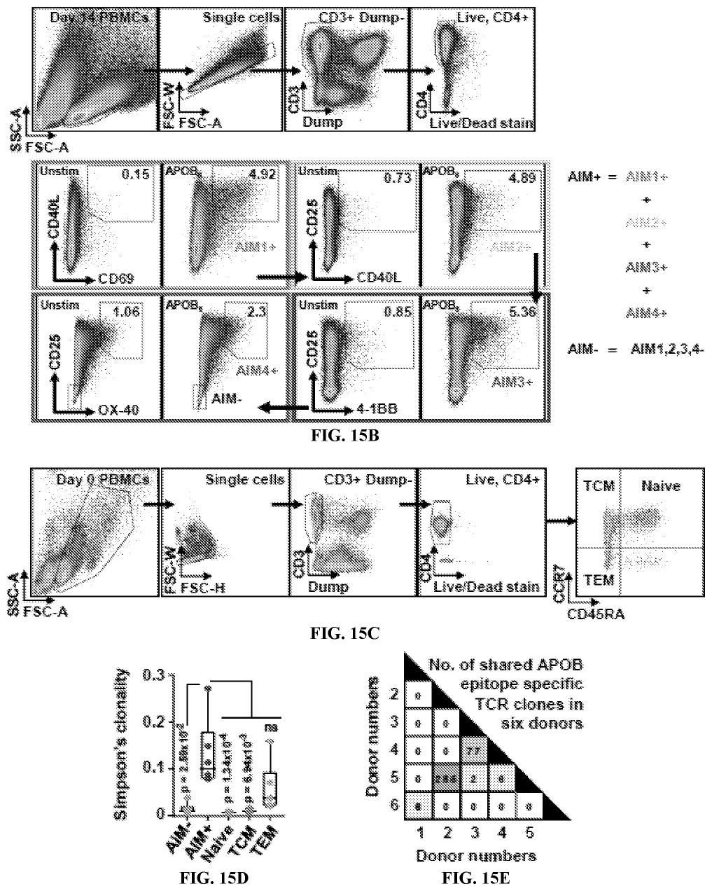

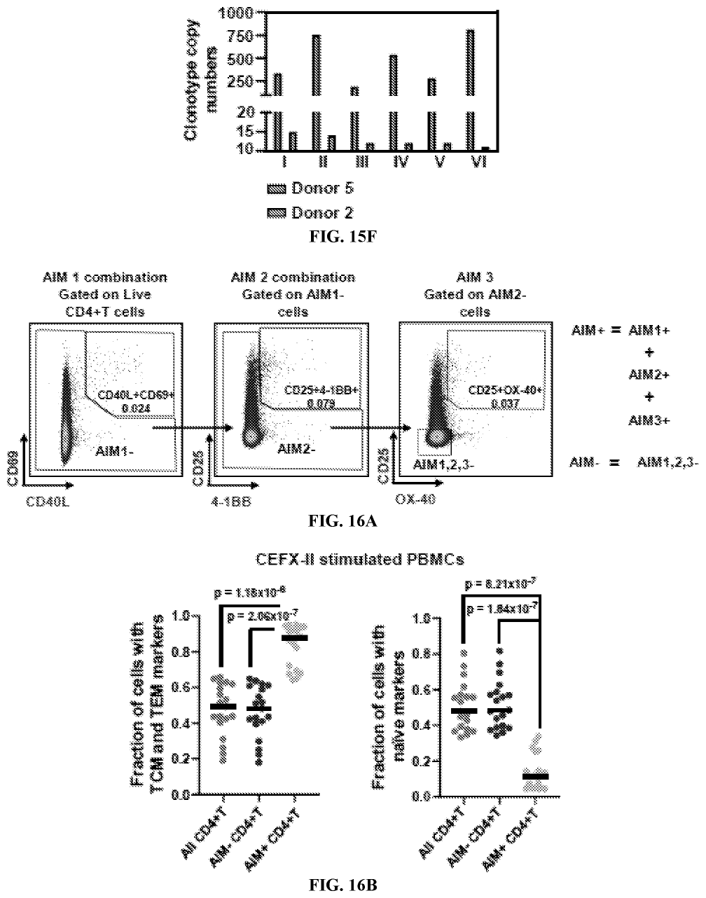

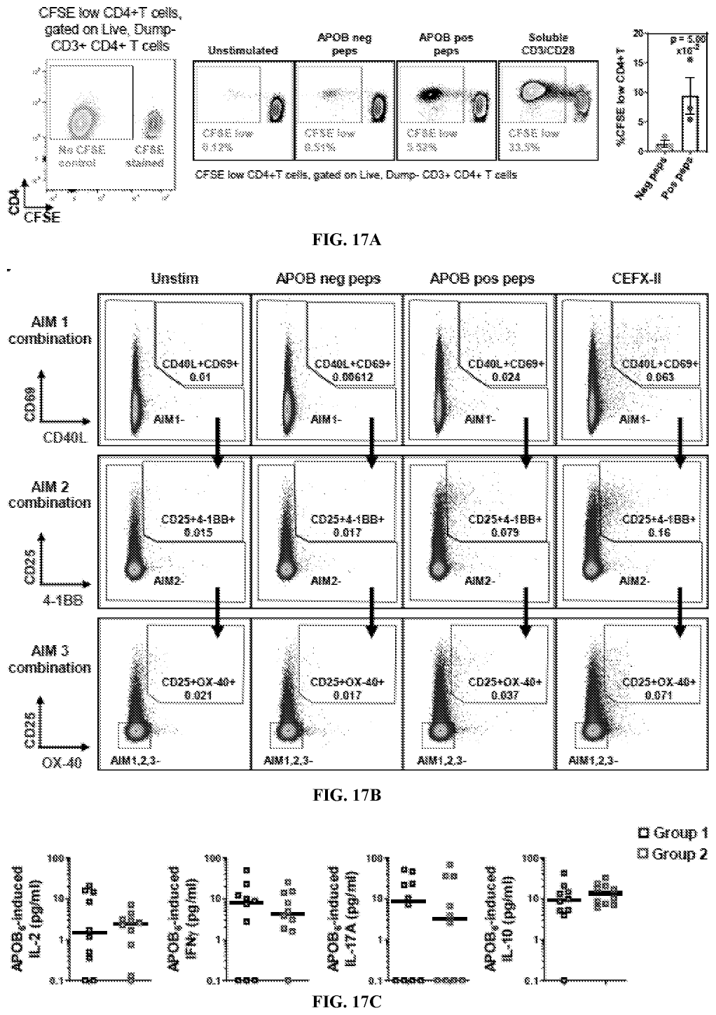

The TCR repertoire, diversity and clonality are important indicators of a T cell-mediated immunological reaction and reflect the immune status of an individual^{22,42}. To examine the clonotypic identities of autoreactive CD4⁺ T cells, the hypervariable TCRβ DR3 region was sequenced using a DNA-based sequencing technique⁴² (Adaptive Biotechnologies), which have been used to profile clonal populations of other self-antigen specific T cells⁴³. PBMCs from six HLA-typed donors (demographics and HLA-II alleles in Tables 5, 6) were expanded (14-days) and restimulated (24 h) with the APOB₆ pool. To isolate viable responder and non-responder CD4⁺ T cells from the same pool of stimulated PBMCs (schematic in FIG. 15A, gating strategy in FIG. 151B), the inventors used the AIM assay protocol (FIGS. 2A to 2D).

TABLE 6
HLA Class II alleles of donors in the screening cohort
Donors used in ELISpot-based deconvolution assay

| | | | | | |
|---|---|---|---|---|---|
| 1 | DPB1*02:01 | DPB1*02:01 | DQA1*04:01 | DQA1*05:01 | DQB1*03:01 |
| 2 | DPB1*04:01 | DPB1*04:01 | DQA1*01:03 | DQA1*03:01 | DQB1*03:02 |
| 3 | DPB1*03:01 | DPB1*04:01 | DQA1*01:02 | DQA1*05:01 | DQB1*03:01 |
| 4 | DPB1*03:01 | DPB1*04:01 | DQA1*04:01 | DQA1*05:01 | DQB1*02:01 |
| 5 | DPB1*02:01 | DPB1*17:01 | DQA1*01:02 | DQA1*02:01 | DQB1*06:04 |
| 6 | DPB1*01:01 | DPB1*04:01 | DQA1*01:01 | DQA1*01:01 | DQB1*05:01 |
| 7 | DPB1*02:01 | DPB1*04:01 | DQA1*05:01 | DQA1*05:01 | DQB1*02:01 |
| 8 | DPB1*04:01 | DPB1*11:01 | DQA1*01:01 | DQA1*03:01 | DQB1*03:03 |
| 9 | DPB1*04:01 | DPB1*17:01 | DQA1*01:02 | DQA1*01:02 | DQB1*06:04 |
| 10 | DPB1*02:01 | DPB1*18:01 | DQA1*01:02 | DQA1*05:01 | DQB1*03:01 |
| 11 | DPB1*04:01 | DPB1*13:01 | DQA1*01:02 | DQA1*01:03 | DQB1*06:02 |
| 12 | DPB1*03:01 | DPB1*04:02 | DQA1*02:01 | DQA1*03:01 | DQB1*02:02 |
| 13 | DPB1*02:01 | DPB1*04:01 | DQA1*03:01 | DQA1*03:01 | DQB1*03:02 |
| 14 | DPB1*04:01 | DPB1*04:01 | DQA1*03:01 | DQA1*05:01 | DQB1*03:01 |
| 15 | DPB1*03:01 | DPB1*04:01 | DQA1*01:01 | DQA1*03:01 | DQB1*05:01 |
| 16 | DPB1*04:01 | DPB1*05:01 | DQA1*02:01 | DQA1*03:01 | DQB1*02:02 |
| 17 | DPB1*02:02 | DPB1*04:02 | DQA1*05:01 | DQA1*05:01 | DQB1*02:01 |
| 18 | DPB1*05:01 | DPB1*13:01 | DQA1*01:03 | DQA1*03:01 | DQB1*03:03 |
| 19 | DPB1*03:01 | DPB1*04:01 | DQA1*01:02 | DQA1*05:01 | DQB1*02:01 |

Donors used in Peptide-specific ICS assay

| | | | | | |
|---|---|---|---|---|---|
| 1 | DPB1*03:01 | DPB1*04:01 | DQA1*04:01 | DQA1*05:01 | DQB1*02:01 |
| 2 | DPB1*04:01 | DPB1*11:01 | DQA1*01:01 | DQA1*03:01 | DQB1*03:03 |
| 3 | DPB1*04:01 | DPB1*13:01 | DQA1*01:02 | DQA1*01:03 | DQB1*06:02 |
| 4 | DPB1*02:01 | DPB1*04:01 | DQA1*03:01 | DQA1*03:01 | DQB1*03:02 |
| 5 | DPB1*04:01 | DPB1*04:01 | DQA1*03:01 | DQA1*05:01 | DQB1*03:01 |
| 6 | DPB1*03:01 | DPB1*04:01 | DQA1*01:01 | DQA1*03:01 | DQB1*05:01 |
| 7 | DPB1*04:01 | DPB1*05:01 | DQA1*02:01 | DQA1*03:01 | DQB1*02:02 |
| 8 | DPB1*03:01 | DPB1*04:01 | DQA1*01:02 | DQA1*03:01 | DQB1*03:03 |
| 9 | DPB1*04:01 | DPB1*13:01 | DQA1*01:01 | DQA1*01:01 | DQB1*05:01 |
| 10 | DPB1*02:01 | DPB1*02:01 | DQA1*01:02 | DQA1*05:01 | DQB1*03:01 |

Donors used in Immunoseq-based TCR profiling experiment

| | | | | | |
|---|---|---|---|---|---|
| 1 | DPB1*04:01 | DPB1*11:01 | DQA1*01:01 | DQA1*03:01 | DQB1*03:03 |
| 2 | DPB1*04:01 | DPB1*13:01 | DQA1*01:02 | DQA1*01:03 | DQB1*06:02 |
| 3 | DPB1*04:01 | DPB1*04:01 | DQA1*03:01 | DQA1*05:01 | DQB1*03:01 |
| 4 | DPB1*04:01 | DPB1*05:01 | DQA1*02:01 | DQA1*03:01 | DQB1*02:02 |
| 5 | DPB1*02:02 | DPB1*04:02 | DQA1*05:01 | DQA1*05:01 | DQB1*02:01 |
| 6 | DPB1*03:01 | DPB1*04:01 | DQA1*01:02 | DQA1*03:01 | DQB1*03:03 |

| Donors used in ELISpot-based deconvolution assay | | | | | |
|---|---|---|---|---|---|
| 1 | DQB1*04:02 | DRB1*08:11 | DRB1*11:01 | DRB3*02:02 | n/a |
| 2 | DQB1*06:03 | DRB1*04:05 | DRB1*13:01 | DRB3*02:02 | DRB4*01:01 |
| 3 | DQB1*06:02 | DRB1*12:01 | DRB1*15:01 | DRB3*02:02 | DRB5*01:01 |
| 4 | DQB1*02:01 | DRB1*03:01 | DRB1*08:01 | DRB3*01:01 | n/a |
| 5 | DQB1*06:04 | DRB1*07:01 | DRB1*07:01 | DRB4*01:01 | DRB4*01:01 |
| 6 | DQB1*05:01 | DRB1*03:01 | DRB1*03:01 | DRB3*01:01 | DRB3*01:01 |
| 7 | DQB1*05:01 | DRB1*01:01 | DRB1*01:01 | n/a | n/a |
| 8 | DQB1*05:01 | DRB1*09:01 | DRB1*10:01 | DRB4*01:01 | n/a |
| 9 | DQB1*06:04 | DRB1*13:02 | DRB1*13:02 | DRB3*03:01 | DRB3*03:01 |
| 10 | DQB1*06:02 | DRB1*11:01 | DRB1*11:02 | DRB3*02:02 | DRB3*02:02 |
| 11 | DQB1*06:03 | DRB1*13:01 | DRB1*15:01 | DRB3*01:01 | DRB5*01:01 |
| 12 | DQB1*03:02 | DRB1*04:03 | DRB1*07:01 | DRB4*01:01 | DRB4*01:01 |
| 13 | DQB1*03:03 | DRB1*04:01 | DRB1*09:01 | DRB4*01:01 | DRB4*01:01 |
| 14 | DQB1*03:02 | DRB1*04:01 | DRB1*11:01 | DRB3*02:02 | DRB4*01:01 |
| 15 | DQB1*05:01 | DRB1*01:01 | DRB1*04:01 | DRB4*01:01 | n/a |
| 16 | DQB1*03:03 | DRB1*07:01 | DRB1*09:01 | DRB4*01:01 | DRB4*01:01 |
| 17 | DQB1*03:01 | DRB1*03:01 | DRB1*11:02 | DRB3*02:02 | DRB3*02:02 |
| 18 | DQB1*06:01 | DRB1*08:03 | DRB1*09:01 | DRB4*01:01 | n/a |
| 19 | DQB1*06:02 | DRB1*03:01 | DRB1*15:01 | DRB3*02:02 | DRB5*01:01 |
| Donors used in Peptide-specific ICS assay | | | | | |
| 1 | DQB1*02:01 | DRB1*03:01 | DRB1*08:01 | DRB3*01:01 | n/a |
| 2 | DQB1*05:01 | DRB1*09:01 | DRB1*10:01 | DRB4*01:01 | n/a |
| 3 | DQB1*06:03 | DRB1*13:01 | DRB1*15:01 | DRB3*01:01 | DRB5*01:01 |
| 4 | DQB1*03:03 | DRB1*04:01 | DRB1*09:01 | DRB4*01:01 | DRB4*01:01 |
| 5 | DQB1*03:02 | DRB1*04:01 | DRB1*11:01 | DRB3*02:02 | DRB4*01:01 |
| 6 | DQB1*05:01 | DRB1*01:01 | DRB1*04:01 | DRB4*01:01 | n/a |
| 7 | DQB1*03:03 | DRB1*07:01 | DRB1*09:01 | DRB4*01:01 | DRB4*01:01 |
| 8 | DQB1*06:02 | DRB1*09:01 | DRB1*15:01 | DRB4*01:01 | DRB5*01:01 |
| 9 | DQB1*05:01 | DRB1*01:01 | DRB1*01:01 | n/a | n/a |
| 10 | DQB1*06:02 | DRB1*11:04 | DRB1*15:01 | DRB3*02:02 | DRB5*01:01 |
| Donors used in Immunoseq-based TCR profiling experiment | | | | | |
| 1 | DQB1*05:01 | DRB1*09:01 | DRB1*10:01 | DRB4*01:01 | n/a |
| 2 | DQB1*06:03 | DRB1*13:01 | DRB1*15:01 | DRB3*01:01 | DRB5*01:01 |
| 3 | DQB1*03:02 | DRB1*04:01 | DRB1*11:01 | DRB3*02:02 | DRB4*01:01 |
| 4 | DQB1*03:03 | DRB1*07:01 | DRB1*09:01 | DRB4*01:01 | DRB4*01:01 |
| 5 | DQB1*03:01 | DRB1*03:01 | DRB1*11:02 | DRB3*02:02 | DRB3*02:02 |
| 6 | DQB1*06:02 | DRB1*09:01 | DRB1*15:01 | DRB4*01:01 | DRB5*01:01 |

The surface expression of five most commonly used activation markers—CD4L, CD69, CD25, OX-40 and 4-1BB, was assessed, which maximized detection of APOB-induced activated (AIM⁺) CD4⁺ T cell repertoire, irrespective of their cytokine secretion potential. AIM⁻ CD4⁺ T cells, that did not express any of the activation markers tested, were used as “non-responder” negative controls (gating strategy in FIG. 15B). As a second control, unexpanded PBMCs (day 0 samples), sorted into naïve, central memory (TCM) and effector memory (TEM) CD4⁺ T cells, were used (gating strategy in FIG. 15C). This allowed tracing the APOB₆-specific AIM⁺ T cell clones in naïve and memory T cell lineages. Genomic DNA, isolated from sorted AIM⁻ and AIM⁺ Day 14 samples and naïve, TCM and TEM Day 0 samples from six donors, was used as the starting material for TCRVβ sequencing (schematic in FIG. 15A) using an optimized two-step, amplification bias-controlled multiplex-PCR-based approach^{42,44} (immunoSEQ Human TCRB Assay, Adaptive Biotechnologies).

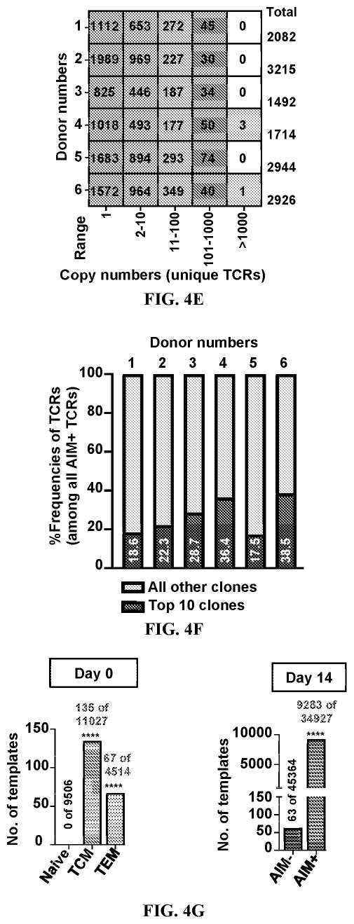

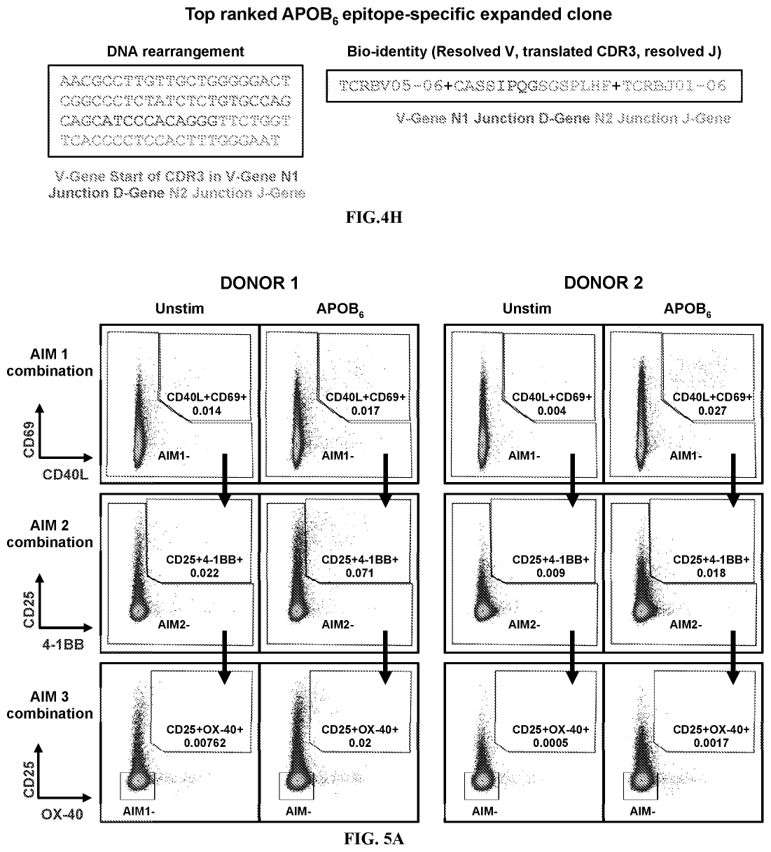

As most T cells encode only one somatically rearranged TCR⁴⁵, frequencies of CDR3 templates identified from genomic DNA strongly correlate with the relative abundances of the clonotypic T cells bearing that CDR3 sequence⁴⁶. For this analysis, the inventors focused on productive templates in which the nucleotide sequence of the CDR3 region is in-frame for protein translation and does not contain any stop codons. A comparison of total vs unique template counts (details in FIGS. 15A to 15F) detected in AIM⁺ and AIM⁻ cells revealed an enrichment of a restricted set of unique rearrangements in the responding CD4⁺ T cells (FIG. 4A), indicating APOB-dependent selection of specific TCR clones.

TABLE 7
Sample details for Day 0 and Day 14 APOB stimulated sets

| | Input | Total | |
|---|---|---|---|
| | amount | productive | Unique |
| | (ng) | templates | rearrangements |
| Day 14_AIM (−) | | | |
| Donor 1 | 400.388 | 49956 | 46895 |
| Donor 2 | 72.412 | 7206 | 6731 |
| Donor 3 | 179.588 | 21770 | 16762 |
| Donor 4 | 221.588 | 25928 | 18974 |
| Donor 5 | 400.332 | 48987 | 41250 |
| Donor 6 | 392.484 | 45364 | 33615 |
| Day 14_AIM (+) | | | |
| Donor 1 | 202.76 | 21697 | 2082 |
| Donor 2 | 189.724 | 18591 | 3215 |
| Donor 3 | 179.588 | 16700 | 1492 |
| Donor 4 | 357.724 | 25796 | 1741 |
| Donor 5 | 327.312 | 31354 | 2944 |
| Donor 6 | 400.652 | 34927 | 2926 |
| Day 0_Naive | | | |
| Donor 1 | 217.24 | 30844 | 29599 |
| Donor 2 | 108.62 | 14606 | 14177 |
| Donor 3 | 146.276 | 20152 | 19594 |
| Donor 4 | 196.964 | 25982 | 25351 |
| Donor 5 | 107.172 | 13665 | 13204 |
| Donor 6 | 107.172 | 9506 | 9224 |
| Day 0_TCM | | | |
| Donor 1 | 215.792 | 21331 | 18637 |
| Donor 2 | 118.76 | 15076 | 13188 |
| Donor 3 | 102.828 | 13933 | 11867 |
| Donor 4 | 127.448 | 14966 | 12432 |
| Donor 5 | 114.412 | 14668 | 12681 |
| Donor 6 | 91.24 | 11027 | 9306 |
| Day 0_TEM | | | |
| Donor 1 | 37.656 | 2457 | 1853 |
| Donor 2 | 69.516 | 5889 | 4506 |
| Donor 3 | 47.792 | 6218 | 4245 |
| Donor 4 | 82.552 | 7937 | 3325 |
| Donor 5 | 28.964 | 2824 | 1935 |
| Donor 6 | 39.104 | 4514 | 2855 |

The inventors determined 14,400 unique TCR rearrangements from a total of 149,065 productive TCRβ chain CDR3 templates identified in APOB-specific CD4⁺ T cells that recognized these six immunodominant epitopes. Comparison of the repertoire characteristics of AIM⁻ and AIM⁺ cells using diversity and clonality metrics⁴⁷ (FIG. 4B), confirmed that AIM⁺ populations are represented by fewer but more abundant unique clonotypes (higher average Simpson's clonality). The APOB-AIM⁺CD4⁺ T cells, and not the AIM⁻CD4⁺ T cells, were significantly more oligoclonal than all Day 0 subsets (FIG. 15D), thereby validating the efficacy of the expansion and restimulation regime in detecting epitope-specific oligoclonal CD4⁺ T cells in a polyclonal pool of PBMCs. While expansion improves sensitivity, restimulation enforces specificity. Comparing the extent of similarity between APOB-specific AIM⁺ TCR repertoire and the different ex vivo subsets using Jaccard's index⁴⁷ (FIG. 4C), revealed that the APOB-specific TCR repertoire exhibited some overlap with central and effector memory CD4⁺ T cell TCRs, but not with naïve populations, suggesting that exposure to these epitopes and memory formation had occurred in vivo.

To assess whether shared TCR clones appear across AIM⁺ T cells from different donors, the inventors examined repertoire overlap (FIG. 4D) and performed Venn diagram analysis of the TCR rearrangements (FIG. 15E). It was found that most clones that were shared between two donors showed preferential expansion in one donor and were singletons (one copy) or rare (<10 copy numbers) in the other donor. Only 6 clones, shared between donors 2 and 5, were present in >10 copy numbers in both donors (FIG. 15F). These data show that CD4⁺ T responses to APOB₆ consists predominantly of private TCR clones restricted to individual donors. Analysis of the extent of APOB₆ driven expansion of clonotypes within each donor showed that almost half of the AIM⁺ rearrangements were present at >1 copy number, with enrichments ranging from low (2-10) to moderate (11-100) to high (101-1000) (FIG. 4E). In two donors, APOB-specific clones with >1000 copies were detected (FIG. 4E). As the frequencies of the top 10 most abundant clones in each donor accounted for a significant fraction (frequencies 18-39%; average 270%) of all TCR templates (FIG. 4F), the sequence identities (Table 8) of these clones were examined in greater detail. The inventors verified their expansion over AIM⁻ controls by examining clone-specific relative frequencies using Fisher's exact test and odds ratio analysis (Table 8).

TABLE 8
Details of top 10 TCR rearrangements detected in AIM+ CD4+T cells from individual
donors.
Donor 1

| | No. of clonE−specific | | FDR | |
|---|---|---|---|---|
| | productive templates | | adjusted | |
| | AIM⁻ CD4⁺ | AIM⁺ CD4⁺ | p values | |
| | T | T | Fisher's | Log₁₀ |
| Bio-identity | (Total | (Total | exact | Odds |
| (CDR3 AA, resolved VJ genes) | 49956) | 21697) | test | Ratio |
| CASSTTGLAAYNEQFF (SEQ ID | 13 | 519 | 1.74E−247 | 1.97 |
| NO: 190), +TCRBV19-01+TCRBJ02-01 | | | | |
| CASSFVDSPESYEQYF (SEQ ID | 11 | 486 | 1.94E−233 | 2.01 |
| NO: 191), +TCRBV12-X+TCRBJ02-07 | | | | |
| CASSLTRIPEQYF (SEQ ID | 136 | 459 | 1.63E−123 | 0.90 |
| NO: 192),+TCRBV28-01+TCRBJ02-07 | | | | |
| CSARVTGGGAHTGELFF (SEQ ID | 2 | 422 | 1.39E−215 | 2.69 |
| NO: 193), +TCRBV20-X+TCRBJ02-02 | | | | |
| CASSLALGTGDTEAFF (SEQ ID | 1 | 410 | 1.55E−211 | 2.98 |
| NO: 194), +TCRBV05-01+TCRBJ01-01 | | | | |
| CASSLAGTGTNEQFF (SEQ ID NO: 195), | 1 | 396 | 2.89E−204 | 2.97 |
| +TCRBV05-01+TCRBJ02-01 | | | | |
| CASRGQRDTGELFF (SEQ ID NO: 196), | 2 | 367 | 4.9E−187 | 2.63 |
| +TCRBV19-01+TCRBJ02-02 | | | | |
| CASSIRPNDEQFF (SEQ ID NO: 197), | 1 | 366 | 1.34E−188 | 2.93 |
| +TCRBV09-01+TCRBJ02-01 | | | | |
| CASSYSSGGGNEQFF (SEQ ID NO: 198), | 2 | 348 | 3.75E−177 | 2.61 |
| +TCRBV06-05+TCRBJ02-01 | | | | |
| CASRTIGQGSFNYGYTF (SEQ ID | 1 | 257 | 5.35E−132 | 2.78 |
| NO: 199), +TCRBV27-01+TCRBJ01-02 | | | | |

Donor 2

| | No. of clonE−specific | | | |
|---|---|---|---|---|
| | productive templates | | FDR | |
| | AIM⁻ CD4⁺ | AIM⁺ CD4⁺ | adjusted | |
| | T | T | p values | Log₁₀ |
| Bio-identity | (Total | (Total | Fisher's | Odds |
| (CDR3 AA, resolved VJ genes) | 7206) | 18591) | exact test | Ratio |
| CASSSLTPEAYGYTF (SEQ ID NO: 200), | 2 | 796 | 1.69E−111 | 2.21 |
| +TCRBV28-01+TCRBJ01-02 | | | | |
| CSAMSPSGTGELFF (SEQ ID NO: 201), | 0 | 549 | 9.47E−80 | 12.19 |
| +TCRBV20-X+TCRBJ02-02 | | | | |
| CASSDDLAANVLTF (SEQ ID NO: 202), | 4 | 411 | 8.59E−53 | 1.61 |
| +TCRBV13-01+TCRBJ02-06 | | | | |
| CASSPRAGGAVREQYF (SEQ ID | 0 | 375 | 1.81E−54 | 12.03 |
| NO: 203), +TCRBV05-08+TCRBJ02-07 | | | | |
| CAWSVGGPGANSPLHF (SEQ ID | 10 | 369 | 6.38E−40 | 1.16 |
| NO: 204), +TCRBV30-01+TCRBJ01-06 | | | | |
| CASSKVNEKLFF (SEQ ID NO: 205), | 9 | 361 | 6.85E−40 | 1.19 |
| +TCRBV03-01/03-02+TCRBJ01-04 | | | | |
| CASSLGGRSNQPQHF (SEQ ID NO: 206), | 0 | 359 | 3.53E−52 | 12.01 |
| +TCRBV05-01+TCRBJ01-05 | | | | |
| CSAREPHDTSTDTQYF (SEQ ID | 0 | 338 | 3.77E−49 | 11.98 |
| NO: 207), +TCRBV20-X+TCRBJ02-03 | | | | |
| CSASRTYDNQPQHF (SEQ ID NO: 208), | 0 | 326 | 2.00E−47 | 11.96 |
| +TCRBV20-01+TCRBJ01-05 | | | | |
| CASSWDRTKFGNQPQHF (SEQ ID | 1 | 268 | 3.32E−37 | 2.02 |
| NO: 209), +TCRBV19-01+TCRBJ01-05 | | | | |

Donor 3

| | No. of clonE−specific | | | |
|---|---|---|---|---|
| | productive templates | | FDR | |
| | AIM⁻ CD4⁺ | AIM⁺ CD4⁺ | adjusted | |
| | T | T | p values | Log₁₀ |
| Bio-identity | (Total | (Total | Fisher's | Odds |
| (CDR3 AA, resolved VJ genes) | 21770) | 16700) | exact test | Ratio |
| CSARADGQGYGYTF (SEQ ID NO: 342), | 26 | 996 | 0 | 1.73 |
| +TCRBV20-X+TCRBJ01-02 | | | | |
| CASSQEVGGDHGYTF (SEQ ID | 4 | 759 | 4.73E−270 | 2.41 |
| NO: 343), +TCRBV04-03+TCRBJ01-02 | | | | |
| CASSSSGSNYNEQFF (SEQ ID NO: 210), | 52 | 597 | 1.63E−154 | 1.19 |
| +TCRBV03-01/03-02+TCRBJ02-01 | | | | |
| CASIRPGLNYGYTF (SEQ ID NO: 344), | 48 | 436 | 6.78E−105 | 1.08 |
| +TCRBV06-05+TCRBJ01-02 | | | | |
| CASSPADREETQYF (SEQ ID NO: 345), | 56 | 410 | 6.06E−91 | 0.99 |
| +TCRBV18-01+TCRBJ02-05 | | | | |
| CSARWYNEQFF (SEQ ID NO: 211), | 7 | 349 | 1.78E−115 | 1.82 |
| +TCRBV20-01+TCRBJ02-01 | | | | |
| CATSEPGPLNQPQHF (SEQ ID NO: 346), | 11 | 348 | 7.96E−110 | 1.62 |
| +TCRBV24-01+TCRBJ01-05 | | | | |
| CASSPLLYSGNTIYF (SEQ ID NO: 212), | 0 | 347 | 3.98E−127 | 12.09 |
| +TCRBV07-02+TCRBJ01-03 | | | | |
| CASRTTTGFLSPLHF (SEQ ID | 5 | 273 | 3.31E−91 | 1.86 |
| NO: 347),+TCRBV27-01+TCRBJ01-06 | | | | |
| CASSLPRDRDYGYTF (SEQ ID | 1 | 269 | 1.91E−96 | 2.55 |
| NO: 213), +TCRBV06-02+TCRBJ01-02 | | | | |

Donor 4

| | No. of clonE−specific | | | |
|---|---|---|---|---|
| | productive templates | | FDR | |
| | AIM⁻ CD4⁺ | AIM⁺ CD4⁺ | adjusted | |
| | T | T | p values | Log₁₀ |
| Bio-identity | (Total | (Total | Fisher's | Odds |
| (CDR3 AA, resolved VJ genes) | 25928) | 25796) | exact test | Ratio |
| CASSELGLNTDTQYF (SEQ ID | 190 | 2236 | 0 | 1.11 |
| NO: 214), +TCRBV06-01+TCRBJ02-03 | | | | |
| CASSAMVEQPQHF (SEQ ID NO: 215), | 503 | 1709 | 1.58E−160 | 0.55 |
| +TCRBV06-X+TCRBJ01-05 | | | | |
| CASSEMGLNTDTQYF (SEQ ID | 601 | 1601 | 5.93E−110 | 0.45 |
| NO: 216), +TCRBV06-01+TCRBJ02-03 | | | | |
| CASSPRGAVNTEAFF (SEQ ID | 130 | 950 | 3.33E−158 | 0.88 |
| NO: 217), +TCRBV07-09+TCRBJ01-01 | | | | |
| CASSELGTRPNEQFF (SEQ ID | 1 | 558 | 9.31E−168 | 2.76 |
| NO: 218), +TCRBV10-01+TCRBJ02-01 | | | | |
| CASRKQGLGYTEAFF (SEQ ID | 2 | 544 | 2.28E−161 | 2.45 |
| NO: 219), +TCRBV02-01+TCRBJ01-01 | | | | |
| CASSYRQTNQPQHF (SEQ ID NO: 220), | 0 | 509 | 2.75E−155 | 12.14 |
| +TCRBV06-02/06-03+TCRBJ01-05 | | | | |
| CSVLQHEQYF (SEQ ID NO: 221), | 153 | 466 | 9.39E−39 | 0.49 |
| +TCRBV29-01+TCRBJ02-07 | | | | |
| CASSPQPSTDTQYF (SEQ ID NO: 222), | 11 | 431 | 2.35E−113 | 1.60 |
| +TCRBV07-08+TCRBJ02-03 | | | | |
| CASSVRDYSPLHF (SEQ ID NO: 223), | 6 | 390 | 3.93E−108 | 1.82 |
| +TCRBV07-08+TCRBJ01-06 | | | | |

Donor 5

| | No. of clonE−specific | | | |
|---|---|---|---|---|
| | productive templates | | FDR | |
| | AIM⁻ CD4⁺ | AIM⁺ CD4⁺ | adjusted | |
| | T | T | p values | Log₁₀ |
| Bio-identity | (Total | (Total | Fisher's | Odds |
| (CDR3 AA, resolved VJ genes) | 48987) | 31354) | exact test | Ratio |
| CASSYGGGPPDTQYF (SEQ ID | 6 | 810 | 4.24E−320 | 2.34 |
| NO: 180), +TCRBV06-06+TCRBJ02-03 | | | | |
| CASSQTALYSGNTIYF (SEQ ID | 0 | 777 | 8.25E−320 | 12.08 |
| NO: 181), +TCRBV19-01+TCRBJ01-03 | | | | |
| CATALAGGHEQYF (SEQ ID NO: 182), | 3 | 753 | 1.07E−302 | 2.60 |
| +TCRBV06-05+TCRBJ02-07 | | | | |
| CSAREGALDNSPLHF (SEQ ID | 4 | 537 | 5.00E−212 | 2.33 |
| NO: 183), +TCRBV20-X+TCRBJ01-06 | | | | |
| CATSDFGKEGDEKLFF (SEQ ID | 2 | 518 | 3.10E−208 | 2.61 |
| NO: 184), +TCRBV24-01+TCRBJ01-04 | | | | |
| CASSFTYDEQFF (SEQ ID NO: 185), | 16 | 457 | 5.71E−162 | 1.66 |
| +TCRBV07-09+TCRBJ02-01 | | | | |
| CASSPGREPGNTIYF (SEQ ID | 179 | 422 | 2.52E−54 | 0.57 |
| NO: 186), +TCRBV07-09+TCRBJ01-03 | | | | |
| CASTATESSPLHF (SEQ ID NO: 187), | 0 | 415 | 1.45E−170 | 11.80 |
| +TCRBV12-03/12-04+TCRBJ01-06 | | | | |
| CASRKTGSLYGYTF (SEQ ID | 0 | 411 | 6.13E−169 | 11.80 |
| NO: 188), +TCRBV27-01+TCRBJ01-02 | | | | |
| CASSLRTGIINEQFF (SEQ ID NO: 189), | 0 | 383 | 1.64E−157 | 11.77 |
| +TCRBV05-01+TCRBJ02-01 | | | | |

Donor 6

| | No. of clonE−specific | | | |
|---|---|---|---|---|
| | productive templates | | FDR | |
| | AIM⁻ CD4⁺ | AIM⁺ CD4⁺ | adjusted | |
| | T | T | p values | Log₁₀ |
| Bio-identity | (Total | (Total | Fisher's | Odds |
| (CDR3 AA, resolved VJ genes) | 45364) | 34927) | exact test | Ratio |
| CASSIPQGSGSPLHF (SEQ ID NO: 179) | 63 | 9283 | 0 | 2.41 |
| +TCRBV05-06+TCRBJ01-06 | | | | |
| CASSLERGAYGYTF (SEQ ID NO: 224) | 2 | 963 | 0 | 2.81 |
| +TCRBV07-02+TCRBJ01-02 | | | | |
| CATSQGVGSGANVLTF (SEQ ID | 5 | 649 | 2.52E−225 | 2.24 |
| NO: 225), +TCRBV15-01+TCRBJ02-06 | | | | |
| CSATGGTNEKLFF (SEQ ID NO: 226) | 17 | 500 | 9.66E−155 | 1.59 |
| +TCRBV20-X+TCRBJ01-04 | | | | |
| CASRETGGAGELFF (SEQ ID NO: 227) | 1 | 393 | 1.05E−140 | 2.71 |
| +TCRBV06-05+TCRBJ02-02 | | | | |
| CASSISLALIYEQYF (SEQ ID NO: 228) | 4 | 371 | 3.85E−127 | 2.09 |
| +TCRBV19-01+TCRBJ02-07 | | | | |
| CASSLYAGQNTEAFF (SEQ ID | 2 | 344 | 5.41E−121 | 2.35 |
| NO: 229), +TCRBV07-02+TCRBJ01-01 | | | | |
| CASSPDFSGANVLTF (SEQ ID NO: 230) | 3 | 339 | 2.19E−117 | 2.17 |
| +TCRBV05-06+TCRBJ02-06 | | | | |
| CASSQTGQGYNEQFF (SEQ ID | 13 | 308 | 8.39E−93 | 1.49 |
| NO: 231), +TCRBV23-01+TCRBJ02-01 | | | | |
| CATSEGGRGRGPGELFF (SEQ ID | 1 | 292 | 1.84E−103 | 2.58 |
| NO: 232), +TCRBV24-01+TCRBJ02-02 | | | | |

For donors having a control group of TCR sequences from CEFX-1 stimulated PBMCs, the frequencies of top 10 APOB and top 10 CEFX-II TCRs were compared (Table 10) in APOB and CEFX-II-specific AIM⁺ subsets. Most TCRs were either highly enriched (having ˜10-fold higher productive frequencies) or found exclusively in any one of the AIM⁺ groups. Only 3 of the top clones (found in donors 2 and 5) occurred at comparable frequencies in the two AIM⁺CD4⁺ T subsets (Table 9). These findings show the degree of TCR cross-reactivity between APOB-specific CD4⁺ T cells and some CEFX-11 viral or bacterial antigen-specific CD4⁺ T cells may exist in some donors.

TABLE 10
Comparison of productive frequencies of APOB6 and CEFX-II
top 10 TCR rearrangements in APOB6 vs CEFX-II stimulated
AIM+ CD4 + T cells from individual donors.

| | | |
|---|---|---|
| | Frequency among | Frequency among |
| | all APOB AIM+ | all CEFX-II |
| | productive | AIM+ productive |
| TCR rank | TCR templates | TCR templates |

Top 10 APOB clones in Donor 1

| | | |
|---|---|---|
| APOB_1 | 0.024 | 0 |
| APOB_2 | 0.022 | 0 |
| APOB_3 | 0.021 | 0 |
| APOB_4 | 0.02 | 0 |
| APOB_5 | 0.019 | 0 |
| APOB_6 | 0.018 | 0 |
| APOB_7 | 0.017 | 0 |
| APOB_8 | 0.017 | 0 |
| APOB_9 | 0.016 | 0 |
| APOB_10 | 0.012 | 0 |

Top 10 CEFX-II clones in Donor 1

| | | |
|---|---|---|
| CEFX-II_1 | 0 | 0.05 |
| CEFX-II_2 | 0 | 0.04 |
| CEFX-II_3 | 0 | 0.02 |
| CEFX-II_4 | 0 | 0.018 |
| CEFX-II_5 | 0 | 0.018 |
| CEFX-II_6 | 0 | 0.015 |
| CEFX-II_7 | 0 | 0.015 |
| CEFX-II_8 | 0 | 0.015 |
| CEFX-II_9 | 0 | 0.013 |
| CEFX-II_10 | 0 | 0.013 |

Top 10 APOB clones in Donor 2

| | | |
|---|---|---|
| APOB_1 | 0.043 | 0 |
| APOB_2 | 0.03 | 0 |
| APOB_3 | 0.022 | 0.0013 |
| APOB_4 | 0.02 | 0 |
| APOB_5 | 0.02 | 0 |
| APOB_6 | 0.0194 | 0 |
| APOB_7 | 0.0193 | 0 |
| APOB_8 | 0.018 | 0.03 |
| APOB_9 | 0.0175 | 0 |
| APOB_10 | 0.0144 | 0 |

Top 10 CEFX-II clones in Donor 2

| | | |
|---|---|---|
| CEFX-II_1 | 0 | 0.052 |
| CEFX-II_2 | 0.018 | 0.03 |
| CEFX-II_3 | 0 | 0.029 |
| CEFX-II_4 | 0 | 0.0265 |
| CEFX-II_5 | 0.0136 | 0.024 |
| CEFX-II_6 | 0 | 0.021 |
| CEFX-II_7 | 0 | 0.0204 |
| CEFX-II_8 | 0 | 0.018 |
| CEFX-II_9 | 0 | 0.018 |
| CEFX-II_10 | 0 | 0.017 |

Top 10 APOB clones in Donor 3

| | | |
|---|---|---|
| APOB_1 | 0.06 | 0 |
| APOB_2 | 0.045 | 0 |
| APOB_3 | 0.036 | 0 |
| APOB_4 | 0.026 | 0 |
| APOB_5 | 0.025 | 0 |
| APOB_6 | 0.021 | 0 |
| APOB_7 | 0.021 | 0 |
| APOB_8 | 0.021 | 0 |
| APOB_9 | 0.016 | 0 |
| APOB_10 | 0.016 | 0 |

Top 10 CEFX-II clones in Donor 3

| | | |
|---|---|---|
| CEFX-II_1 | 0 | 0.06 |
| CEFX-II_2 | 0 | 0.054 |
| CEFX-II_3 | 0 | 0.045 |
| CEFX-II_4 | 0 | 0.036 |
| CEFX-II_5 | 0 | 0.028 |
| CEFX-II_6 | 0 | 0.021 |
| CEFX-II_7 | 0 | 0.017 |
| CEFX-II_8 | 0 | 0.015 |
| CEFX-II_9 | 0 | 0.013 |
| CEFX-II_10 | 0 | 0.011 |

Top 10 APOB clones in Donor 4

| | | |
|---|---|---|
| APOB_1 | 0.087 | 0 |
| APOB_2 | 0.066 | 0.007 |
| APOB_3 | 0.062 | 0 |
| APOB_4 | 0.037 | 0 |
| APOB_5 | 0.022 | 0 |
| APOB_6 | 0.021 | 0 |
| APOB_7 | 0.02 | 0 |
| APOB_8 | 0.018 | 0.002 |
| APOB_9 | 0.017 | 0 |
| APOB_10 | 0.015 | 0 |

Top 10 CEFX-II clones in Donor 4

| | | |
|---|---|---|
| CEFX-II_1 | 0 | 0.14 |
| CEFX-II_2 | 0 | 0.10 |
| CEFX-II_3 | 0 | 0.043 |
| CEFX-II_4 | 0 | 0.037 |
| CEFX-II_5 | 0 | 0.03 |
| CEFX-II_6 | 0 | 0.027 |
| CEFX-II_7 | 0 | 0.026 |
| CEFX-II_8 | 0 | 0.016 |
| CEFX-II_9 | 0 | 0.016 |
| CEFX-II_10 | 0 | 0.0154 |

Top 10 APOB clones in Donor 5

| | | |
|---|---|---|
| APOB_1 | 0.026 | 0 |
| APOB_2 | 0.025 | 0 |
| APOB_3 | 0.024 | 0 |
| APOB_4 | 0.017 | 0 |
| APOB_5 | 0.0165 | 0 |
| APOB_6 | 0.015 | 0 |
| APOB_7 | 0.0135 | 0.0006 |
| APOB_8 | 0.0132 | 0 |
| APOB_9 | 0.0131 | 0 |
| APOB_10 | 0.0122 | 0.019 |

Top 10 CEFX-II clones in Donor 5

| | | |
|---|---|---|
| CEFX-II_1 | 0 | 0.06 |
| CEFX-II_2 | 0 | 0.05 |
| CEFX-II_3 | 0 | 0.046 |
| CEFX-II_4 | 0 | 0.042 |
| CEFX-II_5 | 0 | 0.033 |
| CEFX-II_6 | 0 | 0.027 |
| CEFX-II_7 | 0.012 | 0.019 |
| CEFX-II_8 | 0 | 0.019 |
| CEFX-II_9 | 0 | 0.019 |
| CEFX-II_10 | 0 | 0.018 |

Statistical analysis of the most expanded APOB-AIM⁺ clone (Rank 1 in donor 6) using Fisher's exact test confirmed significant APOB₆-driven selection of this clone (frequencies in AIM⁻ and AIM⁺ templates) and shows the existence of an already expanded APOB₆-specific memory T cell pool in vivo (frequencies in naïve vs TCM or naïve vs TEM templates) (FIG. 4G). The nucleotide sequence of this TCR rearrangement and its bio-identity (translated CDR3 amino acid sequence and annotated V and J genes) are shown (FIG. 4H).

APOB₆-reactive CD4⁺ T cells are enriched in antigen-experienced memory markers.

Lineage-tracing of APOB-specific AIM⁺ TCR sequences in FIGS. 4A to 4H highlighted the existence of APOB₆-responsive memory CD4⁺ T cells in normal donors with no known conditions of heart disease. The inventors examined this further in an ex vivo (does not involve expansion) AIM assay of APOB₆-stimulated PBMCs from 20 new donors enrolled in a validation cohort of HLA-typed healthy participants (demographic details in Table 11, donor HLA-II alleles in Table 11).

TABLE 11
Demographic details of healthy donors in the validation cohort

| Donor | | | | Age (in |
|---|---|---|---|---|
| ID | Ethnicity | Race | Gender | years) |
| 3865 | Hispanic/Latino | Unknown | F | 27 |
| 3922 | Not Hispanic/Latino | Asian | F | 35 |
| 3992 | Not Hispanic/Latino | White | F | 66 |
| 4010 | Hispanic/Latino | White | M | 38 |
| 4022 | Hispanic/Latino | White | F | 62 |
| 4101 | Hispanic/Latino | Unknown | F | 25 |
| 4108 | Not Hispanic/Latino | White | M | 29 |
| 4109 | Hispanic/Latino | White | M | 52 |
| 4111 | Not Hispanic/Latino | White | M | 37 |
| 4121 | Not Hispanic/Latino | White | F | 38 |
| 4142 | Not Hispanic/Latino | White | M | 25 |
| 4148 | Not Hispanic/Latino | White | M | 61 |
| 4158 | Not Hispanic/Latino | Asian | M | 21 |
| 4161 | Not Hispanic/Latino | More | M | 27 |
| 4164 | Not Hispanic/Latino | White | F | 23 |
| 4166 | Hispanic/Latino | Unknown | M | 28 |
| 4173 | Unknown | White | F | 58 |
| 4181 | Hispanic/Latino | More | M | 20 |
| 4186 | Not Hispanic/Latino | Asian | F | 20 |
| 4187 | Not Hispanic/Latino | More | F | 20 |

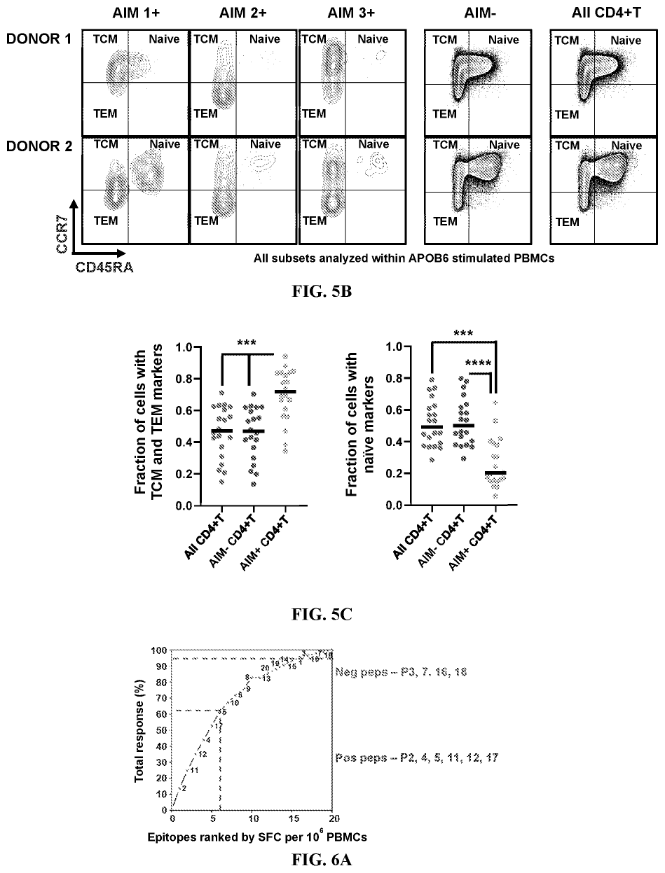

The short stimulation regime (24 h) avoids any potential non-specific skewing of phenotypes and therefore is most likely to reflect in vivo states. As before, responding and non-responding populations in peptide-stimulated PBMCs were defined using a sequential gating scheme (gating strategy in FIG. 16A) to evaluate expressions of common T cell activation markers CD40L, CD69, CD25, OX-40 and 4-1BB. Analysis of multiple activation marker combinations helped the inventors to capture all responding AIM⁺CD4⁺ T cells, irrespective of donor-to-donor heterogeneity in APOB₆-induced AIM marker expressions (representative plots in FIG. 5A). Comparison of naïve and memory (TCM+TEM) fractions within AIM⁻, AIM⁺ and all CD4⁺ T cells (representative plots in FIG. 5B) in APOB₆ Stimulated (FIG. 5C) and CEFX-11 stimulated (FIG. 16B) PBMCs, revealed significantly higher fractions of memory cells and lower fractions of naïve cells in AIM⁺CD4⁺ T cells, as compared to AIM⁻ or all CD4⁺ T cells. This confirmed that antigen-specific CD4⁺ T cells that respond to dominant APOB epitopes are enriched in antigen-experienced memory T cell features, corroborating the previous conclusions from the lineage-tracing experiment in FIG. 4.

APOB₆ triggers secretion of both proinflammatory and regulatory T helper cytokines.

A commercially available (BD Biosciences) human Cytometric Bead Array (CBA) kit was used to examine the full spectrum of T helper cytokines (IL-2, TNF, IFNγ, IL-17A, IL-4, IL-10) secreted in response to APOB₆ stimulation of PBMCs from donors in the validation cohort (Table 10). The inventors used a short (24 h) stimulation regime to avoid any phenotypic skewing that may occur in expansion-based methods. Ex vivo analysis also improved detection of IL-10 production, whose levels were very low in expansion-based ICS assays (FIGS. 9C,9D).

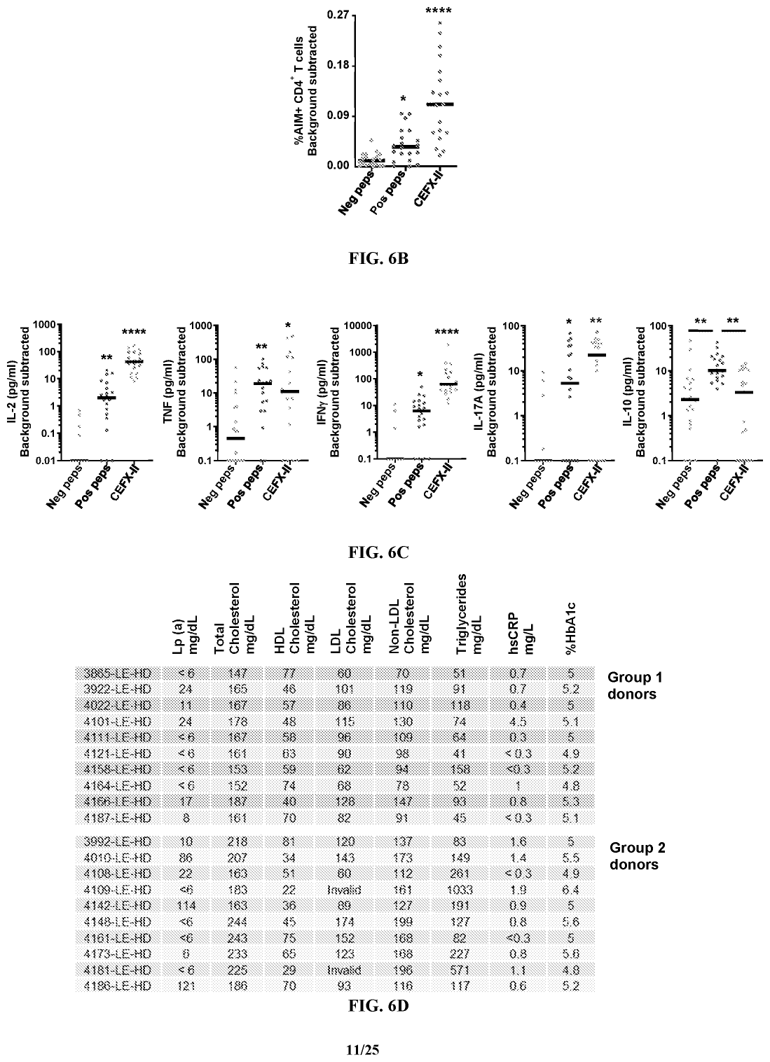

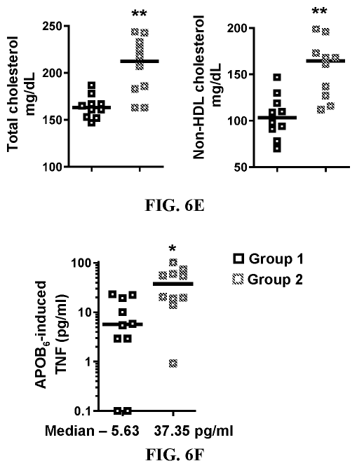

As negative control, a contrasting set of APOB peptides (“neg peps”) were selected that had exhibited lowest reactivity in the deconvolution experiment (FIGS. 3A to 3F). These peptides contributed <5% to the cumulative response (FIG. 6A) and had responder frequencies ≤10% (FIG. 3B). As compared to the dominant APOB peptides (“pos peps”), responses to them were ˜8-fold lower in a cellular proliferation assay and ˜3.5-fold lower in the 24 h ex vivo AIM assay (FIG. 6B).

It was found that both APOB₆ and CEFX-II pools induced significantly higher secretion of IL-2, TNF, IFNγ and IL-17A, as compared to the contrasting set of APOB peptides (FIG. 6C). Unlike these cytokines, significantly higher induction of the regulatory cytokine IL-10, was triggered only by the dominant self-antigen-derived APOB peptides, but not by CEFX-II pool of bacterial and viral epitopes (FIG. 6C). IL-4 was not detectable in the ex vivo assay.

APOB₆ induces stronger proinflammatory responses in normal donors with abnormal or borderline risk levels of CVD-related lipids and lipoproteins in the blood.

For all the donors in the validation cohort, the inventors had conducted lab tests (at UCSD Center for Advanced Laboratory Medicine (CALM)) for multiple clinical parameters (Lipoprotein(a), lipid panel, comprehensive metabolic panel, hsCRP, HbA1c) on the day of sample collection. The donors were segregated into two groups based on their lipid profile and Lp(a) levels. While none of the donors had any known conditions of heart disease, donors in group 2 exhibited abnormal or borderline risk levels of either Lp(a) [>30 mg/dL] or total cholesterol [>200 mg/dL] or HDL cholesterol [<40 mg/dL] or triglycerides [>200 mg/dL](FIG. 6D). These Group 2 donors, who had significantly higher levels of total and APOB-containing non-HDL cholesterol (FIG. 6E), also secreted increased amounts of the proinflammatory cytokine TNF (Median 37.35 vs 5.63 pg/ml, Group 2 vs 1) upon ex vivo stimulation with APOB₆ pool (FIG. 6F). Levels of other APOB₆-induced T helper cytokines did not differ significantly between the two groups.

These data collectively show that the dominant epitopes in APOB represent suitable candidates to monitor atherosclerosis-related autoreactive proinflammatory and regulatory responses in the general population.

CD4⁺ T cell activation, memory marker expression and proinflammatory cytokine responses to APOB₆ are heightened in CAD patients with higher disease burden.

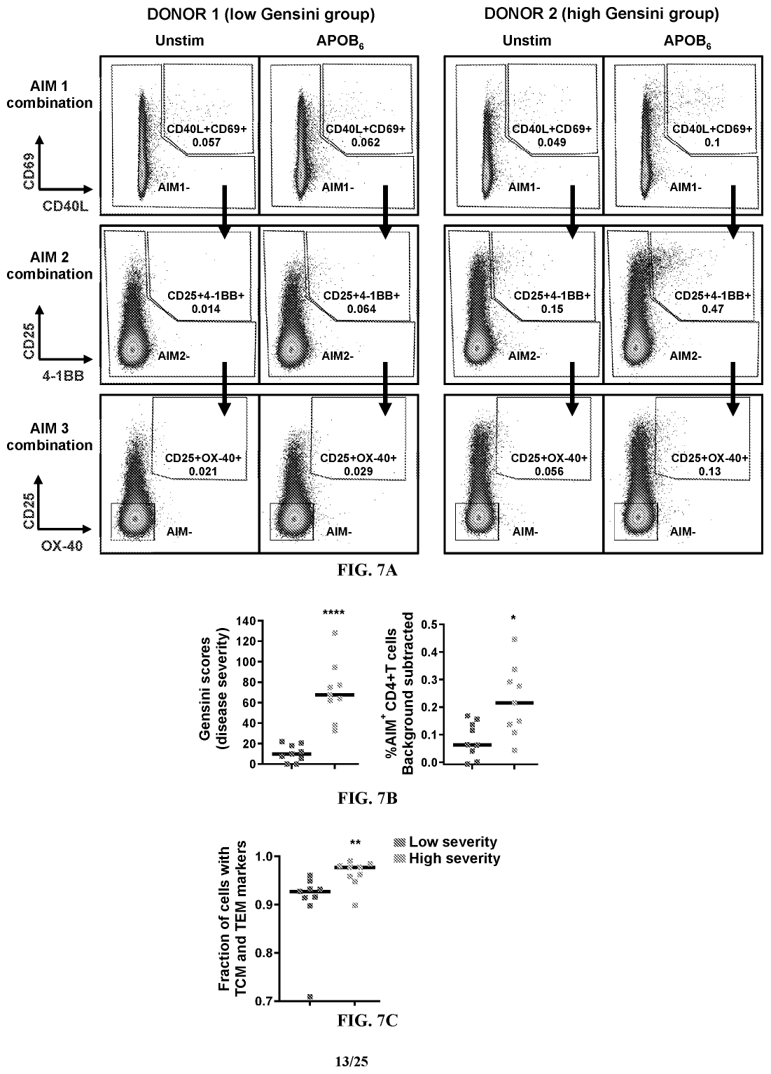

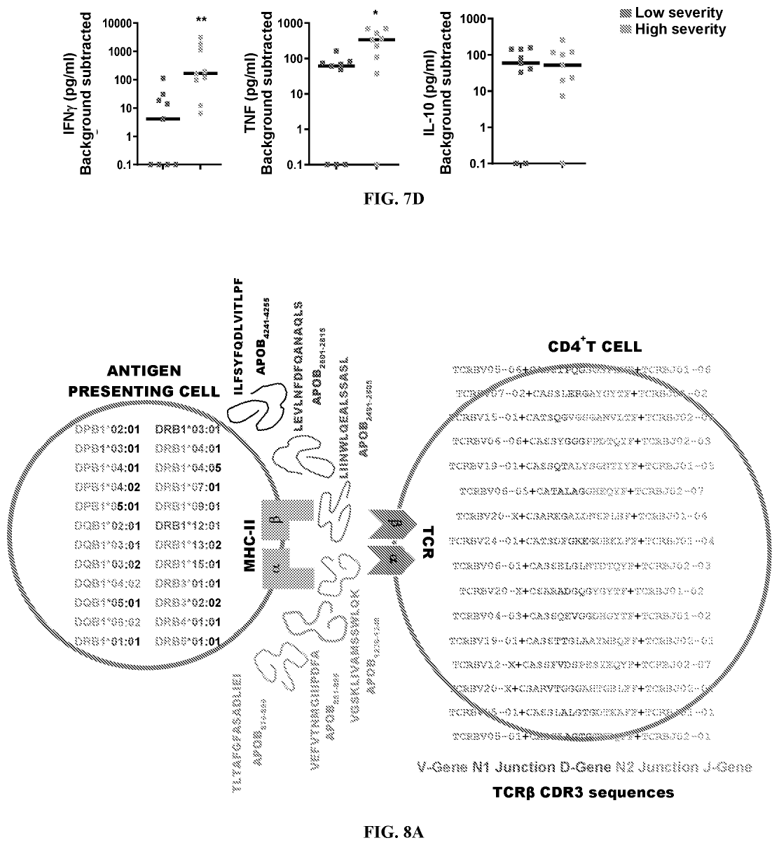

To examine APOB₆-specific responses in patients with coronary artery disease (CAD), clinical samples were analyzed from the Coronary Assessment in Virginia (CAVA) cohort. CD4⁺ T responses to APOB₆ were compared in matched clinical samples from patients with high and low CAD, as assessed by angiographic disease burden, expressed as Gensini scores⁴⁸. In the ex vivo (24 h) AIM assay, stimulation of PBMCs with APOB₆ pool triggered stronger CD4⁺ T cell activation and higher expression of multiple AIM marker combinations (FIG. 7A) in a patient with more severe CAD (Gensini 75), as compared to a patient with less severe disease (Gensini 0). Evaluation of APOB₆-induced expressions of all AIM combinations in 18 matched CAD patients, detected ˜3.5-fold higher frequencies of AIM⁺CD4⁺ T cells in patients with higher Gensini scores (median % AIM⁺CD4⁺ T 0.06 vs 0.21; FIG. 7B).

APOB-specific AIM⁺CD4⁺ T cells in this high severity disease group exhibited greater skewing towards antigen-experienced phenotypes and contained significantly higher fraction of memory T cells (CD45RA⁻CCR7⁺ central memory and CD45RA⁻CCR7⁻ effector memory) (FIG. 7C), than the AIM⁺CD4⁺ T cells in the low severity group.

Next, cytokine responses to APOB₆ were monitored in these patient samples. The inventors detected significantly higher levels of secreted proinflammatory cytokines TNF (˜5.5-fold; median 61.2 vs 335.3 pg/ml) and IFNγ (˜40-fold; median 4.1 vs 164.6 pg/ml) in patients with more severe CAD than in the less severe group (FIG. 7D). In contrast, APOB₆-induced IL-10 levels were similar in both groups (median 59 vs 52 μg/ml, FIG. 7D). Taken together, these data demonstrate that autoimmune CD4⁺ T activation in response to dominant APOB epitopes and the resulting proinflammatory cytokines TNF and IFN-γ are highly informative, antigen-specific immune readouts of disease severity in CAD patients.

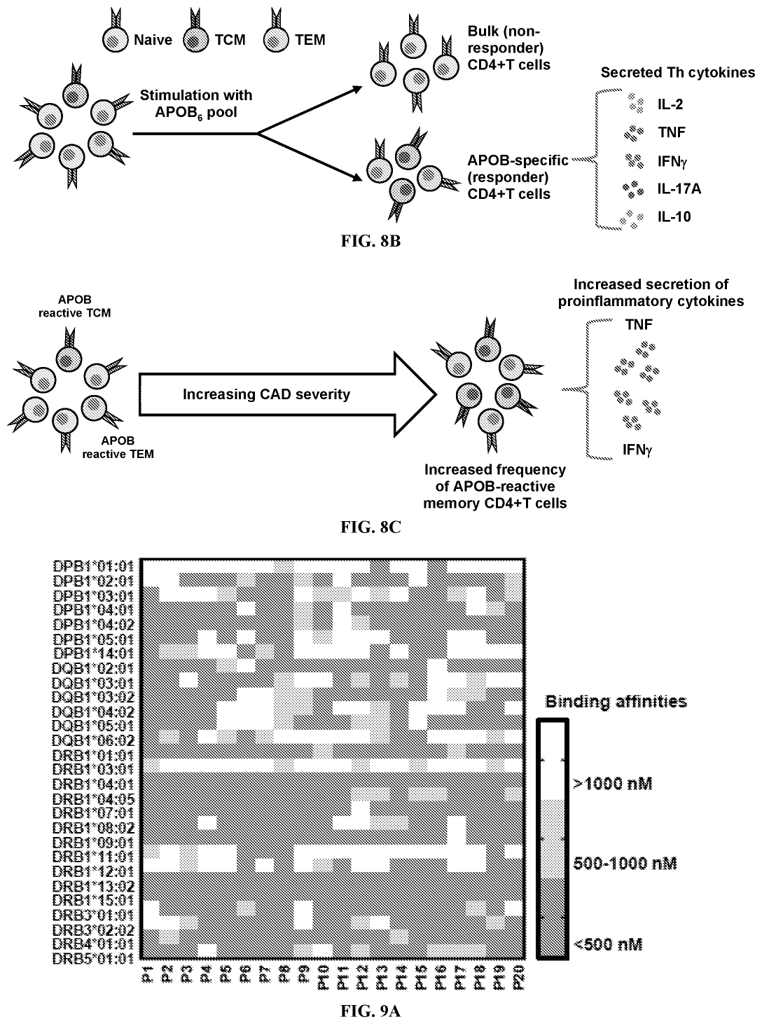

In summary, the inventors systematically examined the antigenicity of an atherosclerosis-related autoantigen, the human APOB protein, and delineated the identities of all components of an immunodominant anti-APOB CD4⁺ T trimolecular complex—the epitopes, epitope binding human HLA-II alleles and human autoreactive TCR clones that are activated by the APOB epitopes (FIG. 8A, SEQ ID NOS:179, 224, 225, 180, 181, 183, 184, 214, 342, 190, 191, 193, 194, 195). Stimulation with a combined pool of these six immunodominant peptides detected APOB-responsive memory CD4⁺ T cells and triggered secretion of both proinflammatory and regulatory cytokines (FIG. 8B) in normal donors with diverse HLA-II allelic background. In clinical samples, APOB₆ stimulation detected heightened frequencies of activated CD4⁺ T cells, increased skewing towards memory phenotype and augmented secretion of proinflammatory cytokines TNF and IFNγ in patients with more severe disease (FIG. 8C).

Recent research has established a critical role of the immune system in shaping atherosclerosis³. Adaptive immune responses, defined by strict antigen-receptor specificity, represent precise targets for monitoring and understanding disease-specific immune responses. Recent single cell sequencing studies revealed the presence of T cells expressing activation and memory markers and oligoclonal populations of proliferated T cells in human plaques^{10,11}. However, the identities of the antigenic proteins and putative epitopes that trigger such atherosclerosis-related human T cell activation and proliferation remain unknown. Preclinical studies and clinical correlations of autoantibodies against apolipoprotein B, the LDL core protein, suggest its relevance as a major atherosclerosis-related antigen^{12,13} Two recent studies from the present inventors demonstrated the presence of APOB-specific CD4⁺ T cells in human blood^{1,2} suggesting that human autoreactive T cells respond to APOB. However, immunodominant HLA-II-restricted CD4⁺ T cell epitopes in APOB have not been reported.

The inventors developed a restimulation-based workflow that allowed sensitive detection of statistically significant APOB-specific CD4⁺ T cell responses in the general population. The presence of epitope-specific autoreactive T cells in blood of healthy people, sometimes at frequencies similar to those in diseased individuals, are also reported in other autoimmune-related conditions^{5,6,24,49}. Six immunodominant APOB epitopes were mapped in HLA-typed donors and delineated TCRs of the autoreactive CD4⁺ T clones using high-throughput sequencing strategies. APOB-specific responding T cells were enriched in memory phenotypes. In comparison to a contrasting set of poorly immunogenic APOB peptides, the dominant epitopes (APOB₆) triggered stronger CD4⁺ T activation and robust secretion of both pro-inflammatory and regulatory T helper cytokines. Finally, APOB₆-dependent CD4⁺ T activation was monitored, charted memory marker expression in the responding cells and profiled secreted T helper cytokines in donor PBMCs from matched CAD patients with high and low disease severity. The integrated workflow described here represents an optimized strategy to interrogate the dynamic behavior (fluctuations in frequencies and phenotypes, expansion and proliferation of specific public or private TCRs) of autoreactive T cells under homeostatic and diseased conditions.

Immuno-profiling of CDR3 sequences have been employed to track disease-specific autoreactive T cell clones in longitudinal samples, case-control studies and across different immune compartments in various autoimmune conditions⁵⁰⁻⁵². Unique disease-related clones that can discriminate between two autoimmune disorders have been identified through TCRβ repertoire analysis of peripheral blood samples from patients of systemic lupus erythematosus or rheumatoid arthritis and from healthy controls⁵³. Preexisting T cells undergo recall responses upon sensitization with relevant autoantigens and exhibit disease-associated fluctuations in frequencies, memory phenotypes, cytokine secretion and tissue distribution^{52,54}. Identification of their TCR sequences has improved the understanding of epitope-specific autoreactive responses⁵⁵ and has opened up new avenues for improved disease prognosis^{53,56} and targeted intervention⁵⁷.

These results demonstrate the constitutive CD4⁺ T cell response to APOB in humans. For the first time, the molecular triad is resolved at the sequence level: human MHC-II, human TCRβ CDR3 sequences, and six immunodominant human APOB peptides that engage these MHC and TCR molecules. Proinflammatory antigen-experienced autoimmune responses to these dominant APOB epitopes escalate in clinical atherosclerosis.

Study subjects. Two different cohorts of healthy subjects were recruited through La Jolla Institute's Clinical core. First, a screening cohort, which included participants enrolled in the Normal Blood Donation Program (NBDP donor age and gender details in Table 3). The donors were pre-screened to confirm the absence of any significant systemic disease or viral infections including hepatitis B or C, and HIV. Ethical approval for the study was provided by the Institutional Review Boards at La Jolla Institute for Immunology (VD-057). For the second validation cohort of healthy adult donors (demographics in Table 10), donor blood samples were also sent to clinical labs at UCSD Center for Advanced Laboratory Medicine (CALM) for evaluating Lp(a), lipids, metabolites, hsCRP and HbA1c, on the day of sample collection. None of the donors had COVID 19 (was an exclusion criteria) or any other ongoing infection. They had no known conditions of cancer, diabetes, heart or kidney or liver disease. Donors were neither pregnant nor nursing. The study was approved by the Institutional Review Boards at La Jolla Institute for Immunology (IB-248-0821) and at University of California, San Diego (IRB 190053). Written informed consents were obtained from all participants enrolled in either cohort.

For the clinical Coronary Assessment in Virginia (CAVA) cohort, we obtained cryopreserved peripheral blood mononuclear cells from 18 patients (Table 8) undergoing standard cardiac catheterization at the Cardiac Catheterization laboratory at the University of Virginia Health System, Charlottesville, Virginia, USA. Written informed consents were obtained from all participants before enrollment. Blood samples were collected prior to cardiac catheterization. The study was approved by the Human Institutional Review Board (IRB No. 15328) at the University of Virginia. In this study, Quantitative Coronary Angiography (QCA) was performed using automatic edge detection from an end-diastolic frame, which was selected for each lesion, based on demonstration of the most severe stenosis with minimal foreshortening and branch overlap. The minimum lumen diameter, reference diameter, percent diameter stenosis, and stenosis length were calculated by blinded, experienced investigators who assessed disease severity based on the Gensini score¹. Briefly, each artery segment was assigned a score of 0-32 based on the percent stenosis. For each segment, this score was multiplied by 0.5-5, depending on the location of the stenosis. Scores for all segments are then added together to given a final score of angiographic disease burden. Score adjustment for collateral was not performed for this study.

HLA binding predictions and experimental validation. 911 peptides (15-mers that overlap by <10 residues) spanning the entire human APOB protein (UniProtKB P04114) were scored for their ability to bind a reference set of 27 frequent and representative HLA DP, DQ and DR alleles² using MHC-II binding prediction tools available in the Immune Epitope Database (www.iedb.org). For each peptide, allele-specific consensus median percentile ranks were generated from all algorithms queried by the IEDB tool. A peptide with a median percentile score <20 was considered a binder to a specific allele², and for each peptide the number of alleles predicted to be bound was tabulated. A set of 30 peptides predicted to bind ≥20 alleles was selected as initial candidates.

Next, the capacity of these peptides to bind 28 common HLA alleles (27 from the reference set plus DPB1*03:01) was determined experimentally using classical competition assays based on the inhibition of binding of high affinity radiolabeled ligands to purified MHC molecules³. Under the conditions utilized, where [radiolabeled peptide] is <[MHC], and [MHC]<[inhibitor peptide], the measured IC50 values, defined as the concentrations of inhibitor peptide that inhibits the binding of the radiolabeled ligand by 50%, are reasonable estimates of true K_{d} values. Binding to a specific allele was defined as IC50<1000 nM.

Peptides. Human APOB-derived peptides were synthesized as crude material (>70% purity) on a 5 mg scale by TC Peptide Lab (San Diego). Each peptide was resuspended in dimethyl sulfoxide (DMSO, Sigma Aldrich) and combined in equal proportions to prepare the APOB₂₀ peptide pool. A subset of the peptides was synthesized as purified material (>95% purity by reversed-phase HPLC, confirmed by mass spectrometry) on a 2 mg scale by TC Peptide Lab (San Diego). CEFX-II (PM-CEFX-3), a positive control pool of 68 known MHC class-II restricted peptides from different infectious agents, the negative control pool of 92 15-mer peptides spanning the human actin alpha protein (PM-ACTS), and custom-made APOB peptides (P2, P4, P5, P11, P12, P17) were purchased from JPT Peptide Technologies (Berlin, Germany).

Scrambled versions of APOB epitopes were generated using the tool at www.bioinformatics.org/sms2/shuffle_protein.html. Both original and scrambled peptides were synthesized by TC Peptide Lab (San Diego).

HLA Typing. Genomic DNA was isolated from donor PBMCs using REPLI-g DNA midi kit (QIAGEN). HLA typing with Illumina next generation sequencing was done using services provided by an ASHI-accredited laboratory at the Institute for Immunology & Infectious Diseases, Murdoch University, Western Australia). Class I genes—HLA A, B, C and Class II genes DPB1, DQA1, DQB1, DRB1, DRB3, DRB4 and DRB5, were resolved using exon specific targeted PCR amplification of genomic DNA. Filtered reads were passed through a proprietary algorithm, IIID Allele caller, and mapped using the ASHI-accredited IIID HLA Analysis suite and the latest human HLA allele reference sequences from ImMunoGeneTics (IMGT) HLA database.

Isolation of Peripheral blood mononuclear cells (PBMCs) and cell culture. Venous blood samples were collected in blood bags or tubes coated with anti-coagulant (sodium heparin or K2-EDTA). For clinical tests, samples were collected either in Lithium heparin tubes (for comprehensive metabolic panel, lipid panel, Lp(a), hsCRP) or K2-EDTA tubes (for HbA1c). PBMCs were isolated from whole blood by Ficoll-Paque (Sigma Aldrich) density-gradient centrifugation. For cryopreservation in liquid nitrogen, cells were re-suspended in CryoStor® CS10 (Stemcell), a serum-free and animal component-free cryopreservation medium containing 10% DMSO. All in vitro cultures and re-stimulation regimes were carried out in serum-free cell culture medium (TexMACS™, Miltenyi Biotech) supplemented with 1% penicillin/streptomycin (Thermo Fisher Scientific) and cultured at 37° C. with 5% CO₂.

Expansion of antigen-specific T cells and Enzyme-linked immunospot (ELISpot) assay. For in vitro expansion, PBMCs were plated at a density of 2×10⁶ cells/ml in 24-well plates and were cultured in the presence of APOB₂₀ and CEFX-II peptide pools. 10 U/ml human IL-2 (Invitrogen) was added to the media at Days 4, 7 and 10. On Day 14, cells were washed and re-plated in 96-well ELISpot plates (Millipore) coated with mouse anti-human IFNγ (clone 1-D1K) antibody (Mabtech). PBMCs were re-stimulated with cognate or irrelevant peptide pools (for crossover stimulation experiments) or with individual APOB-derived peptides (for deconvolution and epitope mapping). Unstimulated and PHA-L stimulated sets served as negative and positive controls, respectively. APOB₂₀ (20 peptides) and CEFX-II (68 peptides) pools were used at peptide concentrations 5 μg/ml and 1.5 μg/ml, respectively. Individual APOB peptides were used at 20 μg/ml final concentrations. APOB₆ pool of six dominant epitopes was used at 10 μg/ml concentration. Positive control Phytohemagglutinin-L (PHA-L, eBioscience) was used at 1× concentration. After 24 h incubation, secreted IFNγ cytokine was detected using mouse anti-human IFNγ (clone 7-B6-1) biotinylated antibody (Mabtech). Wells were imaged and spot-forming cells (SFCs) were quantified using the Zeiss KS or AID (Autoimmun Diagnostika) ELISpot readers.

In experiments involving HLA blockade, HLA-DR (clone L243), DP (clone B7/21), DQ (clone SVPL3) or pan HLA class I (W6/32) monoclonal antibodies (kindly provided by Dr. Alessandro Sette, LJI) were added at 10 μg/ml concentration, 30 min prior to restimulation with peptides.

In vitro expansion and intracellular cytokine staining (ICS) assay. PBMCs were plated at a density of 2×10⁶ cells/ml in 24-well plates and were cultured with desired peptides or peptide pools. 10 U/ml IL-2 was added at Days 4, 7 and 10. After 14 days of in vitro expansion in the presence of individual epitopes or peptide pools, PBMCs were harvested, washed and re-plated in U-bottom 96-well plates. PBMCs at 1×10⁶ cells per conditions were re-stimulated with desired sets of cognate or irrelevant pools and peptides. APOB₂₀ (20 peptides) and CEFX-II (68 peptides) pools were used at peptide concentrations 5 μg/ml and 1.5 μg/ml, respectively. Individual APOB peptides were used at 20 μg/ml. After 2 h, protein transport inhibitor cocktail (eBioscience) was added at 1× concentration and incubated for an additional period of 4 h. After the 6 h stimulation period, cells were washed with FACS buffer (PBS w/o Ca/Mg, 2% FCS) and resuspended in staining master mix containing anti-human Fc-Block (Biolegend), fixable viability dye and antibodies against T cell and non-T cell (Dump) surface markers (antibody details in Table 4). Viability dye at 1:1000 dilution and antibodies at 1:200 dilutions were used. For intracellular cytokine detection, cells were fixed, permeabilized and stained in standard buffer solutions (eBioscience). Cells were stained for CD40L and T helper cytokines (antibody details in Table 4). Antibodies against intracellular markers were used at a final dilution of 1:50. Data was acquired on a BD LSR II flow cytometer and analyzed with FlowJo software.

Activation-induced marker (AIM) assay. In ex vivo AIM assay, 1×10⁶ PBMCs per condition were plated in flat-bottomed 96-well plates and cultured for 24 h in the presence of the indicated peptide pools. To account for differences in the number of peptides in each pool, peptides in the APOB₂₀ pool (20 peptides) were used at 10 μg/ml, CEFX-II pool (68 peptides) at 3 μg/ml and Actin pool (92 peptides) at 2 μg/ml, so that total concentration of the pools remain comparable. APOB₆ pool of dominant epitopes (“pos peps”) and the pool of poorly immunogenic APOB peptides (“neg peps”) were each used at 20 μg/ml per peptide. To improve surface detection of CD40L, a CD40 blocking antibody (Novus Biologicals) was added to each well 15 min before stimulation at a final concentration of 1 μg/ml. In experiments with HLA-II blocking, purified mouse Anti-human HLA-DR, DP, DQ (clone Tu39, BD Biosciences) was added to each well 30 min before stimulation at a final concentration of 20 μg/ml. At the end of the stimulation period, cells were washed with FACS buffer (PBS w/o Ca/Mg, 2% FCS) and resuspended in a staining master mix containing anti-human Fc-Block (Biolegend), fixable viability dye and antibodies against T cell and non-T cell (Dump) surface markers, activation markers and memory markers (antibody details in Table IV). Viability dye was used at 1:1000 dilution, antibodies were used at a final dilution of 1:100 (activation markers) and 1:200 (other extracellular markers). Data was acquired on a BD LSR II flow cytometer and analyzed with FlowJo software.

RATE analysis. The RATE (Restrictor Analysis Tool for Epitopes) tool (iedb-rate.liai.org/) was used to computationally infer HLA restriction of the twenty APOB peptides based on observed responses (FIG. 3) in HLA typed donors^{4,5}. RATE creates contingency tables between donor alleles and responder frequencies and estimates Relative Frequency (RF) and Odds ratio to determine associations between the expression of a specific allele in a donor and the occurrence of a positive immune response in that donor. An RF >1 indicates a positive association between an allele and a response. RF is calculated according to the formula:

$$R F = [ {A}^{+} {R}^{+} / ( {A}^{+} {R}^{+} + {A}^{+} {R}^{-} ) ] / ( {A}^{+} {R}^{+} + {A}^{-} {R}^{+} ) / Total Donors ]$$

where
A⁺R⁺=Number of subjects who expressed a specific allele and gave a positive immune response to the specific peptide
A⁻R⁻=Number of subjects who did not express the specific allele and did not give a positive immune response to the specific peptide
A⁻R⁺=Number of subjects who did not express the specific allele but gave a positive immune response to the specific peptide
A⁺R⁻=Number of subjects who expressed the specific allele but did not give a positive immune response to the specific peptide

Statistical significance of an association is determined using the Fisher's exact test.

Single HLA-II transfected cell lines. RM3 (derived from human B lymphocyte cell line Raji) cells transfected with either DPB1*05:01 or DRB3*02:02 HLA-II alleles⁶ (kindly provided by Dr. Alessandro Sette, LJI) were used to examine the immunogenicity of APOB epitopes in the context of defined peptide-HLA-II binder, non-binder scenarios. In preparation for the assay, donor PBMCs expressing the HLA-II allele under study were expanded with the binder or the non-binder APOB peptide at 20 μg/ml concentration (intermittent 10 U/ml IL-2 feeding at days 4, 7 and 10). The single HLA-II transfected cell lines were maintained in culture. At day 14 of the PBMC expansion protocol, each transfected cell line was plated at 2×10⁵ cells/well in a flat-bottom 96-well plate and pulsed with 20 μg/ml individual peptide for 1 h at 37° C. Cells without any added peptide served as “no pep” background control. Cells were washed four times in PBS to remove free unbound peptides. Peptide-expanded PBMCs, at 2×10⁵/well, were plated in 96-well ELISpot plates (Millipore) coated with mouse anti-human IFNγ (clone 1-D1K) antibody (Mabtech). Peptide-pulsed and control “no pep” cell lines were added at 5×10⁴ cells/well. No exogenous peptides were added to the wells containing PBMCs and cell lines. Separate unstimulated and cognate peptide-stimulated (20 μg/ml) PBMC alone sets served as negative and positive controls, respectively. After 24 h incubation, secreted IFNγ cytokine was detected using mouse anti-human IFNγ (clone 7-B6-1) biotinylated antibody (Mabtech). Wells were imaged using the AID (Autoimmun Diagnostika) ELISpot reader.

Cell isolation and T cell receptor sequencing. For isolation of APOB epitope-specific CD4⁺ T cells, PBMCs were expanded with the APOB₆pool (10 μg/ml per peptide). On Day 14, cultured cells were washed and re-stimulated with APOB₆ (10 μg/ml) for 24 h (AIM assay protocol). 1 μg/ml of CD40 blocking antibody was added 15 min before stimulation. At the end of the stimulation period, cells were washed with FACS buffer (PBS w/o Ca/Mg, 2% FCS). Day 14 (APOB₆ expanded and restimulated) and Day 0 (ex vivo samples with no in vitro stimulation) PBMC samples from six donors were resuspended in a staining master mix containing anti-human Fc-Block (Biolegend), fixable viability dye and antibodies against T cell and non-T cell (Dump) surface markers, activation markers and memory markers (antibody details in Table 4). Viability dye was used at 1:1000 dilution, antibodies were used at a final dilution of 1:100 (activation markers) and 1:200 (other extracellular markers). AIM⁺CD4⁺ T cells were identified in a serial gating scheme where expressions of four combinations of CD40L, CD69, CD25, 4-1BB and OX-40 activation markers were sequentially assessed. Gates were set based on unstimulated and PHA-L stimulated negative and positive controls, respectively. Antigen-specific AIM⁺ and non-specific control AIM⁻ CD4⁺ T cells from Day 14 samples and naïve (CD45RA⁺CCR7⁺), central memory (CD45RA⁻CCR7⁺, TCM) and effector memory (CD45RA⁻CCR7⁻, TEM) CD4⁺ T cells from Day 0 PBMCs were sorted into HEPES-containing buffer (PBS with 2% FBS and 0.025 M HEPES) using BD FACSAria flow cytometry sorters. Acquired data was analyzed with FlowJo software.

Genomic DNA was extracted from sorted cells using QIAamp DNA Micro Kit (QIAGEN) and sent to Adaptive Biotechnologies for survey-level TCRβ sequencing (depth ˜60,000 reads) using their established immunoSEQ platform⁷. Briefly, highly variable CDR3 regions on somatically rearranged human TCRβ chains were amplified with a two-step multiplex PCR approach using optimized set of primers that target the VDJ region spanning each unique CDR3B. A synthetic repertoire containing all possible V/J templates is used as a built-in control to quantify and correct PCR amplification biases. Processing of raw Illumina sequence reads, filtering, demultiplexing, clustering and mapping of CDR3 sequences and annotation of VDJ genes using IMGT database sequences were performed at Adaptive Biotechnologies and made available for download and analysis. Details of input DNA and total (productive templates) and unique (productive rearrangements) TCR counts for Day 0 (Naïve, TCM, TEM) and Day14 APOB-AIM⁺ and AIM⁻ CD4⁺ T subsets in all donors are provided in Table 7. TCR repertoire characteristics and individual rearrangements were analyzed using immunoSEQ Analyzer 3.0.

Proliferation assay. 10 million PBMCs were resuspended in 1 ml PBS buffer. Cells were labelled with 5-chloromethylfluorescein diacetate (CellTrace™ CFSE Cell Proliferation Kit, Invitrogen) at a working concentration of 1 M. Cells were thoroughly mixed with CFSE and incubated at 37° C. for 15 min, with intermittent mixing. Cells were washed twice with PBS containing 20% FBS. Labelled cells were plated in a 48-well plate with 1 million cells per well. PBMCs were stimulated with either the APOB₆ pool of dominant epitopes (“pos peps”) or with the pool of poorly immunogenic APOB peptides (“neg peps”), both used at 10 μg/ml per peptide. Unstimulated sets were used as negative controls to subtract background. A set of PBMCs stimulated with soluble CD3/CD28 (ImmunoCult™ Human CD3/CD28 T Cell Activator) was used as a positive control. After 5 days, media was replenished with fresh culture medium. On day 10, cells were harvested, washed and stained with viability dye, non-T cell markers (Dump) and T cell markers (antibody details in Table 4). Viability dye at 1:1000 dilution and antibodies at 1:200 dilution was used. Data was acquired on a BD LSR II flow cytometer and analyzed with FlowJo software. “No CFSE” set was used as control in flow cytometry.

Cytometric Bead Array. 2×10⁶ PBMCs per condition were plated in flat-bottomed 96-well plates and stimulated with “pos pep” or “neg pep” APOB peptides (20 μg/ml) or with CEFX-II (3 μg/ml per peptide). After 24 h of incubation, plates were centrifuged at 500×g for 5 min at 4° C. Supernatants were collected and frozen at −80° C. until further analysis with Human Th1/Th2/Th17 cytokine kit (BD Biosciences, Catalog No. 560484), performed according to manufacturer's instructions.

Statistical analysis. Data analysis and statistical comparisons were done using GraphPad Prism. All statistical tests, significance of p values and axes descriptions are detailed in the legends of respective figures.

TABLE 12
List of Sequences.

| SEQ | |
|---|---|
| ID | |
| NO: | Sequence |
| 1 | AACGCCTTGTTGCTGGGGGACTCGGCCCTCTATCTCTGTGCCAGCAGCATCCCACAGGGTTCT |
| | GGTTCACCCCTCCACTTTGGGAAT |
| 2 | GAGTTGGCTGCTCCCTCCCAGACATCTGTGTACTTCTGTGCCAGCAGTTACGGGGGAGGGCCG |
| | CCAGATACGCAGTATTTTGGCCCA |
| 3 | TCGGCCCAAAAGAACCCGACAGCTTTCTATCTCTGTGCCAGTAGTCAAACCGCTCTTTACTCT |
| | GGAAACACCATATATTTTGGAGAG |
| 4 | NNNNNNNTGTCGGCTGCTCCCTCCCAGACATCTGTGTACTTCTGTGCCACCGCCTTAGCGGGA |
| | GGACACGAGCAGTACTTCGGGCCG |
| 5 | ACCAGTGCCCATCCTGAAGACAGCAGCTTCTACATCTGCAGTGCTAGAGAAGGGGCACTTGAT |
| | AATTCACCCCTCCACTTTGGGAAT |
| 6 | TCTGCCATCCCCAACCAGACAGCTCTTTACTTCTGTGCCACCAGTGATTTCGGGAAAGAAGGG |
| | GATGAAAAACTGTTTTTTGGCAGT |
| 7 | TTGGAGATCCAGCGCACAGAGCAGGGGGACTCGGCCATGTATCTCTGTGCCAGCAGCTTTACT |
| | TATGATGAGCAGTTCTTCGGGCCA |
| 8 | CAGCGCACAGAGCAGGGGGACTCGGCCATGTATCTCTGTGCCAGCAGCCCCGGAAGGGAACCT |
| | GGAAACACCATATATTTTGGAGAG |
| 9 | AAGATCCAGCCCTCAGAACCCAGGGACTCAGCTGTGTACTTCTGTGCCAGCACGGCCACGGAG |
| | AGCTCACCCCTCCACTTTGGGAAT |
| 10 | CTGGAGTCGCCCAGCCCCAACCAGACCTCTCTGTACTTCTGTGCCAGCCGAAAAACGGGAAGT |
| | TTATATGGCTACACCTTCGGTTCG |
| 11 | AGCACCTTGGAGCTGGGGGACTCGGCCCTTTATCTTTGCGCCAGCAGCTTAAGGACAGGGATA |
| | ATCAATGAGCAGTTCTTCGGGCCA |
| 12 | TCGGCCCAAAAGAACCCGACAGCTTTCTATCTCTGTGCCAGTAGTACCACGGGACTAGCGGCA |
| | TACAATGAGCAGTTCTTCGGGCCA |
| 13 | CCCTCAGAACCCAGGGACTCAGCTGTGTACTTCTGTGCCAGCAGTTTTGTGGACAGCCCCGAA |
| | TCCTACGAGCAGTACTTCGGGCCG |
| 14 | ATTCTGGAGTCCGCCAGCACCAACCAGACATCTATGTACCTCTGTGCCAGCAGTTTAACCCGG |
| | ATTCCCGAGCAGTACTTCGGGCCG |
| 15 | GCCCATCCTGAAGACAGCAGCTTCTACATCTGCAGTGCTAGAGTAACAGGCGGGGGGGCCCAC |
| | ACCGGGGAGCTGTTTTTTGGAGAA |
| 16 | ACCTTGGAGCTGGGGGACTCGGCCCTTTATCTTTGCGCCAGCAGCTTGGCGCTTGGGACAGGG |
| | GACACTGAAGCTTTCTTTGGACAA |
| 17 | AGCACCTTGGAGCTGGGGGACTCGGCCCTTTATCTTTGCGCCAGCAGCTTGGCGGGGACAGGG |
| | ACAAATGAGCAGTTCTTCGGGCCA |
| 18 | GTGACATCGGCCCAAAAGAACCCGACAGCTTTCTATCTCTGTGCCAGTAGAGGGCAGAGGGAC |
| | ACCGGGGAGCTGTTTTTTGGAGAA |
| 19 | AACCTGAGCTCTCTGGAGCTGGGGGACTCAGCTTTGTATTTCTGTGCCAGCAGCATCCGACCC |
| | AACGATGAGCAGTTCTTCGGGCCA |
| 20 | NTGTCGGCTGCTCCCTCCCAGACATCTGTGTACTTCTGTGCCAGCAGTTACTCGAGCGGGGGA |
| | GGCAATGAGCAGTTCTTCGGGCCA |
| 21 | CCCAGCCCCAACCAGACCTCTCTGTACTTCTGTGCCAGCAGAACCATAGGACAGGGGTCCTTT |
| | AACTATGGCTACACCTTCGGTTCG |
| 22 | GAGTCCGCCAGCACCAACCAGACATCTATGTACCTCTGTGCCAGCAGTTCCTTAACCCCGGAG |
| | GCCTATGGCTACACCTTCGGTTCG |
| 23 | GTGACCAGTGCCCATCCTGAAGACAGCAGCTTCTACATCTGCAGTGCTATGTCCCCTAGCGGG |
| | ACCGGGGAGCTGTTTTTTGGAGAA |
| 24 | ATGAGCTCCTTGGAGCTGGGGGACTCAGCCCTGTACTTCTGTGCCAGCAGCGACGATCTGGCG |
| | GCCAACGTCCTGACTTTCGGGGCC |
| 25 | GCCTTGGAGCTGGAGGACTCGGCCCTGTATCTCTGTGCCAGCAGCCCTAGAGCAGGGGGCGCC |
| | GTGAGAGAGCAGTACTTCGGGCCG |
| 26 | AAGCTCCTTCTCAGTGACTCTGGCTTCTATCTCTGTGCCTGGAGTGTGGGAGGTCCTGGAGCC |
| | AATTCACCCCTCCACTTTGGGAAC |
| 27 | CTTCACATCAATTCCCTGGAGCTTGGTGACTCTGCTGTGTATTTCTGTGCCAGCAGCAAAGTT |
| | AATGAAAAACTGTTTTTTGGCAGT |
| 28 | AGCACCTTGGAGCTGGGGGACTCGGCCCTTTATCTTTGCGCCAGCAGCTTGGGAGGTCGGAGC |
| | AATCAGCCCCAGCATTTTGGTGAT |
| 29 | AGTGCCCATCCTGAAGACAGCAGCTTCTACATCTGCAGTGCTAGAGAACCCCATGACACGAGC |
| | ACAGATACGCAGTATTTTGGCCCA |
| 30 | GTGACCAGTGCCCATCCTGAAGACAGCAGCTTCTACATCTGCAGTGCTAGTCGTACGTATGAC |
| | AATCAGCCCCAGCATTTTGGTGAT |
| 31 | GCCCAAAAGAACCCGACAGCTTTCTATCTCTGTGCCAGTAGTTGGGACAGGACCAAGTTTGGC |
| | AATCAGCCCCAGCATTTTGGTGAT |
| 32 | AATTCCCTGGAGCTTGGTGACTCTGCTGTGTATTTCTGTGCCAGCAGCTCTAGCGGGAGTAAC |
| | TACAATGAGCAGTTCTTCGGGCCA |
| 33 | ACTCTGACAGTGACCAGTGCCCATCCTGAAGACAGCAGCTTCTACATCTGCAGTGCTAGATGG |
| | TACAATGAGCAGTTCTTCGGGCCA |
| 34 | CAGCGCACAGAGCAGGAGGACTCGGCCGTGTATCTCTGTGCCAGCAGCCCCCTCCTGTACTCT |
| | GGAAACACCATATATTTTGGAGAG |
| 35 | NTGTCGGCTGCTCCCTCCCAAACATCTGTGTACTTCTGTGCCAGCAGCCTACCCCGGGACAGG |
| | GACTATGGCTACACCTTCGGTTCG |
| 36 | NTGTCGGCTGCTCCCTCCCAGACATCTGTGTACTTCTGTGCCAGCAGTGAATTGGGACTCAAC |
| | ACAGATACGCAGTATTTTGGCCCA |
| 37 | NNNNNNNTGTCGGCTGCTCCCTCCCAGACATCTGTGTACTTCTGTGCCAGCAGTGCTATGGTC |
| | GAACAGCCCCAGCATTTTGGTGAT |
| 38 | NTGTCGGCTGCTCCCTCCCAGACATCTGTGTACTTCTGTGCCAGCAGTGAAATGGGACTCAAC |
| | ACAGATACGCAGTATTTTGGCCCA |
| 39 | CAGCGCACAGAGCAGGGGGACTCGGCCATGTATCTCTGTGCCAGCAGCCCTCGGGGGGCCGTG |
| | AACACTGAAGCTTTCTTTGGACAA |
| 40 | GAGTCTGCTGCCTCCTCCCAGACATCTGTATATTTCTGCGCCAGCAGTGAGTTAGGGACCAGA |
| | CCCAATGAGCAGTTCTTCGGGCCA |
| 41 | CGGTCCACAAAGCTGGAGGACTCAGCCATGTACTTCTGTGCCAGCAGGAAACAGGGTCTAGGA |
| | TACACTGAAGCTTTCTTTGGACAA |
| 42 | NNNNTGTCGGCTGCTCCCTCCCAAACATCTGTGTACTTCTGTGCCAGCAGTTACCGACAGACC |
| | AATCAGCCCCAGCATTTTGGTGAT |
| 43 | TCAACTCTGACTGTGAGCAACCTGAGCCCTGAAGACAGCAGCATATATCTCTGCAGCGTTCTA |
| | CAGCACGAGCAGTACTTCGGGCCG |
| 44 | ATCCAGCGCACACAGCAGGAGGACTCCGCCGTGTATCTCTGTGCCAGCAGTCCCCAGCCTAGC |
| | ACAGATACGCAGTATTTTGGCCCA |
| 45 | AAGATCCAGCGCACACAGCAGGAGGACTCCGCCGTGTATCTCTGTGCCAGCAGCGTCCGGGAT |
| | TATTCACCCCTCCACTTTGGGAAC |
| 46 | ATCCAGCGCACAGAGCAGGAGGACTCGGCCGTGTATCTCTGTGCCAGCAGCTTAGAGCGGGGG |
| | GCCTATGGCTACACCTTCGGTTCG |
| 47 | TCACCAGGCCTGGGGGACGCAGCCATGTACCTGTGTGCCACCAGCCAGGGGGTTGGCTCTGGG |
| | GCCAACGTCCTGACTTTCGGGGCC |
| 48 | ACAGTGACCAGTGCCCATCCTGAAGACAGCAGCTTCTACATCTGCAGTGCTACAGGGGGCACT |
| | AATGAAAAACTGTTTTTTGGCAGT |
| 49 | NNNNTGTCGGCTGCTCCCTCCCAGACATCTGTGTACTTCTGTGCCAGCAGGGAGACAGGGGGC |
| | GCCGGGGAGCTGTTTTTTGGAGAA |
| 50 | ACATCGGCCCAAAAGAACCCGACAGCTTTCTATCTCTGTGCCAGTAGTATAAGCCTAGCGCTA |
| | ATTTACGAGCAGTACTTCGGGCCG |
| 51 | CAGCGCACAGAGCAGGAGGACTCGGCCGTGTATCTCTGTGCCAGCAGCTTATATGCGGGCCAG |
| | AACACTGAAGCTTTCTTTGGACAA |
| 52 | AACGCCTTGTTGCTGGGGGACTCGGCCCTCTATCTCTGTGCCAGCAGCCCGGACTTCTCTGGG |
| | GCCAACGTCCTGACTTTCGGGGCC |
| 53 | CTGTCCTCAGAACCGGGAGACACGGCACTGTATCTCTGCGCCAGCAGTCAAACGGGACAGGGC |
| | TACAATGAGCAGTTCTTCGGGCCA |
| 54 | GCCATCCCCAACCAGACAGCTCTTTACTTCTGTGCCACCAGTGAAGGGGGCAGGGGGCGAGGA |
| | CCCGGGGAGCTGTTTTTTGGAGAA |
| 55 | AACGCCTTGGAGCTGGACGACTCGGCCCTGTATCTCTGTGCCAGCAGCTTGGACGGGAGCTCT |
| | GGAAACACCATATATTTTGGAGAG |
| 56 | CAGCCCTCAGAACCCAGGGACTCAGCTGTGTACTTCTGTGCCAGCAGTTTCCCCTCCCGGGGG |
| | AACACTGAAGCTTTCTTTGGACAA |
| 57 | CTGGAGTCAGCTACCCGCTCCCAGACATCTGTGTATTTCTGCGCCAGCACCTCAGGGGAAAGA |
| | GGCCCTGAAGCTTTCTTTGGACAA |
| 58 | AGCTCTCTGGAGCTGGGGGACTCAGCTTTGTATTTCTGTGCCAGCAGCCGCGGGACAGCGAAC |
| | ACCGGGGAGCTGTTTTTTGGAGAA |
| 59 | NNNNNNNTGTCGGCTGCTCCCTCCCAAACATCTGTGTACTTCTGTGCCTCTCTCTCAGGCCAC |
| | CAAGAGACCCAGTACTTCGGGCCA |
| 60 | CATCCTGAAGACAGCAGCTTCTACATCTGCAGTGCTAGAAACCGTATGGGCGGGACCTCCTCC |
| | TACAATGAGCAGTTCTTCGGGCCA |
| 61 | ACACAGCAGGAGGACTCCGCCGTGTATCTCTGTGCCAGCAGCTCCTCCCGACTAGCGGGGGTC |
| | ACAGATACGCAGTATTTTGGCCCA |
| 62 | AACGCCTTGTTGCTGGGGGACTCGGCCCTCTATCTCTGTGCCAGCAGCTTGAGCAGGGGGGTT |
| | TACTACGAGCAGTACTTCGGGCCG |
| 63 | TTGGAGCTGGACGACTCGGCCCTGTATCTCTGTGCCAGCAGCCAGACAGGCGGCAGGGGGCAG |
| | AATTCACCCCTCCACTTTGGGAAT |
| 64 | GTGAACGCCTTGGAGCTGGACGACTCGGCCCTGTATCTCTGTGCCAGCAGCGTGGGGGGTGGC |
| | CCCTACGAGCAGTACTTCGGGCCG |
| 65 | ATCCTGGAGTCGCCCAGCCCCAACCAGACCTCTCTGTACTTCTGTGCCAGCAGCAGACTAGCG |
| | GGCTACGAGCAGTACTTCGGGCCG |
| 66 | GTGACATCGGCCCAAAAGAACCCGACAGCTTTCTATCTCTGTGCCAGTCGATTAGCGGGAGCA |
| | GGAGATACGCAGTATTTTGGCCCA |
| 67 | ACAAAGCTGGAGGACTCAGCCATGTACTTCTGTGCCAGCAGTCCCCTTTCGGGACTAGCGTTA |
| | TACAATGAGCAGTTCTTCGGGCCA |
| 68 | ACCCTGCAGCCAGAAGACTCGGCCCTGTATCTCTGCGCCAGCAGGTTGTTAGGGGGCGAAGAT |
| | AGCACTGAAGCTTTCTTTGGACAA |
| 69 | GTGACCAGTGCCCATCCTGAAGACAGCAGCTTCTACATCTGCAGTGCCGTTTATCAGGGGGTG |
| | AACACTGAAGCTTTCTTTGGACAA |
| 70 | AGGATCCAGCAGGTAGTGCGAGGAGATTCGGCAGCTTATTTCTGTGCCAGCTCAGAGGGGGGG |
| | AATCAGCCCCAGCATTTTGGTGAT |
| 71 | ACTCTGGAGTCCGCTACCAGCTCCCAGACATCTGTGTACTTCTGTGCCATCAGTGAGGGGGCT |
| | GGGGAAAAACTGTTTTTTGGCAGT |
| 72 | CTGAAGATCCGGTCCACAAAGCTGGAGGACTCAGCCATGTACTTCTGTGCCTCGAGAGGGGGT |
| | GGCACTGAAGCTTTCTTTGGACAA |
| 73 | ACAGTGACCAGTGCCCATCCTGAAGACAGCAGCTTCTACATCTGCAGTGCTAGAGGGGATAGC |
| | TCCGGGGAGCTGTTTTTTGGAGAA |
| 74 | GCTACCAGCTCCCAGACATCTGTGTACTTCTGTGCCATCAGTGACCGAAGGACTAGCGAAAGC |
| | ACAGATACGCAGTATTTTGGCCCA |
| 75 | NNNNNNNTGTCGGCTGCTCCCTCCCAGACATCTGTGTACTTCTGTGCCAGCAGAGATCTAGGA |
| | GGGGTAGCCATATATTTTGGAGAG |
| 76 | ACTCTGACAGTGACCAGTGCCCATCCTGAAGACAGCAGCTTCTACATCTGCAGTGCTAAAAGA |
| | CACCACGAGCAGTACTTCGGGCCG |
| 77 | NNNNNNNNNNTGTCGGCTGCTCCCTCCCAGACATCTGTGTACTTCTGTGCCAGCAGCCGACCA |
| | GCAAGTGAGCAGTTCTTCGGGCCA |
| 78 | AACCTGAGCTCTCTGGAGCTGGGGGACTCAGCTTTGTATTTCTGTGCCAGCAGCGGTGTCAAC |
| | AACACTGAAGCTTTCTTTGGACAA |
| 79 | GTGAGCAACATGAGCCCTGAAGACAGCAGCATATATCTCTGCAGCGTTGAGTTTAGGGACAGG |
| | GACACTGAAGCTTTCTTTGGACAA |
| 80 | CTGACAGTGACCAGTGCCCATCCTGAAGACAGCAGCTTCTACATCTGCAGTGCCCGCAGCTCG |
| | GACACTGAAGCTTTCTTTGGACAA |
| 81 | ATTCTGGAGTCCGCCAGCACCAACCAGACATCTATGTACCTCTGTGCCAGCGTGACTAGCGGG |
| | ATCCATGAGCAGTTCTTCGGGCCA |
| 82 | ACAGTGACCAGTGCCCATCCTGAAGACAGCAGCTTCTACATCTGCAGTGCTAGCAAACAGAGT |
| | AACTATGGCTACACCTTCGGTTCG |
| 83 | AAGCTTGAGGACTCGGCCGTGTATCTCTGTGCCAGCAGCGACCAGCTACGACGGACTACAAGA |
| | AACTACGAGCAGTACTTCGGGCCG |
| 84 | CAGTCCACGGAGTCAGGGGACACAGCACTGTATTTCTGTGCCAGCAGCAAAGCGCTTTGGGTT |
| | ACGGGTGGCTACACCTTCGGTTCG |
| 85 | CACGCCCTGCAGCCAGAAGACTCAGCCCTGTATCTCTGCGCCAGCAGCCAAGACAGGGTGAAC |
| | ACCGGGGAGCTGTTTTTTGGAGAA |
| 86 | ACAGTGACCAGTGCCCATCCTGAAGACAGCAGCTTCTACATCTGCAGTGCTAGACGGGGCGAT |
| | AATTCACCCCTCCACTTTGGGAAT |
| 87 | NCTCTCACTGTGACATCGGCCCAAAAGAACCCGACAGCTTTCTATCTCTGTGCCAGTCAGACT |
| | CACAATGAGCAGTTCTTCGGGCCA |
| 88 | GAACTAAACCTGAGCTCTCTGGAGCTGGGGGACTCAGCTTTGTATTTCTGTGCCAGCAGCCCT |
| | CAAACTGAAGCTTTCTTTGGACAA |
| 89 | CAGCGCACACAGCAGGAGGACTCCGCCGTGTATCTCTGTGCCAGCAGCTCATTTCGGGGGGCC |
| | GGGGAGACCCAGTACTTCGGGCCA |
| 90 | GTGACCAGTGCCCATCCTGAAGACAGCAGCTTCTACATCTGCAGTGCTAGAAACCCGCTCGCT |
| | ACCTACGAGCAGTACTTCGGGCCG |
| 91 | GAGTCTGCCATCCCCAACCAGACAGCTCTTTACTTCTGTGCCACCAGTCAGGGGACACGACCT |
| | CTCTCAGCCCAGCATTTTGGTGAT |
| 92 | GCTGCTCCCTCCCAGACATCTGTGTACTTCTGTGCCAGCAGTCACAAAGGGACAGGGGTCCTT |
| | GATTCACCCCTCCACTTTGGGAAT |
| 93 | AATGTGAACGCCTTGTTGCTGGGGGACTCGGCCCTCTATCTCTGTGCCAGCAGCTTTACAGGT |
| | GGCGATGAGCAGTTCTTCGGGCCA |
| 94 | GCCTTGTTGCTGGGGGACTCGGCCCTCTATCTCTGTGCCAGCAGCTTAGGGCTAGCGGGAGGT |
| | CGAAACATTCAGTACTTCGGCGCC |
| 95 | AGTGCCCATCCTGAAGACAGCAGCTTCTACATCTGCAGTGCCCCAGCCAGAGGCGCCGAAACT |
| | TTCTACGAGCAGTACTTCGGGCCG |
| 96 | GTGAGCACCTTGGAGCTGGGGGACTCGGCCCTTTATCTTTGCGCCAGCATGGGGGATACTGGG |
| | GCCAACGTCCTGACTTTCGGGGCC |
| 97 | TTGGAGCTGGGGGACTCGGCCCTTTATCTTTGCGCCAGCAGCCCTCCCCGTGGGGGAGGGAAC |
| | ACCGGGGAGCTGTTTTTTGGAGAA |
| 98 | NNNNNNNTGTCGGCTGCTCCCTCCCAAACATCTGTGTACTTCTGTGCCAGCCGGAGGGACTTA |
| | AACTATGGCTACACCTTCGGTTCG |
| 99 | ATCAATTCCCTGGAGCTTGGTGACTCTGCTGTGTATTTCTGTGCCAGCAGCCAACGAGTACGG |
| | AATTCACCCCTCCACTTTGGGAAT |
| 100 | AGCTCTCTGGAGCTGGGGGACTCAGCTTTGTATTTCTGTGCCAGCACCGCCACAGGGCACTAT |
| | AATTCACCCCTCCACTTTGGGAAT |
| 101 | NNNNNNNTGTCGGCTGCTCCCTCCCAGACATCTGTGTACTTCTGTGCCAGCAGTGAGTTACGT |
| | AACAATGAGCAGTTCTTCGGGCCA |
| 102 | ACAGAGCAGGGGGACTCGGCCATGTATCTCTGTGCCAGCAGCGTCGTTAGGACGGACAGGGGG |
| | CGGGAGACCCAGTACTTCGGGCCA |
| 103 | ACCAGTGCCCATCCTGAAGACAGCAGCTTCTACATCTGCAGTGCCCGATTTGTACGGGGACGC |
| | AATCAGCCCCAGCATTTTGGTGAT |
| 104 | GTGAGCACCTTGGAGCTGGGGGACTCGGCCCTTTATCTTTGCGCCAGCGGGACAGCCCCGGAC |
| | AGAAATGAGCAGTTCTTCGGGCCA |
| 105 | ACTCTGGAGTCAGCTACCCGCTCCCAGACATCTGTGTATTTCTGCGCCAGCAGAGCAGGGGGA |
| | GATGAGACCCAGTACTTCGGGCCA |
| 106 | TTGTTGCTGGGGGACTCGGCCCTCTATCTCTGTGCCAGCAGCTTTGTAACCAGCGGGAGAGTA |
| | ACAGATACGCAGTATTTTGGCCCA |
| 107 | ATCCAGCCTGCAAAGCTTGAGGACTCGGCCGTGTATCTCTGTGCCAGCAGCTTAGAAAGGGGG |
| | TGGAATGAGCAGTTCTTCGGGCCA |
| 108 | CTTGAGGACTCGGCCGTGTATCTCTGTGCCAGCAGCTCCCCGAGACTAGCGGGAGGGCCTTCA |
| | GCCTACGAGCAGTACTTCGGGCCG |
| 109 | NNNNNNNTGTCGGCTGCTCCCTCCCAGACATCTGTGTACTTCTGTGCCAGCAGTGAAAGGGAG |
| | AATCAGCCCCAGCATTTTGGTGAT |
| 110 | AAGATCCGGTCCACAAAGCTGGAGGACTCAGCCATGTACTTCTGTGCCAGCAGTGTGAGACAG |
| | TCCTACGAGCAGTACTTCGGGCCG |
| 111 | GAGTCTGCCATCCCCAACCAGACAGCTCTTTACTTCTGTGCCACCAGGCCTGGACAGAGTTTG |
| | AACACTGAAGCTTTCTTTGGACAA |
| 112 | CTGGAGTCCGCCAGCACCAACCAGACATCTATGTACCTCTGTGCCAGCAGTTTATGGGGGGGC |
| | GATCAGCCCCAGCATTTTGGTGAT |
| 113 | CTGCAGCCAGAAGACTCGGCCCTGTATCTCTGCGCCAGCAGCCACAGAGGCAGCGGGAGAGCC |
| | CCAGATACGCAGTATTTTGGCCCA |
| 114 | NTGTCGGCTGCTCCCTCCCAGACATCTGTGTACTTCTGTGCCAGCAGGGGACTAGCGGGGAGC |
| | GGCGGGGAGCTGTTTTTTGGAGAA |
| 115 | GCCCAAAAGAACCCGACAGCTTTCTATCTCTGTGCCAGTAGTATAGATTCGACAGGGAAGGGG |
| | CCAGATACGCAGTATTTTGGCCCA |
| 116 | AGGATCCAGCAGGTAGTGCGAGGAGATTCGGCAGCTTATTTCTGTGCCAGCTCACCACCGAGT |
| | CGATATGGCTACACCTTCGGTTCG |
| 117 | TCACCAGGCCTGGGGGACGCAGCCATGTACCTGTGTGCCACCAGCAGCAGCTACAGGGGTAGG |
| | AGTCAGACCCAGTACTTCGGGCCA |
| 118 | CTGGAGTCGCCCAGCCCCAACCAGACCTCTCTGTACTTCTGTGCCAGCAGTCTAGCGGGGCTA |
| | GGCAATGAGCAGTTCTTCGGGCCA |
| 119 | TCCCTGGAGCTTGGTGACTCTGCTGTGTATTTCTGTGCCAGCAGCCAAGATGGGCTAGCGGGG |
| | TTTCTTGAGCAGTTCTTCGGGCCA |
| 120 | ACAGTGACCAGTGCCCATCCTGAAGACAGCAGCTTCTACATCTGCAGTGCGGGCGGGAGAGTT |
| | GCGGAGACCCAGTACTTCGGGCCA |
| 121 | TTGGCGTCTGCTGTACCCTCTCAGACATCTGTGTACTTCTGTGCCAGCAGTGAACAGGGAGTT |
| | TACCATGAGCAGTTCTTCGGGCCA |
| 122 | AAGCTCCTTCTCAGTGACTCTGGCTTCTATCTCTGTGCCTGGAGTACCCCGGGACTACGTAGC |
| | GTTGGTACGCAGTATTTTGGCCCA |
| 123 | GAGTCGCCCAGCCCCAACCAGACCTCTCTGTACTTCTGTGCCAGCAGTTCCCCGACAGGGAGG |
| | GACACTGAAGCTTTCTTTGGACAA |
| 124 | TCTAAGAAGCTCCTTCTCAGTGACTCTGGCTTCTATCTCTGTGCCTGGAGTGTACAGGGCAGG |
| | GGGAATGGCTACACCTTCGGTTCG |
| 125 | CTGACAGTGACCAGTGCCCATCCTGAAGACAGCAGCTTCTACATCTGCAGTCCGACTAGCGGG |
| | GAAGAGACCCAGTACTTCGGGCCA |
| 126 | ATCCAGCCCTCAGAACCCAGGGACTCAGCTGTGTACTTCTGTGCCAGCAGTTTGAGACAGGGA |
| | GCCCACGAGCAGTACTTCGGGCCG |
| 127 | TCGCCCAGCCCCAACCAGACCTCTCTGTACTTCTGTGCCAGCGGGGCCCGGACTAGCGGGAGA |
| | CGGGCGGTGCTGTTTTTTGGAGAA |
| 128 | ACCTTGGAGCTGGGGGACTCGGCCCTTTATCTTTGCGCCAGCAGCTTGGTACTAGCGGGCCGA |
| | AACAATGAGCAGTTCTTCGGGCCA |
| 129 | TCTAAGAAGCTCCTTCTCAGTGACTCTGGCTTCTATCTCTGTGCCTGGGAAGTGAAGGGACCC |
| | AATCAGCCCCAGCATTTTGGTGAT |
| 130 | TCCTCAGAACCGGGAGACACGGCACTGTATCTCTGCGCCAGCAGTCAATCCAGGCAGGAGGCT |
| | TCCTACGAGCAGTACTTCGGGCCG |
| 131 | CCAGAAGACTCGGCCCTGTATCTCTGCGCCAGCAGCCAAGGCGGGACTAGCGGGAGGGCCCTC |
| | CGAGATACGCAGTATTTTGGCCCA |
| 132 | CAGCGCACAGAGCAGGGGGACTCGGCCATGTATCTCTGTGCCAGCAGCCCCCAGAGGGGATTG |
| | AACACTGAAGCTTTCTTTGGACAA |
| 133 | GAGCAGGAGGACTCGGCCGTGTATCTCTGTGCCAGCAGCCCCGCCGGGGGAGTTGCGGCCAGC |
| | TCCTACGAGCAGTACTTCGGGCCG |
| 134 | ACAGAGCAGGGGGACTCGGCCATGTATCTCTGTGCCAGCAGCTTAGGTAATTGGCGGACCCCG |
| | AGCTATGGCTACACCTTCGGTTCG |
| 135 | CTGGCAATCCTGTCCTCAGAACCGGGAGACACGGCACTGTATCTCTGCGCCAGCAGTCACCCG |
| | AACTATGGCTACACCTTCGGTTCG |
| 136 | NNNNNNNTGTCGGCTGCTCCCTCCCAGACATCTGTGTACTTCTGTGCCAGCAGTCGGACAGGC |
| | AATCAGCCCCAGCATTTTGGTGAT |
| 137 | GTGACCAGTGCCCATCCTGAAGACAGCAGCTTCTACATCTGCAGTGCTAGAGAAGGGGGGTTC |
| | GGGAACGAGCAGTACTTCGGGCCG |
| 138 | AGGATCCAGCAGGTAGTGCGAGGAGATTCGGCAGCTTATTTCTGTGCCAGCGCCCAGGGGGGG |
| | GACACTGAAGCTTTCTTTGGACAA |
| 139 | ACTGTGACATCGGCCCAAAAGAACCCGACAGCTTTCTATCTCTGTGCCAGTAGTCCATTACAG |
| | ACCGGGGAGCTGTTTTTTGGAGAA |
| 140 | ACTCTGACAGTGACCAGTGCCCATCCTGAAGACAGCAGCTTCTACATCTGCAGTGCCCCAGGA |
| | GGGTATGGCTACACCTTCGGTTCG |
| 141 | ACAGTGACCAGTGCCCATCCTGAAGACAGCAGCTTCTACATCTGCAGTGCAAAAGACAGGGGG |
| | AAGAGGGGCTACACCTTCGGTTCG |
| 142 | TCTAAGAAGCTCCTTCTCAGTGACTCTGGCTTCTATCTCTGTGCCTGGACCTACCGGACGGGA |
| | GGAGTCGAGCAGTACTICGGGCCG |
| 143 | AGCACCTTGGAGCTGGGGGACTCGGCCCTTTATCTTTGCGCCAGCAGCTTGGACAGCAAAGCC |
| | AAAAACATTCAGTACTTCGGCGCC |
| 144 | TCAGAACCCAGGGACTCAGCTGTGTATTTTTGTGCTAGTGGTTTGAGGACTAGCGGGAAGTTG |
| | CGTAATGAGCAGTTCTTCGGGCCA |
| 145 | ATCCAGCGCACAGAGCAGGGGGACTCGGCCATGTATCTCTGTGCCAGCAGCTTAGGGGCTAGC |
| | GGGACTGAGCAGTTCTTCGGGCCA |
| 146 | AGCACCTTGGAGCTGGGGGACTCGGCCCTTTATCTTTGCGCCAGCAGCTCGGACAGGGGGCTA |
| | CACGAAAAACTGTTTTTTGGCAGT |
| 147 | GCTGCTCCCTCCCAGACATCTGTGTACTTCTGTGCCAGCAGCCTACGCGGCGGGGGGAATGGT |
| | TGGGATACGCAGTATTTTGGCCCA |
| 148 | CTAGAGTCTGCCATCCCCAACCAGACAGCTCTTTACTTCTGTGCCACCAGAGGGGGGCCGGAT |
| | GAAGAGACCCAGTACTTCGGGCCA |
| 149 | AACGCCTTGGAGCTGGACGACTCGGCCCTGTATCTCTGTGCCAGCAGCTCCCCGACAGGGGGT |
| | GACTACGAGCAGTACTTCGGGCCG |
| 150 | GTGAACGCCTTGTTGCTGGGGGACTCGGCCCTGTATCTCTGTGCCAGCAGCTTGTCCGGAAGT |
| | AATGAAAAACTGTTTTTTGGCAGT |
| 151 | CCTGCAAAGCTTGAGGACTCGGCCGTGTATCTCTGTGCCAGCAGCTTGAGGCGTCCGGGACAG |
| | GGGTGGGAGCTGTTTTTTGGAGAA |
| 152 | GAGTCCGCCAGCACCAACCAGACATCTATGTACCTCTGTGCCAGCAGTCTCGCGACAGGGGGC |
| | TTATCACCCCTCCACTTTGGGAAT |
| 153 | NNNNCTCTGAAGATCCGGTCCACAAAGCTGGAGGACTCAGCCATGTACTTCTGTGCCAGCAGG |
| | GATGATGGCTACACCTTCGGTTCG |
| 154 | AGCCCCAACCAGACCTCTCTGTACTTCTGTGCCAGCAGTTCCCCCGGACTAGCGGGAGGGAAC |
| | ACCGGGGAGCTGTTTTTTGGAGAA |
| 155 | GCTCCCTCCCAAACATCTGTGTACTTCTGTGCCAGCAGTTACTCCCCGGCGATCGGGGGGGTC |
| | TACAATGAGCAGTTCTTCGGGCCA |
| 156 | AGTGCCCATCCTGAAGACAGCAGCTTCTACATCTGCAGTGGAAAGGGACTAGCGGGAGGGGCT |
| | CAAGAGACCCAGTACTTCGGGCCA |
| 157 | GAGATCCAGCGCACAGAGCAGGGGGACTCGGCCATGTATCTCTGTGCCAGCACCCAGGGGGGG |
| | GTAGAGACCCAGTACTTCGGGCCA |
| 158 | CACACCCTGCAGCCAGAAGACTCGGCCCTGTATCTCTGCGCCAGCAGCCAAGAGCAGGGTCGC |
| | AATTCACCCCTCCACTTTGGGAAT |
| 159 | AATGTGAGCACCTTGGAGCTGGGGGACTCGGCCCTTTATCTTTGCGCCAGCAGCCAGTACAAT |
| | CACAATGAGCAGTTCTTCGGGCCA |
| 160 | ATCCAGCCCTCAGAACCCAGGGACTCAGCTGTGTATTTTTGTGCTAGTGGTTTGGCCCCCCTG |
| | AGCCGGGAGCAGTACTTCGGGCCG |
| 161 | CAGCCCTCAGAACCCAGGGACTCAGCTGTGTACTTCTGTGCCAGCAGTTTAGGGGGGTCGAGC |
| | AATCAGCCCCAGCATTTTGGTGAT |
| 162 | CAGCAGGTAGTGCGAGGAGATTCGGCAGCTTATTTCTGTGCCAGCTCACCCTGGCCCCAGGAT |
| | GGTGAAAAACTGTTTTTTGGCAGT |
| 163 | ACCAGTGCCCATCCTGAAGACAGCAGCTTCTACATCTGCAGTGCTAGAAAGGTGCAACGGGGC |
| | AAAAACATTCAGTACTTCGGCGCC |
| 164 | GTGACCAGTGCCCATCCTGAAGACAGCAGCTTCTACATCTGCAGTGCTAGAGCGGATGGACAG |
| | GGCTATGGCTACACCTTCGGTTCG |
| 165 | CACACCCTGCAGCCAGAAGACTCGGCCCTGTATCTCTGCGCCAGCAGCCAAGAGGTAGGGGGA |
| | GACCATGGCTACACCTTCGGTTCG |
| 166 | NNNNTGTCGGCTGCTCCCTCCCAGACATCTGTGTACTTCTGTGCCAGCATCCGCCCCGGTCTT |
| | AACTATGGCTACACCTTCGGTTCG |
| 167 | ATCCAGCAGGTAGTGCGAGGAGATTCGGCAGCTTATTTCTGTGCCAGCTCACCAGCTGACAGG |
| | GAAGAGACCCAGTACTTCGGGCCA |
| 168 | GAGTCTGCCATCCCCAACCAGACAGCTCTTTACTTCTGTGCCACCAGTGAACCGGGACCCTTA |
| | AATCAGCCCCAGCATTTTGGTGAT |
| 169 | GAGTCGCCCAGCCCCAACCAGACCTCTCTGTACTTCTGTGCCAGCAGAACCACGACAGGGTTC |
| | CTTTCACCCCTCCACTTTGGGAAC |
| 170 | ACAGTGACCAGTGCCCATCCTGAAGACAGCAGCTTCTACATCTGCAGTGGATTAGCGGGAGGC |
| | CAGGAGACCCAGTACTTCGGGCCA |
| 171 | NTGTCGGCTGCTCCCTCCCAGACATCTGTGTACTTCTGTGCCAGCAGCAAACCGGGACAGGGC |
| | TGGGAGACCCAGTACTTCGGGCCA |
| 172 | ATCCGCTCACCAGGCCTGGGGGACGCAGCCATGTACCTGTGTGCCACCAGCAGCAGCGGGTTC |
| | TCATACGAGCAGTACTTCGGGCCG |
| 173 | GCTGCTCCCTCCCAGACATCTGTGTACTTCTGTGCCAGCAGTTACATTGTGGGATTGACAACT |
| | AATGAAAAACTGTTTTTTGGCAGT |
| 174 | AGTTCTAAGAAGCTCCTTCTCAGTGACTCTGGCTTCTATCTCTGTGCCTGGGGGACTCTCTGG |
| | AGCGGGGAGCTGTTTTTTGGAGAA |
| 175 | TCCGCCAGCACCAACCAGACATCTATGTACCTCTGTGCCAGCCCCCCTGTTAGCGGGACAGAC |
| | TACAATGAGCAGTTCTTCGGGCCA |
| 176 | AACGCCTTGGAGCTGGACGACTCGGCCCTGTATCTCTGTGCCAGCAGCTTATACGACAGGCCC |
| | AGATATGGCTACACCTTCGGTTCG |
| 177 | ATTCTGGAGTCCGCCAGCACCAACCAGACATCTATGTACCTCTGTGCCAGCTACGGACAGGGG |
| | GTGCATGAGCAGTTCTTCGGGCCA |
| 178 | GTAGTGCGAGGAGATTCGGCAGCTTATTTCTGTGCCAGCTCACCACCGATCGGATTCACGACT |
| | AATGAAAAACTGTTTTTTGGCAGT |
| SEQ | |
| ID | |
| NO: | TCR Beta CDR3 |
| 179 | CASSIPQGSGSPLHF |
| 180 | CASSYGGGPPDTQYF |
| 181 | CASSQTALYSGNTIYF |
| 182 | CATALAGGHEQYF |
| 183 | CSAREGALDNSPLHF |
| 184 | CATSDFGKEGDEKLFF |
| 185 | CASSFTYDEQFF |
| 186 | CASSPGREPGNTIYF |
| 187 | CASTATESSPLHF |
| 188 | CASRKTGSLYGYTF |
| 189 | CASSLRTGIINEQFF |
| 190 | CASSTTGLAAYNEQFF |
| 191 | CASSFVDSPESYEQYF |
| 192 | CASSLTRIPEQYF |
| 193 | CSARVTGGGAHTGELFF |
| 194 | CASSLALGTGDTEAFF |
| 195 | CASSLAGTGTNEQFF |
| 196 | CASRGQRDTGELFF |
| 197 | CASSIRPNDEQFF |
| 198 | CASSYSSGGGNEQFF |
| 199 | CASRTIGQGSENYGYTE |
| 200 | CASSSLTPEAYGYTE |
| 201 | CSAMSPSGTGELFF |
| 202 | CASSDDLAANVLTF |
| 203 | CASSPRAGGAVREQYE |
| 204 | CAWSVGGPGANSPLHE |
| 205 | CASSKVNEKLFF |
| 206 | CASSLGGRSNQPQHE |
| 207 | CSAREPHDTSTDTQYF |
| 208 | CSASRTYDNQPQHF |
| 209 | CASSWDRTKEGNQPQHF |
| 210 | CASSSSGSNYNEQFF |
| 211 | CSARWYNEQFF |
| 212 | CASSPLLYSGNTIYF |
| 213 | CASSLPRDRDYGYTF |
| 214 | CASSELGLNTDTQYF |
| 215 | CASSAMVEQPQHE |
| 216 | CASSEMGLNTDTQYF |
| 217 | CASSPRGAVNTEAFF |
| 218 | CASSELGTRPNEQFF |
| 219 | CASRKQGLGYTEAFF |
| 220 | CASSYRQTNQPQHF |
| 221 | CSVLQHEQYF |
| 222 | CASSPQPSTDTQYF |
| 223 | CASSVRDYSPLHF |
| 224 | CASSLERGAYGYTF |
| 225 | CATSQGVGSGANVLTF |
| 226 | CSATGGTNEKLFF |
| 227 | CASRETGGAGELFF |
| 228 | CASSISLALIYEQYF |
| 229 | CASSLYAGQNTEAFF |
| 230 | CASSPDFSGANVLTE |
| 231 | CASSQTGQGYNEQFF |
| 232 | CATSEGGRGRGPGELFF |
| 233 | CASSLDGSSGNTIYF |
| 234 | CASSFPSRGNTEAFF |
| 235 | CASTSGERGPEAFF |
| 236 | CASSRGTANTGELFF |
| 237 | CASLSGHQETQYE |
| 238 | CSARNRMGGTSSYNEQFF |
| 239 | CASSSSRLAGVTDTQYF |
| 240 | CASSLSRGVYYEQYF |
| 241 | CASSQTGGRGQNSPLHF |
| 242 | CASSVGGGPYEQYF |
| 243 | CASSRLAGYEQYF |
| 244 | CASRLAGAGDTQYF |
| 245 | CASSPLSGLALYNEQFF |
| 246 | CASRLLGGEDSTEAFF |
| 247 | CSAVYQGVNTEAFF |
| 248 | CASSEGGNQPQHE |
| 249 | CAISEGAGEKLFF |
| 250 | CASRGGGTEAFF |
| 251 | CSARGDSSGELFF |
| 252 | CAISDRRTSESTDTQYE |
| 253 | CASRDLGGVAIYF |
| 254 | CSAKRHHEQYF |
| 255 | CASSRPASEQFF |
| 256 | CASSGVNNTEAFF |
| 257 | CSVEFRDRDTEAFF |
| 258 | CSARSSDTEAFF |
| 259 | CASVTSGIHEQFF |
| 260 | CSASKQSNYGYTE |
| 261 | CASSDQLRRTTRNYEQYF |
| 262 | CASSKALWVTGGYTE |
| 263 | CASSQDRVNTGELFF |
| 264 | CSARRGDNSPLHE |
| 265 | CASQTHNEQFF |
| 266 | CASSPQTEAFF |
| 267 | CASSSFRGAGETQYF |
| 268 | CSARNPLATYEQYF |
| 269 | CATSQGTRPLSAQHE |
| 270 | CASSHKGTGVLDSPLHF |
| 271 | CASSFTGGDEQFF |
| 272 | CASSLGLAGGRNIQYF |
| 273 | CSAPARGAETFYEQYF |
| 274 | CASMGDTGANVLTF |
| 275 | CASSPPRGGGNTGELFF |
| 276 | CASRRDLNYGYTE |
| 277 | CASSQRVRNSPLHE |
| 278 | CASTATGHYNSPLHF |
| 279 | CASSELRNNEQFF |
| 280 | CASSVVRTDRGRETQYF |
| 281 | CSARFVRGRNQPQHE |
| 282 | CASGTAPDRNEQFF |
| 283 | CASRAGGDETQYE |
| 284 | CASSFVTSGRVTDTQYF |
| 285 | CASSLERGWNEQFF |
| 286 | CASSSPRLAGGPSAYEQYF |
| 287 | CASSERENQPQHF |
| 288 | CASSVRQSYEQYF |
| 289 | CATRPGQSLNTEAFF |
| 290 | CASSLWGGDQPQHE |
| 291 | CASSHRGSGRAPDTQYE |
| 292 | CASRGLAGSGGELFF |
| 293 | CASSIDSTGKGPDTQYF |
| 294 | CASSPPSRYGYTF |
| 295 | CATSSSYRGRSQTQYF |
| 296 | CASSLAGLGNEQFF |
| 297 | CASSQDGLAGFLEQFF |
| 298 | CSAGGRVAETQYF |
| 299 | CASSEQGVYHEQFF |
| 300 | CAWSTPGLRSVGTQYF |
| 301 | CASSSPTGRDTEAFF |
| 302 | CAWSVQGRGNGYTE |
| 303 | CSPTSGEETQYF |
| 304 | CASSLRQGAHEQYF |
| 305 | CASGARTSGRRAVLFF |
| 306 | CASSLVLAGRNNEQFF |
| 307 | CAWEVKGPNQPQHE |
| 308 | CASSQSRQEASYEQYF |
| 309 | CASSQGGTSGRALRDTQYE |
| 310 | CASSPQRGLNTEAFF |
| 311 | CASSPAGGVAASSYEQYF |
| 312 | CASSLGNWRTPSYGYTF |
| 313 | CASSHPNYGYTE |
| 314 | CASSRTGNQPQHE |
| 315 | CSAREGGEGNEQYF |
| 316 | CASAQGGDTEAFF |
| 317 | CASSPLQTGELFF |
| 318 | CSAPGGYGYTE |
| 319 | CSAKDRGKRGYTF |
| 320 | CAWTYRTGGVEQYE |
| 321 | CASSLDSKAKNIQYF |
| 322 | CASGLRTSGKLRNEQFF |
| 323 | CASSLGASGTEQFF |
| 324 | CASSSDRGLHEKLFF |
| 325 | CASSLRGGGNGWDTQYF |
| 326 | CATRGGPDEETQYF |
| 327 | CASSSPTGGDYEQYF |
| 328 | CASSLSGSNEKLFF |
| 329 | CASSLRRPGQGWELFF |
| 330 | CASSLATGGLSPLHF |
| 331 | CASRDDGYTF |
| 332 | CASSSPGLAGGNTGELFF |
| 333 | CASSYSPAIGGVYNEQFF |
| 334 | CSGKGLAGGAQETQYF |
| 335 | CASTQGGVETQYF |
| 336 | CASSQEQGRNSPLHF |
| 337 | CASSQYNHNEQFF |
| 338 | CASGLAPLSREQYF |
| 339 | CASSLGGSSNQPQHF |
| 340 | CASSPWPQDGEKLFF |
| 341 | CSARKVQRGKNIQYF |
| 342 | CSARADGQGYGYTF |
| 343 | CASSQEVGGDHGYTF |
| 344 | CASIRPGLNYGYTF |
| 345 | CASSPADREETQYF |
| 346 | CATSEPGPLNQPQHF |
| 347 | CASRTTTGELSPLHF |
| 348 | CSGLAGGQETQYF |
| 349 | CASSKPGQGWETQYF |
| 350 | CATSSSGESYEQYF |
| 351 | CASSYIVGLTTNEKLFF |
| 352 | CAWGTLWSGELFF |
| 353 | CASPPVSGTDYNEQFF |
| 354 | CASSLYDRPRYGYTE |
| 355 | CASYGQGVHEQFF |
| 356 | CASSPPIGETTNEKLFF |
| SEQ | |
| ID | Immunodominant |
| NO: | peptides APOB |
| 358 | TLTAFGFASADLIEI |
| 360 | VEFVINMGIIIPDFA |
| 361 | VGSKLIVAMSSWLQK |
| 367 | LIINWLQEALSSASL |
| 368 | LEVLNEDFQANAQLS |
| 372 | ILFSYFQDLVITLPF |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown byway of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Kimura T, Kobiyama K, Winkels H, Tse K, Miller J, Vassallo M, et al. Regulatory cd4(+) t cells recognize mhc-ii-restricted peptide epitopes of apolipoprotein b. Circulation. 2018; 138:1130-1143.
2. Wolf D, Gerhardt T, Winkels H, Michel N A, Pramod A B, Ghosheh Y, et al. Pathogenic autoimmunity in atherosclerosis evolves from initially protective apolipoprotein b100-reactive cd4(+) t-regulatory cells. Circulation. 2020; 142:1279-1293.
3. Roy P, Orecchioni M, Ley K. How the immune system shapes atherosclerosis: Roles of innate and adaptive immunity. Nat Rev Immunol. 2021.
4. Sulzer D, Alcalay R N, Garretti F, Cote L, Kanter E, Agin-Liebes J, et al. T cells from patients with parkinson's disease recognize alpha-synuclein peptides. Nature. 2017; 546:656-661.
5. Gonzalez-Duque S, Azoury M E, Colli M L, Afonso G, Turatsinze J V, Nigi L, et al. Conventional and neo-antigenic peptides presented by beta cells are targeted by circulating naive cd8+t cells in type 1 diabetic and healthy donors. Cell Metab. 2018; 28:946-960 e946.
6. Zou J, Hannier S, Cairns L S, Barker R N, Rees A J, Turner A N, et al. Healthy individuals have goodpasture autoantigen-reactive t cells. J Am Soc Nephrol. 2008; 19:396-404
7. Legoux F P, Lim J B, Cauley A W, Dikiy S, Ertelt J, Mariani T J, et al. Cd4+t cell tolerance to tissue-restricted self antigens is mediated by antigen-specific regulatory t cells rather than deletion. Immunity. 2015; 43:896-908.
8. Malhotra D, Linehan J L, Dileepan T, Lee Y J, Purtha W E, Lu J V, et al. Tolerance is established in polyclonal cd4(+) t cells by distinct mechanisms, according to self-peptide expression patterns. Nat Immunol. 2016; 17:187-195.
9. Koltsova E K, Garcia Z, Chodaczek G, Landau M, McArdle S, Scott S R, et al. Dynamic t cell-apc interactions sustain chronic inflammation in atherosclerosis. J Clin Invest. 2012; 122:3114-3126.
10. Fernandez D M, Rahman A H, Fernandez N F, Chudnovskiy A, Amir E D, Amadori L, et al. Single-cell immune landscape of human atherosclerotic plaques. Nat Med. 2019; 25:1576-1588.
11. Depuydt M A C, Prange K H M, Slenders L, Ord T, Elbersen D, Boltjes A, et al. Microanatomy of the human atherosclerotic plaque by single-cell transcriptomics. Circ Res. 2020; 127:1437-1455.
12. Marchini T, Hansen S, Wolf D. Apob-specific cd4(+) t cells in mouse and human atherosclerosis. Cells. 2021; 10.
13. Nilsson J, Bjorkbacka H, Fredrikson G N. Apolipoprotein b100 autoimmunity and atherosclerosis—disease mechanisms and therapeutic potential. Curr Opin Lipidol. 2012; 23:422-428.
14. Fredrikson G N, Hedblad B, Berglund G, Alm R, Ares M, Cercek B, et al. Identification of immune responses against aldehyde-modified peptide sequences in apob associated with cardiovascular disease. Arterioscler Thromb Vase Biol. 2003; 23:872-878.
15. Frostegard J, Wu R, Giscombe R, Holm G, Lefvert A K, Nilsson J. Induction of t-cell activation by oxidized low density lipoprotein. Arterioscler Thromb. 1992; 12:461-467.
16. Gistera A, Klement M L, Polyzos K A, Mailer R K W, Duhlin A, Karlsson M C I, et al. Low-density lipoprotein-reactive t cells regulate plasma cholesterol levels and development of atherosclerosis in humanized hypercholesterolemic mice. Circulation. 2018; 138:2513-2526.
17. Prasad A, Clopton P, Ayers C, Khera A, de Lemos J A, Witztum J L, et al. Relationship of autoantibodies to mda-ldl and apob-immune complexes to sex, ethnicity, subclinical atherosclerosis, and cardiovascular events. Arterioscler Thromb Vase Biol. 2017; 37:1213-1221.
18. Tsiantoulas D, Diehl C J, Witztum J L, Binder C J. B cells and humoral immunity in atherosclerosis. Circ Res. 2014; 114:1743-1756.
19. Tsimikas S, Miyanohara A, Hartvigsen K, Merki E, Shaw P X, Chou M Y, et al. Human oxidation-specific antibodies reduce foam cell formation and atherosclerosis progression. J Am Coll Cardiol. 2011; 58:1715-1727.
20. Crotty S. A brief history of t cell help to b cells. Nat Rev Immunol. 2015; 15:185-189.
21. Robinson J, Barker D J, Georgiou X, Cooper M A, Flicek P, Marsh S G E. Ipd-imgt/hla database. Nucleic Acids Res. 2020; 48:D948-D955.
22. Attaf M, Huseby E, Sewell A K. Alphabeta t cell receptors as predictors of health and disease. Cell Mol Immunol. 2015; 12:391-399.
23. Turner S J, Doherty P C, McCluskey J, Rossjohn J. Structural determinants oft-cell receptor bias in immunity. Nat Rev Immunol. 2006; 6:883-894.
24. Ota K, Matsui M, Milford E L, Mackin G A, Weiner H L, Hafler D A. T-cell recognition of an immunodominant myelin basic protein epitope in multiple sclerosis. Nature. 1990; 346:183-187.
25. Purcell A W, Sechi S, DiLorenzo T P. The evolving landscape of autoantigen discovery and characterization in type 1 diabetes. Diabetes. 2019; 68:879-886.
26. Provenzano M, Panelli M C, Mocellin S, Bracci L, Sais G, Stroncek D F, et al. Mhc-peptide specificity and t-cell epitope mapping: Where immunotherapy starts. Trends Mol Med. 2006; 12:465-472.
27. Tian Y, da Silva Antunes R, Sidney J, Lindestam Arlehamn C S, Grifoni A, Dhanda S K, et al. A review on t cell epitopes identified using prediction and cell-mediated immune models for Mycobacterium tuberculosis and Bordetella pertussis. Front Immunol. 2018; 9:2778.
28. Hermansson A, Ketelhuth D F, Strodthoff D, Wurm M, Hansson E M, Nicoletti A, et al. Inhibition of t cell response to native low-density lipoprotein reduces atherosclerosis. J Exp Med. 2010; 207:1081-1093.
29. Gistera A, Hermansson A, Strodthoff D, Klement M L, Hedin U, Fredrikson G N, et al. Vaccination against t-cell epitopes of native apob100 reduces vascular inflammation and disease in a humanized mouse model of atherosclerosis. J Intern Med. 2017; 281:383-397.
30. Greenbaum J, Sidney J, Chung J, Brander C, Peters B, Sette A. Functional classification of class ii human leukocyte antigen (hla) molecules reveals seven different supertypes and a surprising degree of repertoire sharing across supertypes. Immunogenetics. 2011; 63:325-335.
31. Sidney J, Southwood S, Moore C, Oseroff C, Pinilla C, Grey H M, et al. Measurement of mhc/peptide interactions by gel filtration or monoclonal antibody capture. Curr Protoc Immunol. 2013; Chapter 18:Unit 18 13.
32. Paul S, Grifoni A, Peters B, Sette A. Major histocompatibility complex binding, eluted ligands, and immunogenicity: Benchmark testing and predictions. Front Immunol. 2019; 10:3151.
33. Vita R, Mahajan S, Overton J A, Dhanda S K, Martini S, Cantrell J R, et al. The immune epitope database (iedb): 2018 update. Nucleic Acids Res. 2019; 47:D339-D343.
34. da Silva Antunes R, Babor M, Carpenter C, Khalil N, Cortese M, Mentzer A J, et al. Th1/th17 polarization persists following whole-cell pertussis vaccination despite repeated acellular boosters. J Clin Invest. 2018; 128:3853-3865.
35. Dan J M, Havenar-Daughton C, Kendric K, Al-Kolla R, Kaushik K, Rosales S L, et al. Recurrent group a streptococcus tonsillitis is an immunosusceptibility disease involving antibody deficiency and aberrant tfh cells. Sci Transl Med. 2019; 11.
36. Grifoni A, Weiskopf D, Ramirez S I, Mateus J, Dan J M, Moderbacher C R, et al. Targets of t cell responses to sars-cov-2 coronavirus in humans with covid-19 disease and unexposed individuals. Cell. 2020; 181:1489-1501 e1415.
37. Mateus J, Grifoni A, Tarke A, Sidney J, Ramirez S I, Dan J M, et al. Selective and cross-reactive sars-cov-2 t cell epitopes in unexposed humans. Science. 2020; 370:89-94.
38. Frentsch M, Arbach O, Kirchhoff D, Moewes B, Worm M, Rothe M, et al. Direct access to cd4+t cells specific for defined antigens according to cd154 expression. Nat Med. 2005; 11:1118-1124.
39. Paul S, Arlehamn C S L, Schulten V, Westernberg L, Sidney J, Peters B, et al. Experimental validation of the rate tool for inferring hla restrictions of t cell epitopes. BMC Immunol. 2017; 18:20.
40. Paul S, Dillon M B C, Arlehamn C S L, Huang H, Davis M M, McKinney D M, et al. A population response analysis approach to assign class ii hla-epitope restrictions. J Immunol. 2015; 194:6164-6176.
41. McKinney D M, Southwood S, Hinz D, Oseroff C, Arlehamn C S, Schulten V, et al. A strategy to determine hla class ii restriction broadly covering the dr, dp, and dq allelic variants most commonly expressed in the general population. Immunogenetics. 2013; 65:357-370.
42. Robins H. Immunosequencing: Applications of immune repertoire deep sequencing. Curr Opin Immunol. 2013; 25:646-652.
43. Latorre D, Kallweit U, Armentani E, Foglierini M, Mele F, Cassotta A, et al. T cells in patients with narcolepsy target self-antigens of hypocretin neurons. Nature. 2018; 562:63-68.
44. Carlson C S, Emerson R O, Sherwood A M, Desmarais C, Chung M W, Parsons J M, et al. Using synthetic templates to design an unbiased multiplex per assay. Nat Commun. 2013; 4:2680.
45. Brady B L, Steinel N C, Bassing C H. Antigen receptor allelic exclusion: An update and reappraisal. J Immunol. 2010; 185:3801-3808.
46. Rosati E, Dowds C M, Liaskou E, Henriksen E K K, Karlsen T H, Franke A. Overview of methodologies for t-cell receptor repertoire analysis. BMC Biotechnol. 2017; 17:61.
47. Chiffelle J, Genolet R, Perez M A, Coukos G, Zoete V, Harari A. T-cell repertoire analysis and metrics of diversity and clonality. Curr Opin Biotechnol. 2020; 65:284-295.
48. Gensini G G. A more meaningful scoring system for determining the severity of coronary heart disease. Am J Cardiol. 1983; 51:606.
49. Lindestam Arlehamn C S, Pham J, Alcalay R N, Frazier A, Shorr E, Carpenter C, et al. Widespread tau-specific CD4 t cell reactivity in the general population. J Immunol. 2019; 203:84-92.
50. Goebels N, Hofstetter H, Schmidt S, Brunner C, Wekerle H, Hohlfeld R. Repertoire dynamics of autoreactive t cells in multiple sclerosis patients and healthy subjects: Epitope spreading versus clonal persistence. Brain. 2000; 123 Pt 3:508-518.
51. Musters A, Klarenbeek P L, Doorenspleet M E, Balzaretti G, Esveldt R E E, van Schaik B D C, et al. In rheumatoid arthritis, synovitis at different inflammatory sites is dominated by shared but patient-specific t cell clones. J Immunol. 2018; 201:417-422.
52. Risnes L F, Christophersen A, Dahal-Koirala S, Neumann R S, Sandve G K, Sarna V K, et al. Disease-driving cd4+t cell clonotypes persist for decades in celiac disease. J Clin Invest. 2018; 128:2642-2650.
53. Liu X, Zhang W, Zhao M, Fu L, Liu L, Wu J, et al. T cell receptor beta repertoires as novel diagnostic markers for systemic lupus erythematosus and rheumatoid arthritis. Ann Rheum Dis. 2019; 78:1070-1078.
54. Ogura H, Preston-Hurlburt P, Perdigoto A L, Amodio M, Krishnaswamy S, Clark P, et al. Identification and analysis of islet antigen-specific cd8(+) t cells with t cell libraries. J Immunol. 2018; 201:1662-1670.
55. Petersen J, Montserrat V, Mujico J R, Loh K L, Beringer D X, van Lummel M, et al. T-cell receptor recognition of hla-dq2-gliadin complexes associated with celiac disease. Nat Struct Mol Biol. 2014; 21:480-488.
56. Di Sante G, Tolusso B, Fedele A L, Gremese E, Alivernin S, Nicolo C, et al. Collagen specific t-cell repertoire and hla-dr alleles: Biomarkers of active refractory rheumatoid arthritis. EBioMedicine. 2015; 2:2037-2045.
57. Ostrov D A, Alkanani A, McDaniel K A, Case S, Baschal E E, Pyle L, et al. Methyldopa blocks mhc class ii binding to disease-specific antigens in autoimmune diabetes. J Clin Invest. 2018; 128:1888-1902.
58. Nikolic T, Zwaginga J J, Uitbeijerse B S, Woittiez N J, de Koning E J, Aanstoot H J, et al. Safety and feasibility of intradermal injection with tolerogenic dendritic cells pulsed with proinsulin peptide—for type 1 diabetes. Lancet Diabetes Endocrinol. 2020; 8:470-472
59. Hardy M Y, Goel G, Russell A K, Chen Yi Mei S L G, Brown G J E, Wang S, et al. A sensitive whole blood assay detects antigen-stimulated cytokine release from cd4+t cells and facilitates immunomonitoring in a phase 2 clinical trial of nexvax2 in coeliac disease. Front Immunol. 2021; 12:661622.

What is claimed is:

1. An engineered T cell receptor (TCR) comprising a human T cell beta chain with a CDR3 selected from the amino acid sequence of SEQ ID NOS: 179 to 356 and a human alpha chain, wherein the TCR is specific for a human apolipoprotein B (ApoB) epitope.

2. The TCR of claim 1, wherein the engineered TCR binds to a Class II HLA and a peptide selected from SEQ ID NOS: 358, 360, 361, 367, 368, or 372.

3. The TCR of claim 1, wherein the TCR comprises a beta chain CDR3 having at least 95, 96, 97, 98, or 99% identity to the amino acid sequence of SEQ ID NOS: 179 to 356.

4. The TCR of claim 1, wherein the TCR is humanized.

5. The TCR of claim 1, wherein the TCR comprises a beta chain having at least 90, 91, 92, 93, 94, 95, 96, 97, 98, 99, or 100% identity to the nucleotide sequence of SEQ ID NO: 1.

6. The TCR of claim 1, wherein the TCR is further defined as a soluble TCR, wherein the soluble TCR does not comprise a transmembrane domain, or comprises a transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.

7. The TCR of any one of claims 1-6, the TCR further comprising a detectable label.

8. The TCR of any one of claims 1-6, wherein the TCR is covalently bound to a therapeutic agent, an immunotoxin, or a chemotherapeutic agent.

9. The TCR of any one of claims 1-6, wherein the beta chain CDR3 is selected from SEQ ID NO: 179-232.

10. The TCR of any one of claims 1-6, wherein the TCR is part of a multivalent TCR complex comprising a plurality of TCRs according to claim 1.

11. The complex of claim 10, wherein the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule; or the human apolipoprotein B (ApoB) epitope is an immunodominant epitope.

12. A polypeptide encoding the TCR of claims 1-6.

13. A polynucleotide encoding the polypeptide of any one of claims 1-6.

14. An expression vector encoding the TCR of any one of claims 1-6.

15. The expression vector of claim 14, wherein the sequence encoding the TCR is under the control of a promoter.

16. The expression vector of claim 14, wherein the expression vector is a viral or a retroviral vector.

17. The expression vector of claim 14, wherein the vector further encodes a linker domain positioned between the alpha chain and beta chain.

18. The expression vector of claim 17, wherein the linker domain comprises one or more protease cleavage sites, or wherein the one or more cleavage sites are separated by a spacer.

19. A host cell engineered to express the TCR of any one of claims 1-6.

20. The host cell of claim 19, wherein the cell is a Treg cell, mesenchymal stem cell (MSC), or induced pluripotent stem (iPS) cell.

21. The host cell of claim 19, wherein the host cell is an immune cell.

22. The host cell of claim 19, wherein the host cell is a T cell that is a CD4⁺ T cell or γδ T cell.

23. The host cell of claim 19, wherein the host cell is a regulatory T cell (Treg) selected from a follicular regulatory T cell, a ST2 T reg, an activated T reg, or an effector Treg.

24. The host cell of claim 19, wherein the host cell is autologous or allogeneic.

25. A method for engineering a host cell comprising contacting an immune cell with the TCR of any one of claims 1-6 or the expression vector of any one of claims 14-18.

26. The method of claim 25, wherein contacting is further defined as transfecting or transducing, wherein transfecting comprises electroporating RNA encoding the TCR of any one of claims 1-8 into the immune cell.

27. A method for treating a subject with atherosclerosis with autoimmunity to human apolipoprotein B (APOB) peptides, the method comprising:
administering to the subject an effective amount of one or more immune cells modified by cloning genes of the alpha and beta chains of a T cell receptor (TCR) ex vivo to express a chimeric antigen receptor specific for the APOB peptide, wherein the chimeric antigen receptor comprises an alpha chain CDR3 and a beta chain CDR3 having the amino acid sequence of SEQ ID NO: 179 to 356.

28. The method of claim 27, wherein the immune cell is regulatory T cell (Treg) selected from a follicular regulatory T cell, a ST2 T reg, an activated T reg, or an effector Treg, mesenchymal stem cell (MSC), or induced pluripotent stem (iPS) cell, or a peripheral blood lymphocyte.

29. The method of claim 27, further comprising at least one of:
sorting the immune cells into T cells to isolate TCR engineered T cells;
performing a T cell cloning of the immune cells by serial dilution; or
expanding a T cell clone from the immune cells by a rapid expansion protocol.

30. The method of claim 27, wherein the subject is identified to have an HLA selected from:

| DPB1 | DQB1 | DRB1 | DRB3 | DRB4 | DRB5 |
|---|---|---|---|---|---|
| 04:01 + 11:01 | 03:03 + 05:01 | 09:01 + 10:01 | NP + NP | 01:01 + NP | NP + NP |
| 04:01 + 13:01 | 06:02 + 06:03 | 13:01 + 15:01 | 01:01 + NP | NP + NP | 01:01 + NP |
| 04:01 + 04:01 | 03:01 + 03:02 | 04:01 + 11:01 | 02:02 + NP | 01:01 + NP | NP + NP |
| 04:01 + 05:01 | 02:02 + 03:03 | 07:01 + 09:01 | NP + NP | 01:01 + 01:01 | NP + NP |
| 02:02 + 04:02 | 02:01 + 03:01 | 03:01 + 11:02 | 02:02 + 02:02 | NP + NP | NP + NP |
| 03:01 + 04:01 | 03:03 + 06:02 | 09:01 + 15:01 | NP + NP | 01:01 + NP | 01:01 + NP. |

31. The method of claim 27, wherein the immune cell is a T cell selected from a regulatory T cell (Treg) selected from a follicular regulatory T cell, a ST2 T reg, an activated T reg, or an effector Treg.

32. The method of claim 27, wherein the TCR engineered cells are autologous or allogeneic.

33. The method of claim 27, further comprising administering a second therapy selected from immunotherapy, surgery, or biotherapy.

34. The method of claim 27, wherein the one or more immune cells are administered intravenously, intraperitoneally, intratracheally, intramuscularly, endoscopically, intralesionally, percutaneously, subcutaneously, regionally, or by direct injection or perfusion.

35. A chimeric antigen receptor expressing T cell (CAR-T) comprising an antigen recognition moiety and a T-cell activation moiety, wherein the T-cell activation moiety comprises a transmembrane domain, and wherein the antigen recognition moiety comprises a T cell receptor beta chain CDR3 is selected from the amino acid sequence of SEQ ID NOS: 179 to 356 and an alpha chain, wherein the TCR is specific for a human apolipoprotein B (ApoB) epitope.

36. The CAR-T of claim 35, wherein the TCR binds to a Class II HLA and a peptide selected from SEQ ID NOS: 358, 360, 361, 367, 368, or 372.

37. The CAR-T of claim 35, wherein the transmembrane domain is a CD28 transmembrane domain or a CD8a transmembrane domain.

38. The CAR-T of claim 35, wherein the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.

39. The CAR-T of claim 35, wherein the TCR comprises a beta chain having at least 90, 91, 92, 93, 94, 95, 96, 97, 98, 99, or 100% identity to the nucleotide sequence of SEQ ID NO: 1.

40. The CAR-T of claim 35, wherein the antigen recognition moiety comprises a beta chain CDR3 having an amino acid sequence selected from one of SEQ ID NO: 179-232.

41. The CAR-T of claim 35, wherein the immune cell is a T cell selected from a regulatory T cell (Treg) selected from a follicular regulatory T cell, a ST2 T reg, an activated T reg, or an effector Treg.


SEQUENCE LISTING (376 sequences)

SEQ ID NO: 1 - DNA, 87 bp, synthetic construct
  misc_feature 1..87: note = Synthetic
  source 1..87: mol_type = other DNA
aacgccttgttgctgggggactcggccctctatctctgtgccagcagcatcccacagggttctggttcacccctccactttgggaat

SEQ ID NO: 2 - DNA, 87 bp, synthetic construct
  misc_feature 1..87: note = Synthetic
  source 1..87: mol_type = other DNA
gagttggctgctccctcccagacatctgtgtacttctgtgccagcagttacgggggagggccgccagatacgcagtattttggccca

SEQ ID NO: 3 - DNA, 87 bp, synthetic construct
  misc_feature 1..87: note = Synthetic
  source 1..87: mol_type = other DNA
tcggcccaaaagaacccgacagctttctatctctgtgccagtagtcaaaccgctctttactctggaaacaccatatattttggagag

SEQ ID NO: 4 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccaccgccttagcgggaggacacgagcagtacttcgggccg

SEQ ID NO: 5 - DNA, 87 bp, synthetic construct
  misc_feature 1..87: note = Synthetic
  source 1..87: mol_type = other DNA
accagtgcccatcctgaagacagcagcttctacatctgcagtgctagagaaggggcacttgataattcacccctccactttgggaat

SEQ ID NO: 6 - DNA, 87 bp, synthetic construct
  misc_feature 1..87: note = Synthetic
  source 1..87: mol_type = other DNA
tctgccatccccaaccagacagctctttacttctgtgccaccagtgatttcgggaaagaaggggatgaaaaactgttttttggcagt

SEQ ID NO: 7 - DNA, 87 bp, synthetic construct
  misc_feature 1..87: note = Synthetic
  source 1..87: mol_type = other DNA
ttggagatccagcgcacagagcagggggactcggccatgtatctctgtgccagcagctttacttatgatgagcagttcttcgggcca

SEQ ID NO: 8 - DNA, 87 bp, synthetic construct
  misc_feature 1..87: note = Synthetic
  source 1..87: mol_type = other DNA
cagcgcacagagcagggggactcggccatgtatctctgtgccagcagccccggaagggaacctggaaacaccatatattttggagag

SEQ ID NO: 9 - DNA, 87 bp, synthetic construct
  misc_feature 1..87: note = Synthetic
  source 1..87: mol_type = other DNA
aagatccagccctcagaacccagggactcagctgtgtacttctgtgccagcacggccacggagagctcacccctccactttgggaat

SEQ ID NO: 10 - DNA, 87 bp, synthetic construct
  misc_feature 1..87: note = Synthetic
  source 1..87: mol_type = other DNA
ctggagtcgcccagccccaaccagacctctctgtacttctgtgccagccgaaaaacgggaagtttatatggctacaccttcggttcg

SEQ ID NO: 11 - DNA, 87 bp, synthetic construct
  misc_feature 1..87: note = Synthetic
  source 1..87: mol_type = other DNA
agcaccttggagctgggggactcggccctttatctttgcgccagcagcttaaggacagggataatcaatgagcagttcttcgggcca

SEQ ID NO: 12 - DNA, 87 bp, synthetic construct
  misc_feature 1..87: note = Synthetic
  source 1..87: mol_type = other DNA
tcggcccaaaagaacccgacagctttctatctctgtgccagtagtaccacgggactagcggcatacaatgagcagttcttcgggcca

SEQ ID NO: 13 - DNA, 87 bp, synthetic construct
  misc_feature 1..87: note = Synthetic
  source 1..87: mol_type = other DNA
ccctcagaacccagggactcagctgtgtacttctgtgccagcagttttgtggacagccccgaatcctacgagcagtacttcgggccg

SEQ ID NO: 14 - DNA, 87 bp, synthetic construct
  misc_feature 1..87: note = Synthetic
  source 1..87: mol_type = other DNA
attctggagtccgccagcaccaaccagacatctatgtacctctgtgccagcagtttaacccggattcccgagcagtacttcgggccg

SEQ ID NO: 15 - DNA, 87 bp, synthetic construct
  misc_feature 1..87: note = Synthetic
  source 1..87: mol_type = other DNA
gcccatcctgaagacagcagcttctacatctgcagtgctagagtaacaggcgggggggcccacaccggggagctgttttttggagaa

SEQ ID NO: 16 - DNA, 87 bp, synthetic construct
  misc_feature 1..87: note = Synthetic
  source 1..87: mol_type = other DNA
accttggagctgggggactcggccctttatctttgcgccagcagcttggcgcttgggacaggggacactgaagctttctttggacaa

SEQ ID NO: 17 - DNA, 87 bp, synthetic construct
  misc_feature 1..87: note = Synthetic
  source 1..87: mol_type = other DNA
agcaccttggagctgggggactcggccctttatctttgcgccagcagcttggcggggacagggacaaatgagcagttcttcgggcca

SEQ ID NO: 18 - DNA, 87 bp, synthetic construct
  misc_feature 1..87: note = Synthetic
  source 1..87: mol_type = other DNA
gtgacatcggcccaaaagaacccgacagctttctatctctgtgccagtagagggcagagggacaccggggagctgttttttggagaa

SEQ ID NO: 19 - DNA, 87 bp, synthetic construct
  misc_feature 1..87: note = Synthetic
  source 1..87: mol_type = other DNA
aacctgagctctctggagctgggggactcagctttgtatttctgtgccagcagcatccgacccaacgatgagcagttcttcgggcca

SEQ ID NO: 20 - DNA, 87 bp, synthetic construct
  misc_feature 1..87: note = Synthetic
  variation 1: note = n is a, c, g, or t
  source 1..87: mol_type = other DNA
ntgtcggctgctccctcccagacatctgtgtacttctgtgccagcagttactcgagcgggggaggcaatgagcagttcttcgggcca

SEQ ID NO: 21 - DNA, 87 bp, synthetic construct
  misc_feature 1..87: note = Synthetic
  source 1..87: mol_type = other DNA
cccagccccaaccagacctctctgtacttctgtgccagcagaaccataggacaggggtcctttaactatggctacaccttcggttcg

SEQ ID NO: 22 - DNA, 87 bp, synthetic construct
  misc_feature 1..87: note = Synthetic
  source 1..87: mol_type = other DNA
gagtccgccagcaccaaccagacatctatgtacctctgtgccagcagttccttaaccccggaggcctatggctacaccttcggttcg

SEQ ID NO: 23 - DNA, 87 bp, synthetic construct
  misc_feature 1..87: note = Synthetic
  source 1..87: mol_type = other DNA
gtgaccagtgcccatcctgaagacagcagcttctacatctgcagtgctatgtcccctagcgggaccggggagctgttttttggagaa

SEQ ID NO: 24 - DNA, 87 bp, synthetic construct
  misc_feature 1..87: note = Synthetic
  source 1..87: mol_type = other DNA
atgagctccttggagctgggggactcagccctgtacttctgtgccagcagcgacgatctggcggccaacgtcctgactttcggggcc

SEQ ID NO: 25 - DNA, 87 bp, synthetic construct
  misc_feature 1..87: note = Synthetic
  source 1..87: mol_type = other DNA
gccttggagctggaggactcggccctgtatctctgtgccagcagccctagagcagggggcgccgtgagagagcagtacttcgggccg

SEQ ID NO: 26 - DNA, 87 bp, synthetic construct
  misc_feature 1..87: note = Synthetic
  source 1..87: mol_type = other DNA
aagctccttctcagtgactctggcttctatctctgtgcctggagtgtgggaggtcctggagccaattcacccctccactttgggaac

SEQ ID NO: 27 - DNA, 87 bp, synthetic construct
  misc_feature 1..87: note = Synthetic
  source 1..87: mol_type = other DNA
cttcacatcaattccctggagcttggtgactctgctgtgtatttctgtgccagcagcaaagttaatgaaaaactgttttttggcagt

SEQ ID NO: 28 - DNA, 87 bp, synthetic construct
  misc_feature 1..87: note = Synthetic
  source 1..87: mol_type = other DNA
agcaccttggagctgggggactcggccctttatctttgcgccagcagcttgggaggtcggagcaatcagccccagcattttggtgat

SEQ ID NO: 29 - DNA, 87 bp, synthetic construct
  misc_feature 1..87: note = Synthetic
  source 1..87: mol_type = other DNA
agtgcccatcctgaagacagcagcttctacatctgcagtgctagagaaccccatgacacgagcacagatacgcagtattttggccca

SEQ ID NO: 30 - DNA, 87 bp, synthetic construct
  misc_feature 1..87: note = Synthetic
  source 1..87: mol_type = other DNA
gtgaccagtgcccatcctgaagacagcagcttctacatctgcagtgctagtcgtacgtatgacaatcagccccagcattttggtgat

SEQ ID NO: 31 - DNA, 87 bp, synthetic construct
  misc_feature 1..87: note = Synthetic
  source 1..87: mol_type = other DNA
gcccaaaagaacccgacagctttctatctctgtgccagtagttgggacaggaccaagtttggcaatcagccccagcattttggtgat

SEQ ID NO: 32 - DNA, 87 bp, synthetic construct
  misc_feature 1..87: note = Synthetic
  source 1..87: mol_type = other DNA
aattccctggagcttggtgactctgctgtgtatttctgtgccagcagctctagcgggagtaactacaatgagcagttcttcgggcca

SEQ ID NO: 33 - DNA, 87 bp, synthetic construct
  misc_feature 1..87: note = Synthetic
  source 1..87: mol_type = other DNA
actctgacagtgaccagtgcccatcctgaagacagcagcttctacatctgcagtgctagatggtacaatgagcagttcttcgggcca

SEQ ID NO: 34 - DNA, 87 bp, synthetic construct
  misc_feature 1..87: note = Synthetic
  source 1..87: mol_type = other DNA
cagcgcacagagcaggaggactcggccgtgtatctctgtgccagcagccccctcctgtactctggaaacaccatatattttggagag

SEQ ID NO: 35 - DNA, 87 bp, synthetic construct
  misc_feature 1..87: note = Synthetic
  variation 1: note = n is a, c, g, or t
  source 1..87: mol_type = other DNA
ntgtcggctgctccctcccaaacatctgtgtacttctgtgccagcagcctaccccgggacagggactatggctacaccttcggttcg

SEQ ID NO: 36 - DNA, 87 bp, synthetic construct
  misc_feature 1..87: note = Synthetic
  variation 1: note = n is a, c, g, or t
  source 1..87: mol_type = other DNA
ntgtcggctgctccctcccagacatctgtgtacttctgtgccagcagtgaattgggactcaacacagatacgcagtattttggccca

SEQ ID NO: 37 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccagcagtgctatggtcgaacagccccagcattttggtgat

SEQ ID NO: 38 - DNA, 87 bp, synthetic construct
  misc_feature 1..87: note = Synthetic
  variation 1: note = n is a, c, g, or t
  source 1..87: mol_type = other DNA
ntgtcggctgctccctcccagacatctgtgtacttctgtgccagcagtgaaatgggactcaacacagatacgcagtattttggccca

SEQ ID NO: 39 - DNA, 87 bp, synthetic construct
  misc_feature 1..87: note = Synthetic
  source 1..87: mol_type = other DNA
cagcgcacagagcagggggactcggccatgtatctctgtgccagcagccctcggggggccgtgaacactgaagctttctttggacaa

SEQ ID NO: 40 - DNA, 87 bp, synthetic construct
  misc_feature 1..87: note = Synthetic
  source 1..87: mol_type = other DNA
gagtctgctgcctcctcccagacatctgtatatttctgcgccagcagtgagttagggaccagacccaatgagcagttcttcgggcca

SEQ ID NO: 41 - DNA, 87 bp, synthetic construct
  misc_feature 1..87: note = Synthetic
  source 1..87: mol_type = other DNA
cggtccacaaagctggaggactcagccatgtacttctgtgccagcaggaaacagggtctaggatacactgaagctttctttggacaa

SEQ ID NO: 42 - DNA, 87 bp, synthetic construct
  misc_feature 1..87: note = Synthetic
  variation 1..4: note = n is a, c, g, or t
  source 1..87: mol_type = other DNA
nnnntgtcggctgctccctcccaaacatctgtgtacttctgtgccagcagttaccgacagaccaatcagccccagcattttggtgat

SEQ ID NO: 43 - DNA, 87 bp, synthetic construct
  misc_feature 1..87: note = Synthetic
  source 1..87: mol_type = other DNA
tcaactctgactgtgagcaacctgagccctgaagacagcagcatatatctctgcagcgttctacagcacgagcagtacttcgggccg

SEQ ID NO: 44 - DNA, 87 bp, synthetic construct
  misc_feature 1..87: note = Synthetic
  source 1..87: mol_type = other DNA
atccagcgcacacagcaggaggactccgccgtgtatctctgtgccagcagtccccagcctagcacagatacgcagtattttggccca

SEQ ID NO: 45 - DNA, 87 bp, synthetic construct
  misc_feature 1..87: note = Synthetic
  source 1..87: mol_type = other DNA
aagatccagcgcacacagcaggaggactccgccgtgtatctctgtgccagcagcgtccgggattattcacccctccactttgggaac

SEQ ID NO: 46 - DNA, 87 bp, synthetic construct
  misc_feature 1..87: note = Synthetic
  source 1..87: mol_type = other DNA
atccagcgcacagagcaggaggactcggccgtgtatctctgtgccagcagcttagagcggggggcctatggctacaccttcggttcg

SEQ ID NO: 47 - DNA, 87 bp, synthetic construct
  misc_feature 1..87: note = Synthetic
  source 1..87: mol_type = other DNA
tcaccaggcctgggggacgcagccatgtacctgtgtgccaccagccagggggttggctctggggccaacgtcctgactttcggggcc

SEQ ID NO: 48 - DNA, 87 bp, synthetic construct
  misc_feature 1..87: note = Synthetic
  source 1..87: mol_type = other DNA
acagtgaccagtgcccatcctgaagacagcagcttctacatctgcagtgctacagggggcactaatgaaaaactgttttttggcagt

SEQ ID NO: 49 - DNA, 87 bp, synthetic construct
  misc_feature 1..87: note = Synthetic
  variation 1..4: note = n is a, c, g, or t
  source 1..87: mol_type = other DNA
nnnntgtcggctgctccctcccagacatctgtgtacttctgtgccagcagggagacagggggcgccggggagctgttttttggagaa

SEQ ID NO: 50 - DNA, 87 bp, synthetic construct
  misc_feature 1..87: note = Synthetic
  source 1..87: mol_type = other DNA
acatcggcccaaaagaacccgacagctttctatctctgtgccagtagtataagcctagcgctaatttacgagcagtacttcgggccg

SEQ ID NO: 51 - DNA, 87 bp, synthetic construct
  misc_feature 1..87: note = Synthetic
  source 1..87: mol_type = other DNA
cagcgcacagagcaggaggactcggccgtgtatctctgtgccagcagcttatatgcgggccagaacactgaagctttctttggacaa

SEQ ID NO: 52 - DNA, 87 bp, synthetic construct
  misc_feature 1..87: note = Synthetic
  source 1..87: mol_type = other DNA
aacgccttgttgctgggggactcggccctctatctctgtgccagcagcccggacttctctggggccaacgtcctgactttcggggcc

SEQ ID NO: 53 - DNA, 87 bp, synthetic construct
  misc_feature 1..87: note = Synthetic
  source 1..87: mol_type = other DNA
ctgtcctcagaaccgggagacacggcactgtatctctgcgccagcagtcaaacgggacagggctacaatgagcagttcttcgggcca

SEQ ID NO: 54 - DNA, 87 bp, synthetic construct
  misc_feature 1..87: note = Synthetic
  source 1..87: mol_type = other DNA
gccatccccaaccagacagctctttacttctgtgccaccagtgaagggggcagggggcgaggacccggggagctgttttttggagaa

SEQ ID NO: 55 - DNA, 87 bp, synthetic construct
  misc_feature 1..87: note = Synthetic
  source 1..87: mol_type = other DNA
aacgccttggagctggacgactcggccctgtatctctgtgccagcagcttggacgggagctctggaaacaccatatattttggagag

SEQ ID NO: 56 - DNA, 87 bp, synthetic construct
  misc_feature 1..87: note = Synthetic
  source 1..87: mol_type = other DNA
cagccctcagaacccagggactcagctgtgtacttctgtgccagcagtttcccctcccgggggaacactgaagctttctttggacaa

SEQ ID NO: 57 - DNA, 87 bp, synthetic construct
  misc_feature 1..87: note = Synthetic
  source 1..87: mol_type = other DNA
ctggagtcagctacccgctcccagacatctgtgtatttctgcgccagcacctcaggggaaagaggccctgaagctttctttggacaa

SEQ ID NO: 58 - DNA, 87 bp, synthetic construct
  misc_feature 1..87: note = Synthetic
  source 1..87: mol_type = other DNA
agctctctggagctgggggactcagctttgtatttctgtgccagcagccgcgggacagcgaacaccggggagctgttttttggagaa

SEQ ID NO: 59 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccaaacatctgtgtacttctgtgcctctctctcaggccaccaagagacccagtacttcgggcca

SEQ ID NO: 60 - DNA, 87 bp, synthetic construct
  misc_feature 1..87: note = Synthetic
  source 1..87: mol_type = other DNA
catcctgaagacagcagcttctacatctgcagtgctagaaaccgtatgggcgggacctcctcctacaatgagcagttcttcgggcca

SEQ ID NO: 61 - DNA, 87 bp, synthetic construct
  misc_feature 1..87: note = Synthetic
  source 1..87: mol_type = other DNA
acacagcaggaggactccgccgtgtatctctgtgccagcagctcctcccgactagcgggggtcacagatacgcagtattttggccca

SEQ ID NO: 62 - DNA, 87 bp, synthetic construct
  misc_feature 1..87: note = Synthetic
  source 1..87: mol_type = other DNA
aacgccttgttgctgggggactcggccctctatctctgtgccagcagcttgagcaggggggtttactacgagcagtacttcgggccg

SEQ ID NO: 63 - DNA, 87 bp, synthetic construct
  misc_feature 1..87: note = Synthetic
  source 1..87: mol_type = other DNA
ttggagctggacgactcggccctgtatctctgtgccagcagccagacaggcggcagggggcagaattcacccctccactttgggaat

SEQ ID NO: 64 - DNA, 87 bp, synthetic construct
  misc_feature 1..87: note = Synthetic
  source 1..87: mol_type = other DNA
gtgaacgccttggagctggacgactcggccctgtatctctgtgccagcagcgtggggggtggcccctacgagcagtacttcgggccg

SEQ ID NO: 65 - DNA, 87 bp, synthetic construct
  misc_feature 1..87: note = Synthetic
  source 1..87: mol_type = other DNA
atcctggagtcgcccagccccaaccagacctctctgtacttctgtgccagcagcagactagcgggctacgagcagtacttcgggccg

SEQ ID NO: 66 - DNA, 87 bp, synthetic construct
  misc_feature 1..87: note = Synthetic
  source 1..87: mol_type = other DNA
gtgacatcggcccaaaagaacccgacagctttctatctctgtgccagtcgattagcgggagcaggagatacgcagtattttggccca

SEQ ID NO: 67 - DNA, 87 bp, synthetic construct
  misc_feature 1..87: note = Synthetic
  source 1..87: mol_type = other DNA
acaaagctggaggactcagccatgtacttctgtgccagcagtcccctttcgggactagcgttatacaatgagcagttcttcgggcca

SEQ ID NO: 68 - DNA, 87 bp, synthetic construct
  misc_feature 1..87: note = Synthetic
  source 1..87: mol_type = other DNA
accctgcagccagaagactcggccctgtatctctgcgccagcaggttgttagggggcgaagatagcactgaagctttctttggacaa

SEQ ID NO: 69 - DNA, 87 bp, synthetic construct
  misc_feature 1..87: note = Synthetic
  source 1..87: mol_type = other DNA
gtgaccagtgcccatcctgaagacagcagcttctacatctgcagtgccgtttatcagggggtgaacactgaagctttctttggacaa

SEQ ID NO: 70 - DNA, 87 bp, synthetic construct
  misc_feature 1..87: note = Synthetic
  source 1..87: mol_type = other DNA
aggatccagcaggtagtgcgaggagattcggcagcttatttctgtgccagctcagagggggggaatcagccccagcattttggtgat

SEQ ID NO: 71 - DNA, 87 bp, synthetic construct
  misc_feature 1..87: note = Synthetic
  source 1..87: mol_type = other DNA
actctggagtccgctaccagctcccagacatctgtgtacttctgtgccatcagtgagggggctggggaaaaactgttttttggcagt

SEQ ID NO: 72 - DNA, 87 bp, synthetic construct
  misc_feature 1..87: note = Synthetic
  source 1..87: mol_type = other DNA
ctgaagatccggtccacaaagctggaggactcagccatgtacttctgtgcctcgagagggggtggcactgaagctttctttggacaa

SEQ ID NO: 73 - DNA, 87 bp, synthetic construct
  misc_feature 1..87: note = Synthetic
  source 1..87: mol_type = other DNA
acagtgaccagtgcccatcctgaagacagcagcttctacatctgcagtgctagaggggatagctccggggagctgttttttggagaa

SEQ ID NO: 74 - DNA, 87 bp, synthetic construct
  misc_feature 1..87: note = Synthetic
  source 1..87: mol_type = other DNA
gctaccagctcccagacatctgtgtacttctgtgccatcagtgaccgaaggactagcgaaagcacagatacgcagtattttggccca

SEQ ID NO: 75 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccagcagagatctaggaggggtagccatatattttggagag

SEQ ID NO: 76 - DNA, 87 bp, synthetic construct
  misc_feature 1..87: note = Synthetic
  source 1..87: mol_type = other DNA
actctgacagtgaccagtgcccatcctgaagacagcagcttctacatctgcagtgctaaaagacaccacgagcagtacttcgggccg

SEQ ID NO: 77 - DNA, 87 bp, synthetic construct
  misc_feature 1..87: note = Synthetic
  variation 1..10: note = n is a, c, g, or t
  source 1..87: mol_type = other DNA
nnnnnnnnnntgtcggctgctccctcccagacatctgtgtacttctgtgccagcagccgaccagcaagtgagcagttcttcgggcca

SEQ ID NO: 78 - DNA, 87 bp, synthetic construct
  misc_feature 1..87: note = Synthetic
  source 1..87: mol_type = other DNA
aacctgagctctctggagctgggggactcagctttgtatttctgtgccagcagcggtgtcaacaacactgaagctttctttggacaa

SEQ ID NO: 79 - DNA, 87 bp, synthetic construct
  misc_feature 1..87: note = Synthetic
  source 1..87: mol_type = other DNA
gtgagcaacatgagccctgaagacagcagcatatatctctgcagcgttgagtttagggacagggacactgaagctttctttggacaa

SEQ ID NO: 80 - DNA, 87 bp, synthetic construct
  misc_feature 1..87: note = Synthetic
  source 1..87: mol_type = other DNA
ctgacagtgaccagtgcccatcctgaagacagcagcttctacatctgcagtgcccgcagctcggacactgaagctttctttggacaa

SEQ ID NO: 81 - DNA, 87 bp, synthetic construct
  misc_feature 1..87: note = Synthetic
  source 1..87: mol_type = other DNA
attctggagtccgccagcaccaaccagacatctatgtacctctgtgccagcgtgactagcgggatccatgagcagttcttcgggcca

SEQ ID NO: 82 - DNA, 87 bp, synthetic construct
  misc_feature 1..87: note = Synthetic
  source 1..87: mol_type = other DNA
acagtgaccagtgcccatcctgaagacagcagcttctacatctgcagtgctagcaaacagagtaactatggctacaccttcggttcg

SEQ ID NO: 83 - DNA, 87 bp, synthetic construct
  misc_feature 1..87: note = Synthetic
  source 1..87: mol_type = other DNA
aagcttgaggactcggccgtgtatctctgtgccagcagcgaccagctacgacggactacaagaaactacgagcagtacttcgggccg

SEQ ID NO: 84 - DNA, 87 bp, synthetic construct
  misc_feature 1..87: note = Synthetic
  source 1..87: mol_type = other DNA
cagtccacggagtcaggggacacagcactgtatttctgtgccagcagcaaagcgctttgggttacgggtggctacaccttcggttcg

SEQ ID NO: 85 - DNA, 87 bp, synthetic construct
  misc_feature 1..87: note = Synthetic
  source 1..87: mol_type = other DNA
cacgccctgcagccagaagactcagccctgtatctctgcgccagcagccaagacagggtgaacaccggggagctgttttttggagaa

SEQ ID NO: 86 - DNA, 87 bp, synthetic construct
  misc_feature 1..87: note = Synthetic
  source 1..87: mol_type = other DNA
acagtgaccagtgcccatcctgaagacagcagcttctacatctgcagtgctagacggggcgataattcacccctccactttgggaat

SEQ ID NO: 87 - DNA, 87 bp, synthetic construct
  misc_feature 1..87: note = Synthetic
  variation 1: note = n is a, c, g, or t
  source 1..87: mol_type = other DNA
nctctcactgtgacatcggcccaaaagaacccgacagctttctatctctgtgccagtcagactcacaatgagcagttcttcgggcca

SEQ ID NO: 88 - DNA, 87 bp, synthetic construct
  misc_feature 1..87: note = Synthetic
  source 1..87: mol_type = other DNA
gaactaaacctgagctctctggagctgggggactcagctttgtatttctgtgccagcagccctcaaactgaagctttctttggacaa

SEQ ID NO: 89 - DNA, 87 bp, synthetic construct
  misc_feature 1..87: note = Synthetic
  source 1..87: mol_type = other DNA
cagcgcacacagcaggaggactccgccgtgtatctctgtgccagcagctcatttcggggggccggggagacccagtacttcgggcca

SEQ ID NO: 90 - DNA, 87 bp, synthetic construct
  misc_feature 1..87: note = Synthetic
  source 1..87: mol_type = other DNA
gtgaccagtgcccatcctgaagacagcagcttctacatctgcagtgctagaaacccgctcgctacctacgagcagtacttcgggccg

SEQ ID NO: 91 - DNA, 87 bp, synthetic construct
  misc_feature 1..87: note = Synthetic
  source 1..87: mol_type = other DNA
gagtctgccatccccaaccagacagctctttacttctgtgccaccagtcaggggacacgacctctctcagcccagcattttggtgat

SEQ ID NO: 92 - DNA, 87 bp, synthetic construct
  misc_feature 1..87: note = Synthetic
  source 1..87: mol_type = other DNA
gctgctccctcccagacatctgtgtacttctgtgccagcagtcacaaagggacaggggtccttgattcacccctccactttgggaat

SEQ ID NO: 93 - DNA, 87 bp, synthetic construct
  misc_feature 1..87: note = Synthetic
  source 1..87: mol_type = other DNA
aatgtgaacgccttgttgctgggggactcggccctctatctctgtgccagcagctttacaggtggcgatgagcagttcttcgggcca

SEQ ID NO: 94 - DNA, 87 bp, synthetic construct
  misc_feature 1..87: note = Synthetic
  source 1..87: mol_type = other DNA
gccttgttgctgggggactcggccctctatctctgtgccagcagcttagggctagcgggaggtcgaaacattcagtacttcggcgcc

SEQ ID NO: 95 - DNA, 87 bp, synthetic construct
  misc_feature 1..87: note = Synthetic
  source 1..87: mol_type = other DNA
agtgcccatcctgaagacagcagcttctacatctgcagtgccccagccagaggcgccgaaactttctacgagcagtacttcgggccg

SEQ ID NO: 96 - DNA, 87 bp, synthetic construct
  misc_feature 1..87: note = Synthetic
  source 1..87: mol_type = other DNA
gtgagcaccttggagctgggggactcggccctttatctttgcgccagcatgggggatactggggccaacgtcctgactttcggggcc

SEQ ID NO: 97 - DNA, 87 bp, synthetic construct
  misc_feature 1..87: note = Synthetic
  source 1..87: mol_type = other DNA
ttggagctgggggactcggccctttatctttgcgccagcagccctccccgtgggggagggaacaccggggagctgttttttggagaa

SEQ ID NO: 98 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccaaacatctgtgtacttctgtgccagccggagggacttaaactatggctacaccttcggttcg

SEQ ID NO: 99 - DNA, 87 bp, synthetic construct
  misc_feature 1..87: note = Synthetic
  source 1..87: mol_type = other DNA
atcaattccctggagcttggtgactctgctgtgtatttctgtgccagcagccaacgagtacggaattcacccctccactttgggaat

SEQ ID NO: 100 - DNA, 87 bp, synthetic construct
  misc_feature 1..87: note = Synthetic
  source 1..87: mol_type = other DNA
agctctctggagctgggggactcagctttgtatttctgtgccagcaccgccacagggcactataattcacccctccactttgggaat

SEQ ID NO: 101 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccagcagtgagttacgtaacaatgagcagttcttcgggcca

SEQ ID NO: 102 - DNA, 87 bp, synthetic construct
  misc_feature 1..87: note = Synthetic
  source 1..87: mol_type = other DNA
acagagcagggggactcggccatgtatctctgtgccagcagcgtcgttaggacggacagggggcgggagacccagtacttcgggcca

SEQ ID NO: 103 - DNA, 87 bp, synthetic construct
  misc_feature 1..87: note = Synthetic
  source 1..87: mol_type = other DNA
accagtgcccatcctgaagacagcagcttctacatctgcagtgcccgatttgtacggggacgcaatcagccccagcattttggtgat

SEQ ID NO: 104 - DNA, 87 bp, synthetic construct
  misc_feature 1..87: note = Synthetic
  source 1..87: mol_type = other DNA
gtgagcaccttggagctgggggactcggccctttatctttgcgccagcgggacagccccggacagaaatgagcagttcttcgggcca

SEQ ID NO: 105 - DNA, 87 bp, synthetic construct
  misc_feature 1..87: note = Synthetic
  source 1..87: mol_type = other DNA
actctggagtcagctacccgctcccagacatctgtgtatttctgcgccagcagagcagggggagatgagacccagtacttcgggcca

SEQ ID NO: 106 - DNA, 87 bp, synthetic construct
  misc_feature 1..87: note = Synthetic
  source 1..87: mol_type = other DNA
ttgttgctgggggactcggccctctatctctgtgccagcagctttgtaaccagcgggagagtaacagatacgcagtattttggccca

SEQ ID NO: 107 - DNA, 87 bp, synthetic construct
  misc_feature 1..87: note = Synthetic
  source 1..87: mol_type = other DNA
atccagcctgcaaagcttgaggactcggccgtgtatctctgtgccagcagcttagaaagggggtggaatgagcagttcttcgggcca

SEQ ID NO: 108 - DNA, 87 bp, synthetic construct
  misc_feature 1..87: note = Synthetic
  source 1..87: mol_type = other DNA
cttgaggactcggccgtgtatctctgtgccagcagctccccgagactagcgggagggccttcagcctacgagcagtacttcgggccg

SEQ ID NO: 109 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccagcagtgaaagggagaatcagccccagcattttggtgat

SEQ ID NO: 110 - DNA, 87 bp, synthetic construct
  misc_feature 1..87: note = Synthetic
  source 1..87: mol_type = other DNA
aagatccggtccacaaagctggaggactcagccatgtacttctgtgccagcagtgtgagacagtcctacgagcagtacttcgggccg

SEQ ID NO: 111 - DNA, 87 bp, synthetic construct
  misc_feature 1..87: note = Synthetic
  source 1..87: mol_type = other DNA
gagtctgccatccccaaccagacagctctttacttctgtgccaccaggcctggacagagtttgaacactgaagctttctttggacaa

SEQ ID NO: 112 - DNA, 87 bp, synthetic construct
  misc_feature 1..87: note = Synthetic
  source 1..87: mol_type = other DNA
ctggagtccgccagcaccaaccagacatctatgtacctctgtgccagcagtttatgggggggcgatcagccccagcattttggtgat

SEQ ID NO: 113 - DNA, 87 bp, synthetic construct
  misc_feature 1..87: note = Synthetic
  source 1..87: mol_type = other DNA
ctgcagccagaagactcggccctgtatctctgcgccagcagccacagaggcagcgggagagccccagatacgcagtattttggccca

SEQ ID NO: 114 - DNA, 87 bp, synthetic construct
  misc_feature 1..87: note = Synthetic
  variation 1: note = n is a, c, g, or t
  source 1..87: mol_type = other DNA
ntgtcggctgctccctcccagacatctgtgtacttctgtgccagcaggggactagcggggagcggcggggagctgttttttggagaa

SEQ ID NO: 115 - DNA, 87 bp, synthetic construct
  misc_feature 1..87: note = Synthetic
  source 1..87: mol_type = other DNA
gcccaaaagaacccgacagctttctatctctgtgccagtagtatagattcgacagggaaggggccagatacgcagtattttggccca

SEQ ID NO: 116 - DNA, 87 bp, synthetic construct
  misc_feature 1..87: note = Synthetic
  source 1..87: mol_type = other DNA
aggatccagcaggtagtgcgaggagattcggcagcttatttctgtgccagctcaccaccgagtcgatatggctacaccttcggttcg

SEQ ID NO: 117 - DNA, 87 bp, synthetic construct
  misc_feature 1..87: note = Synthetic
  source 1..87: mol_type = other DNA
tcaccaggcctgggggacgcagccatgtacctgtgtgccaccagcagcagctacaggggtaggagtcagacccagtacttcgggcca

SEQ ID NO: 118 - DNA, 87 bp, synthetic construct
  misc_feature 1..87: note = Synthetic
  source 1..87: mol_type = other DNA
ctggagtcgcccagccccaaccagacctctctgtacttctgtgccagcagtctagcggggctaggcaatgagcagttcttcgggcca

SEQ ID NO: 119 - DNA, 87 bp, synthetic construct
  misc_feature 1..87: note = Synthetic
  source 1..87: mol_type = other DNA
tccctggagcttggtgactctgctgtgtatttctgtgccagcagccaagatgggctagcggggtttcttgagcagttcttcgggcca

SEQ ID NO: 120 - DNA, 87 bp, synthetic construct
  misc_feature 1..87: note = Synthetic
  source 1..87: mol_type = other DNA
acagtgaccagtgcccatcctgaagacagcagcttctacatctgcagtgcgggcgggagagttgcggagacccagtacttcgggcca

SEQ ID NO: 121 - DNA, 87 bp, synthetic construct
  misc_feature 1..87: note = Synthetic
  source 1..87: mol_type = other DNA
ttggcgtctgctgtaccctctcagacatctgtgtacttctgtgccagcagtgaacagggagtttaccatgagcagttcttcgggcca

SEQ ID NO: 122 - DNA, 87 bp, synthetic construct
  misc_feature 1..87: note = Synthetic
  source 1..87: mol_type = other DNA
aagctccttctcagtgactctggcttctatctctgtgcctggagtaccccgggactacgtagcgttggtacgcagtattttggccca

SEQ ID NO: 123 - DNA, 87 bp, synthetic construct
  misc_feature 1..87: note = Synthetic
  source 1..87: mol_type = other DNA
gagtcgcccagccccaaccagacctctctgtacttctgtgccagcagttccccgacagggagggacactgaagctttctttggacaa

SEQ ID NO: 124 - DNA, 87 bp, synthetic construct
  misc_feature 1..87: note = Synthetic
  source 1..87: mol_type = other DNA
tctaagaagctccttctcagtgactctggcttctatctctgtgcctggagtgtacagggcagggggaatggctacaccttcggttcg

SEQ ID NO: 125 - DNA, 87 bp, synthetic construct
  misc_feature 1..87: note = Synthetic
  source 1..87: mol_type = other DNA
ctgacagtgaccagtgcccatcctgaagacagcagcttctacatctgcagtccgactagcggggaagagacccagtacttcgggcca

SEQ ID NO: 126 - DNA, 87 bp, synthetic construct
  misc_feature 1..87: note = Synthetic
  source 1..87: mol_type = other DNA
atccagccctcagaacccagggactcagctgtgtacttctgtgccagcagtttgagacagggagcccacgagcagtacttcgggccg

SEQ ID NO: 127 - DNA, 87 bp, synthetic construct
  misc_feature 1..87: note = Synthetic
  source 1..87: mol_type = other DNA
tcgcccagccccaaccagacctctctgtacttctgtgccagcggggcccggactagcgggagacgggcggtgctgttttttggagaa

SEQ ID NO: 128 - DNA, 87 bp, synthetic construct
  misc_feature 1..87: note = Synthetic
  source 1..87: mol_type = other DNA
accttggagctgggggactcggccctttatctttgcgccagcagcttggtactagcgggccgaaacaatgagcagttcttcgggcca

SEQ ID NO: 129 - DNA, 87 bp, synthetic construct
  misc_feature 1..87: note = Synthetic
  source 1..87: mol_type = other DNA
tctaagaagctccttctcagtgactctggcttctatctctgtgcctgggaagtgaagggacccaatcagccccagcattttggtgat

SEQ ID NO: 130 - DNA, 87 bp, synthetic construct
  misc_feature 1..87: note = Synthetic
  source 1..87: mol_type = other DNA
tcctcagaaccgggagacacggcactgtatctctgcgccagcagtcaatccaggcaggaggcttcctacgagcagtacttcgggccg

SEQ ID NO: 131 - DNA, 87 bp, synthetic construct
  misc_feature 1..87: note = Synthetic
  source 1..87: mol_type = other DNA
ccagaagactcggccctgtatctctgcgccagcagccaaggcgggactagcgggagggccctccgagatacgcagtattttggccca

SEQ ID NO: 132 - DNA, 87 bp, synthetic construct
  misc_feature 1..87: note = Synthetic
  source 1..87: mol_type = other DNA
cagcgcacagagcagggggactcggccatgtatctctgtgccagcagcccccagaggggattgaacactgaagctttctttggacaa

SEQ ID NO: 133 - DNA, 87 bp, synthetic construct
  misc_feature 1..87: note = Synthetic
  source 1..87: mol_type = other DNA
gagcaggaggactcggccgtgtatctctgtgccagcagccccgccgggggagttgcggccagctcctacgagcagtacttcgggccg

SEQ ID NO: 134 - DNA, 87 bp, synthetic construct
  misc_feature 1..87: note = Synthetic
  source 1..87: mol_type = other DNA
acagagcagggggactcggccatgtatctctgtgccagcagcttaggtaattggcggaccccgagctatggctacaccttcggttcg

SEQ ID NO: 135 - DNA, 87 bp, synthetic construct
  misc_feature 1..87: note = Synthetic
  source 1..87: mol_type = other DNA
ctggcaatcctgtcctcagaaccgggagacacggcactgtatctctgcgccagcagtcacccgaactatggctacaccttcggttcg

SEQ ID NO: 136 - DNA, 87 bp, synthetic construct
  misc_feature 1..87: note = Synthetic
  variation 1..7: note = n is a, c, g, or t
  source 1..87: mol_type = other DNA
nnnnnnntgtcggctgctccctcccagacatctgtgtacttctgtgccagcagtcggacaggcaatcagccccagcattttggtgat

SEQ ID NO: 137 - DNA, 87 bp, synthetic construct
  misc_feature 1..87: note = Synthetic
  source 1..87: mol_type = other DNA
gtgaccagtgcccatcctgaagacagcagcttctacatctgcagtgctagagaaggggggttcgggaacgagcagtacttcgggccg

SEQ ID NO: 138 - DNA, 87 bp, synthetic construct
  misc_feature 1..87: note = Synthetic
  source 1..87: mol_type = other DNA
aggatccagcaggtagtgcgaggagattcggcagcttatttctgtgccagcgcccaggggggggacactgaagctttctttggacaa

SEQ ID NO: 139 - DNA, 87 bp, synthetic construct
  misc_feature 1..87: note = Synthetic
  source 1..87: mol_type = other DNA
actgtgacatcggcccaaaagaacccgacagctttctatctctgtgccagtagtccattacagaccggggagctgttttttggagaa

SEQ ID NO: 140 - DNA, 87 bp, synthetic construct
  misc_feature 1..87: note = Synthetic
  source 1..87: mol_type = other DNA
actctgacagtgaccagtgcccatcctgaagacagcagcttctacatctgcagtgccccaggagggtatggctacaccttcggttcg

SEQ ID NO: 141 - DNA, 87 bp, synthetic construct
  misc_feature 1..87: note = Synthetic
  source 1..87: mol_type = other DNA
acagtgaccagtgcccatcctgaagacagcagcttctacatctgcagtgcaaaagacagggggaagaggggctacaccttcggttcg

SEQ ID NO: 142 - DNA, 87 bp, synthetic construct
  misc_feature 1..87: note = Synthetic
  source 1..87: mol_type = other DNA
tctaagaagctccttctcagtgactctggcttctatctctgtgcctggacctaccggacgggaggagtcgagcagtacttcgggccg

SEQ ID NO: 143 - DNA, 87 bp, synthetic construct
  misc_feature 1..87: note = Synthetic
  source 1..87: mol_type = other DNA
agcaccttggagctgggggactcggccctttatctttgcgccagcagcttggacagcaaagccaaaaacattcagtacttcggcgcc

SEQ ID NO: 144 - DNA, 87 bp, synthetic construct
  misc_feature 1..87: note = Synthetic
  source 1..87: mol_type = other DNA
tcagaacccagggactcagctgtgtatttttgtgctagtggtttgaggactagcgggaagttgcgtaatgagcagttcttcgggcca

SEQ ID NO: 145 - DNA, 87 bp, synthetic construct
  misc_feature 1..87: note = Synthetic
  source 1..87: mol_type = other DNA
atccagcgcacagagcagggggactcggccatgtatctctgtgccagcagcttaggggctagcgggactgagcagttcttcgggcca

SEQ ID NO: 146 - DNA, 87 bp, synthetic construct
  misc_feature 1..87: note = Synthetic
  source 1..87: mol_type = other DNA
agcaccttggagctgggggactcggccctttatctttgcgccagcagctcggacagggggctacacgaaaaactgttttttggcagt

SEQ ID NO: 147 - DNA, 87 bp, synthetic construct
  misc_feature 1..87: note = Synthetic
  source 1..87: mol_type = other DNA
gctgctccctcccagacatctgtgtacttctgtgccagcagcctacgcggcggggggaatggttgggatacgcagtattttggccca

SEQ ID NO: 148 - DNA, 87 bp, synthetic construct
  misc_feature 1..87: note = Synthetic
  source 1..87: mol_type = other DNA
ctagagtctgccatccccaaccagacagctctttacttctgtgccaccagaggggggccggatgaagagacccagtacttcgggcca

SEQ ID NO: 149 - DNA, 87 bp, synthetic construct
  misc_feature 1..87: note = Synthetic
  source 1..87: mol_type = other DNA
aacgccttggagctggacgactcggccctgtatctctgtgccagcagctccccgacagggggtgactacgagcagtacttcgggccg

SEQ ID NO: 150 - DNA, 87 bp, synthetic construct
  misc_feature 1..87: note = Synthetic
  source 1..87: mol_type = other DNA
gtgaacgccttgttgctgggggactcggccctgtatctctgtgccagcagcttgtccggaagtaatgaaaaactgttttttggcagt

SEQ ID NO: 151 - DNA, 87 bp, synthetic construct
  misc_feature 1..87: note = Synthetic
  source 1..87: mol_type = other DNA
cctgcaaagcttgaggactcggccgtgtatctctgtgccagcagcttgaggcgtccgggacaggggtgggagctgttttttggagaa

SEQ ID NO: 152 - DNA, 87 bp, synthetic construct
  misc_feature 1..87: note = Synthetic
  source 1..87: mol_type = other DNA
gagtccgccagcaccaaccagacatctatgtacctctgtgccagcagtctcgcgacagggggcttatcacccctccactttgggaat

SEQ ID NO: 153 - DNA, 87 bp, synthetic construct
  misc_feature 1..87: note = Synthetic
  variation 1..4: note = n is a, c, g, or t
  source 1..87: mol_type = other DNA
nnnnctctgaagatccggtccacaaagctggaggactcagccatgtacttctgtgccagcagggatgatggctacaccttcggttcg

SEQ ID NO: 154 - DNA, 87 bp, synthetic construct
  misc_feature 1..87: note = Synthetic
  source 1..87: mol_type = other DNA
agccccaaccagacctctctgtacttctgtgccagcagttcccccggactagcgggagggaacaccggggagctgttttttggagaa

SEQ ID NO: 155 - DNA, 87 bp, synthetic construct
  misc_feature 1..87: note = Synthetic
  source 1..87: mol_type = other DNA
gctccctcccaaacatctgtgtacttctgtgccagcagttactccccggcgatcgggggggtctacaatgagcagttcttcgggcca

SEQ ID NO: 156 - DNA, 87 bp, synthetic construct
  misc_feature 1..87: note = Synthetic
  source 1..87: mol_type = other DNA
agtgcccatcctgaagacagcagcttctacatctgcagtggaaagggactagcgggaggggctcaagagacccagtacttcgggcca

SEQ ID NO: 157 - DNA, 87 bp, synthetic construct
  misc_feature 1..87: note = Synthetic
  source 1..87: mol_type = other DNA
gagatccagcgcacagagcagggggactcggccatgtatctctgtgccagcacccaggggggggtagagacccagtacttcgggcca

SEQ ID NO: 158 - DNA, 87 bp, synthetic construct
  misc_feature 1..87: note = Synthetic
  source 1..87: mol_type = other DNA
cacaccctgcagccagaagactcggccctgtatctctgcgccagcagccaagagcagggtcgcaattcacccctccactttgggaat

SEQ ID NO: 159 - DNA, 87 bp, synthetic construct
  misc_feature 1..87: note = Synthetic
  source 1..87: mol_type = other DNA
aatgtgagcaccttggagctgggggactcggccctttatctttgcgccagcagccagtacaatcacaatgagcagttcttcgggcca

SEQ ID NO: 160 - DNA, 87 bp, synthetic construct
  misc_feature 1..87: note = Synthetic
  source 1..87: mol_type = other DNA
atccagccctcagaacccagggactcagctgtgtatttttgtgctagtggtttggcccccctgagccgggagcagtacttcgggccg

SEQ ID NO: 161 - DNA, 87 bp, synthetic construct
  misc_feature 1..87: note = Synthetic
  source 1..87: mol_type = other DNA
cagccctcagaacccagggactcagctgtgtacttctgtgccagcagtttaggggggtcgagcaatcagccccagcattttggtgat

SEQ ID NO: 162 - DNA, 87 bp, synthetic construct
  misc_feature 1..87: note = Synthetic
  source 1..87: mol_type = other DNA
cagcaggtagtgcgaggagattcggcagcttatttctgtgccagctcaccctggccccaggatggtgaaaaactgttttttggcagt

SEQ ID NO: 163 - DNA, 87 bp, synthetic construct
  misc_feature 1..87: note = Synthetic
  source 1..87: mol_type = other DNA
accagtgcccatcctgaagacagcagcttctacatctgcagtgctagaaaggtgcaacggggcaaaaacattcagtacttcggcgcc

SEQ ID NO: 164 - DNA, 87 bp, synthetic construct
  misc_feature 1..87: note = Synthetic
  source 1..87: mol_type = other DNA
gtgaccagtgcccatcctgaagacagcagcttctacatctgcagtgctagagcggatggacagggctatggctacaccttcggttcg

SEQ ID NO: 165 - DNA, 87 bp, synthetic construct
  misc_feature 1..87: note = Synthetic
  source 1..87: mol_type = other DNA
cacaccctgcagccagaagactcggccctgtatctctgcgccagcagccaagaggtagggggagaccatggctacaccttcggttcg

SEQ ID NO: 166 - DNA, 87 bp, synthetic construct
  misc_feature 1..87: note = Synthetic
  variation 1..4: note = n is a, c, g, or t
  source 1..87: mol_type = other DNA
nnnntgtcggctgctccctcccagacatctgtgtacttctgtgccagcatccgccccggtcttaactatggctacaccttcggttcg

SEQ ID NO: 167 - DNA, 87 bp, synthetic construct
  misc_feature 1..87: note = Synthetic
  source 1..87: mol_type = other DNA
atccagcaggtagtgcgaggagattcggcagcttatttctgtgccagctcaccagctgacagggaagagacccagtacttcgggcca

SEQ ID NO: 168 - DNA, 87 bp, synthetic construct
  misc_feature 1..87: note = Synthetic
  source 1..87: mol_type = other DNA
gagtctgccatccccaaccagacagctctttacttctgtgccaccagtgaaccgggacccttaaatcagccccagcattttggtgat

SEQ ID NO: 169 - DNA, 87 bp, synthetic construct
  misc_feature 1..87: note = Synthetic
  source 1..87: mol_type = other DNA
gagtcgcccagccccaaccagacctctctgtacttctgtgccagcagaaccacgacagggttcctttcacccctccactttgggaac

SEQ ID NO: 170 - DNA, 87 bp, synthetic construct
  misc_feature 1..87: note = Synthetic
  source 1..87: mol_type = other DNA
acagtgaccagtgcccatcctgaagacagcagcttctacatctgcagtggattagcgggaggccaggagacccagtacttcgggcca

SEQ ID NO: 171 - DNA, 87 bp, synthetic construct
  misc_feature 1..87: note = Synthetic
  variation 1: note = n is a, c, g, or t
  source 1..87: mol_type = other DNA
ntgtcggctgctccctcccagacatctgtgtacttctgtgccagcagcaaaccgggacagggctgggagacccagtacttcgggcca

SEQ ID NO: 172 - DNA, 87 bp, synthetic construct
  misc_feature 1..87: note = Synthetic
  source 1..87: mol_type = other DNA
atccgctcaccaggcctgggggacgcagccatgtacctgtgtgccaccagcagcagcgggttctcatacgagcagtacttcgggccg

SEQ ID NO: 173 - DNA, 87 bp, synthetic construct
  misc_feature 1..87: note = Synthetic
  source 1..87: mol_type = other DNA
gctgctccctcccagacatctgtgtacttctgtgccagcagttacattgtgggattgacaactaatgaaaaactgttttttggcagt

SEQ ID NO: 174 - DNA, 87 bp, synthetic construct
  misc_feature 1..87: note = Synthetic
  source 1..87: mol_type = other DNA
agttctaagaagctccttctcagtgactctggcttctatctctgtgcctgggggactctctggagcggggagctgttttttggagaa

SEQ ID NO: 175 - DNA, 87 bp, synthetic construct
  misc_feature 1..87: note = Synthetic
  source 1..87: mol_type = other DNA
tccgccagcaccaaccagacatctatgtacctctgtgccagcccccctgttagcgggacagactacaatgagcagttcttcgggcca

SEQ ID NO: 176 - DNA, 87 bp, synthetic construct
  misc_feature 1..87: note = Synthetic
  source 1..87: mol_type = other DNA
aacgccttggagctggacgactcggccctgtatctctgtgccagcagcttatacgacaggcccagatatggctacaccttcggttcg

SEQ ID NO: 177 - DNA, 87 bp, synthetic construct
  misc_feature 1..87: note = Synthetic
  source 1..87: mol_type = other DNA
attctggagtccgccagcaccaaccagacatctatgtacctctgtgccagctacggacagggggtgcatgagcagttcttcgggcca

SEQ ID NO: 178 - DNA, 87 bp, synthetic construct
  misc_feature 1..87: note = Synthetic
  source 1..87: mol_type = other DNA
gtagtgcgaggagattcggcagcttatttctgtgccagctcaccaccgatcggattcacgactaatgaaaaactgttttttggcagt

SEQ ID NO: 179 - protein, 15 aa, synthetic construct
  REGION 1..15: note = Synthetic
CASSIPQGSGSPLHF

SEQ ID NO: 180 - protein, 15 aa, synthetic construct
  REGION 1..15: note = Synthetic
CASSYGGGPPDTQYF

SEQ ID NO: 181 - protein, 16 aa, synthetic construct
  REGION 1..16: note = Synthetic
CASSQTALYSGNTIYF

SEQ ID NO: 182 - protein, 13 aa, synthetic construct
  REGION 1..13: note = Synthetic
CATALAGGHEQYF

SEQ ID NO: 183 - protein, 15 aa, synthetic construct
  REGION 1..15: note = Synthetic
CSAREGALDNSPLHF

SEQ ID NO: 184 - protein, 16 aa, synthetic construct
  REGION 1..16: note = Synthetic
CATSDFGKEGDEKLFF

SEQ ID NO: 185 - protein, 12 aa, synthetic construct
  REGION 1..12: note = Synthetic
CASSFTYDEQFF

SEQ ID NO: 186 - protein, 15 aa, synthetic construct
  REGION 1..15: note = Synthetic
CASSPGREPGNTIYF

SEQ ID NO: 187 - protein, 13 aa, synthetic construct
  REGION 1..13: note = Synthetic
CASTATESSPLHF

SEQ ID NO: 188 - protein, 14 aa, synthetic construct
  REGION 1..14: note = Synthetic
CASRKTGSLYGYTF

SEQ ID NO: 189 - protein, 15 aa, synthetic construct
  REGION 1..15: note = Synthetic
CASSLRTGIINEQFF

SEQ ID NO: 190 - protein, 16 aa, synthetic construct
  REGION 1..16: note = Synthetic
CASSTTGLAAYNEQFF

SEQ ID NO: 191 - protein, 16 aa, synthetic construct
  REGION 1..16: note = Synthetic
CASSFVDSPESYEQYF

SEQ ID NO: 192 - protein, 13 aa, synthetic construct
  REGION 1..13: note = Synthetic
CASSLTRIPEQYF

SEQ ID NO: 193 - protein, 17 aa, synthetic construct
  REGION 1..17: note = Synthetic
CSARVTGGGAHTGELFF

SEQ ID NO: 194 - protein, 16 aa, synthetic construct
  REGION 1..16: note = Synthetic
CASSLALGTGDTEAFF

SEQ ID NO: 195 - protein, 15 aa, synthetic construct
  REGION 1..15: note = Synthetic
CASSLAGTGTNEQFF

SEQ ID NO: 196 - protein, 14 aa, synthetic construct
  REGION 1..14: note = Synthetic
CASRGQRDTGELFF

SEQ ID NO: 197 - protein, 13 aa, synthetic construct
  REGION 1..13: note = Synthetic
CASSIRPNDEQFF

SEQ ID NO: 198 - protein, 15 aa, synthetic construct
  REGION 1..15: note = Synthetic
CASSYSSGGGNEQFF

SEQ ID NO: 199 - protein, 17 aa, synthetic construct
  REGION 1..17: note = Synthetic
CASRTIGQGSFNYGYTF

SEQ ID NO: 200 - protein, 15 aa, synthetic construct
  REGION 1..15: note = Synthetic
CASSSLTPEAYGYTF

SEQ ID NO: 201 - protein, 14 aa, synthetic construct
  REGION 1..14: note = Synthetic
CSAMSPSGTGELFF

SEQ ID NO: 202 - protein, 14 aa, synthetic construct
  REGION 1..14: note = Synthetic
CASSDDLAANVLTF

SEQ ID NO: 203 - protein, 16 aa, synthetic construct
  REGION 1..16: note = Synthetic
CASSPRAGGAVREQYF

SEQ ID NO: 204 - protein, 16 aa, synthetic construct
  REGION 1..16: note = Synthetic
CAWSVGGPGANSPLHF

SEQ ID NO: 205 - protein, 12 aa, synthetic construct
  REGION 1..12: note = Synthetic
CASSKVNEKLFF

SEQ ID NO: 206 - protein, 15 aa, synthetic construct
  REGION 1..15: note = Synthetic
CASSLGGRSNQPQHF

SEQ ID NO: 207 - protein, 16 aa, synthetic construct
  REGION 1..16: note = Synthetic
CSAREPHDTSTDTQYF

SEQ ID NO: 208 - protein, 14 aa, synthetic construct
  REGION 1..14: note = Synthetic
CSASRTYDNQPQHF

SEQ ID NO: 209 - protein, 17 aa, synthetic construct
  REGION 1..17: note = Synthetic
CASSWDRTKFGNQPQHF

SEQ ID NO: 210 - protein, 15 aa, synthetic construct
  REGION 1..15: note = Synthetic
CASSSSGSNYNEQFF

SEQ ID NO: 211 - protein, 11 aa, synthetic construct
  REGION 1..11: note = Synthetic
CSARWYNEQFF

SEQ ID NO: 212 - protein, 15 aa, synthetic construct
  REGION 1..15: note = Synthetic
CASSPLLYSGNTIYF

SEQ ID NO: 213 - protein, 15 aa, synthetic construct
  REGION 1..15: note = Synthetic
CASSLPRDRDYGYTF

SEQ ID NO: 214 - protein, 15 aa, synthetic construct
  REGION 1..15: note = Synthetic
CASSELGLNTDTQYF

SEQ ID NO: 215 - protein, 13 aa, synthetic construct
  REGION 1..13: note = Synthetic
CASSAMVEQPQHF

SEQ ID NO: 216 - protein, 15 aa, synthetic construct
  REGION 1..15: note = Synthetic
CASSEMGLNTDTQYF

SEQ ID NO: 217 - protein, 15 aa, synthetic construct
  REGION 1..15: note = Synthetic
CASSPRGAVNTEAFF

SEQ ID NO: 218 - protein, 15 aa, synthetic construct
  REGION 1..15: note = Synthetic
CASSELGTRPNEQFF

SEQ ID NO: 219 - protein, 15 aa, synthetic construct
  REGION 1..15: note = Synthetic
CASRKQGLGYTEAFF

SEQ ID NO: 220 - protein, 14 aa, synthetic construct
  REGION 1..14: note = Synthetic
CASSYRQTNQPQHF

SEQ ID NO: 221 - protein, 10 aa, synthetic construct
  REGION 1..10: note = Synthetic
CSVLQHEQYF

SEQ ID NO: 222 - protein, 14 aa, synthetic construct
  REGION 1..14: note = Synthetic
CASSPQPSTDTQYF

SEQ ID NO: 223 - protein, 13 aa, synthetic construct
  REGION 1..13: note = Synthetic
CASSVRDYSPLHF

SEQ ID NO: 224 - protein, 14 aa, synthetic construct
  REGION 1..14: note = Synthetic
CASSLERGAYGYTF

SEQ ID NO: 225 - protein, 16 aa, synthetic construct
  REGION 1..16: note = Synthetic
CATSQGVGSGANVLTF

SEQ ID NO: 226 - protein, 13 aa, synthetic construct
  REGION 1..13: note = Synthetic
CSATGGTNEKLFF

SEQ ID NO: 227 - protein, 14 aa, synthetic construct
  REGION 1..14: note = Synthetic
CASRETGGAGELFF

SEQ ID NO: 228 - protein, 15 aa, synthetic construct
  REGION 1..15: note = Synthetic
CASSISLALIYEQYF

SEQ ID NO: 229 - protein, 15 aa, synthetic construct
  REGION 1..15: note = Synthetic
CASSLYAGQNTEAFF

SEQ ID NO: 230 - protein, 15 aa, synthetic construct
  REGION 1..15: note = Synthetic
CASSPDFSGANVLTF

SEQ ID NO: 231 - protein, 15 aa, synthetic construct
  REGION 1..15: note = Synthetic
CASSQTGQGYNEQFF

SEQ ID NO: 232 - protein, 17 aa, synthetic construct
  REGION 1..17: note = Synthetic
CATSEGGRGRGPGELFF

SEQ ID NO: 233 - protein, 15 aa, synthetic construct
  REGION 1..15: note = Synthetic
CASSLDGSSGNTIYF

SEQ ID NO: 234 - protein, 15 aa, synthetic construct
  REGION 1..15: note = Synthetic
CASSFPSRGNTEAFF

SEQ ID NO: 235 - protein, 14 aa, synthetic construct
  REGION 1..14: note = Synthetic
CASTSGERGPEAFF

SEQ ID NO: 236 - protein, 15 aa, synthetic construct
  REGION 1..15: note = Synthetic
CASSRGTANTGELFF

SEQ ID NO: 237 - protein, 13 aa, synthetic construct
  REGION 1..13: note = Synthetic
CASLSGHQETQYF

SEQ ID NO: 238 - protein, 18 aa, synthetic construct
  REGION 1..18: note = Synthetic
CSARNRMGGTSSYNEQFF

SEQ ID NO: 239 - protein, 17 aa, synthetic construct
  REGION 1..17: note = Synthetic
CASSSSRLAGVTDTQYF

SEQ ID NO: 240 - protein, 15 aa, synthetic construct
  REGION 1..15: note = Synthetic
CASSLSRGVYYEQYF

SEQ ID NO: 241 - protein, 17 aa, synthetic construct
  REGION 1..17: note = Synthetic
CASSQTGGRGQNSPLHF

SEQ ID NO: 242 - protein, 14 aa, synthetic construct
  REGION 1..14: note = Synthetic
CASSVGGGPYEQYF

SEQ ID NO: 243 - protein, 13 aa, synthetic construct
  REGION 1..13: note = Synthetic
CASSRLAGYEQYF

SEQ ID NO: 244 - protein, 14 aa, synthetic construct
  REGION 1..14: note = Synthetic
CASRLAGAGDTQYF

SEQ ID NO: 245 - protein, 17 aa, synthetic construct
  REGION 1..17: note = Synthetic
CASSPLSGLALYNEQFF

SEQ ID NO: 246 - protein, 16 aa, synthetic construct
  REGION 1..16: note = Synthetic
CASRLLGGEDSTEAFF

SEQ ID NO: 247 - protein, 14 aa, synthetic construct
  REGION 1..14: note = Synthetic
CSAVYQGVNTEAFF

SEQ ID NO: 248 - protein, 13 aa, synthetic construct
  REGION 1..13: note = Synthetic
CASSEGGNQPQHF

SEQ ID NO: 249 - protein, 13 aa, synthetic construct
  REGION 1..13: note = Synthetic
CAISEGAGEKLFF

SEQ ID NO: 250 - protein, 12 aa, synthetic construct
  REGION 1..12: note = Synthetic
CASRGGGTEAFF

SEQ ID NO: 251 - protein, 13 aa, synthetic construct
  REGION 1..13: note = Synthetic
CSARGDSSGELFF

SEQ ID NO: 252 - protein, 17 aa, synthetic construct
  REGION 1..17: note = Synthetic
CAISDRRTSESTDTQYF

SEQ ID NO: 253 - protein, 13 aa, synthetic construct
  REGION 1..13: note = Synthetic
CASRDLGGVAIYF

SEQ ID NO: 254 - protein, 11 aa, synthetic construct
  REGION 1..11: note = Synthetic
CSAKRHHEQYF

SEQ ID NO: 255 - protein, 12 aa, synthetic construct
  REGION 1..12: note = Synthetic
CASSRPASEQFF

SEQ ID NO: 256 - protein, 13 aa, synthetic construct
  REGION 1..13: note = Synthetic
CASSGVNNTEAFF

SEQ ID NO: 257 - protein, 14 aa, synthetic construct
  REGION 1..14: note = Synthetic
CSVEFRDRDTEAFF

SEQ ID NO: 258 - protein, 12 aa, synthetic construct
  REGION 1..12: note = Synthetic
CSARSSDTEAFF

SEQ ID NO: 259 - protein, 13 aa, synthetic construct
  REGION 1..13: note = Synthetic
CASVTSGIHEQFF

SEQ ID NO: 260 - protein, 13 aa, synthetic construct
  REGION 1..13: note = Synthetic
CSASKQSNYGYTF

SEQ ID NO: 261 - protein, 18 aa, synthetic construct
  REGION 1..18: note = Synthetic
CASSDQLRRTTRNYEQYF

SEQ ID NO: 262 - protein, 15 aa, synthetic construct
  REGION 1..15: note = Synthetic
CASSKALWVTGGYTF

SEQ ID NO: 263 - protein, 15 aa, synthetic construct
  REGION 1..15: note = Synthetic
CASSQDRVNTGELFF

SEQ ID NO: 264 - protein, 13 aa, synthetic construct
  REGION 1..13: note = Synthetic
CSARRGDNSPLHF

SEQ ID NO: 265 - protein, 11 aa, synthetic construct
  REGION 1..11: note = Synthetic
CASQTHNEQFF

SEQ ID NO: 266 - protein, 11 aa, synthetic construct
  REGION 1..11: note = Synthetic
CASSPQTEAFF

SEQ ID NO: 267 - protein, 15 aa, synthetic construct
  REGION 1..15: note = Synthetic
CASSSFRGAGETQYF

SEQ ID NO: 268 - protein, 14 aa, synthetic construct
  REGION 1..14: note = Synthetic
CSARNPLATYEQYF

SEQ ID NO: 269 - protein, 15 aa, synthetic construct
  REGION 1..15: note = Synthetic
CATSQGTRPLSAQHF

SEQ ID NO: 270 - protein, 17 aa, synthetic construct
  REGION 1..17: note = Synthetic
CASSHKGTGVLDSPLHF

SEQ ID NO: 271 - protein, 13 aa, synthetic construct
  REGION 1..13: note = Synthetic
CASSFTGGDEQFF

SEQ ID NO: 272 - protein, 16 aa, synthetic construct
  REGION 1..16: note = Synthetic
CASSLGLAGGRNIQYF

SEQ ID NO: 273 - protein, 16 aa, synthetic construct
  REGION 1..16: note = Synthetic
CSAPARGAETFYEQYF

SEQ ID NO: 274 - protein, 14 aa, synthetic construct
  REGION 1..14: note = Synthetic
CASMGDTGANVLTF

SEQ ID NO: 275 - protein, 17 aa, synthetic construct
  REGION 1..17: note = Synthetic
CASSPPRGGGNTGELFF

SEQ ID NO: 276 - protein, 13 aa, synthetic construct
  REGION 1..13: note = Synthetic
CASRRDLNYGYTF

SEQ ID NO: 277 - protein, 14 aa, synthetic construct
  REGION 1..14: note = Synthetic
CASSQRVRNSPLHF

SEQ ID NO: 278 - protein, 15 aa, synthetic construct
  REGION 1..15: note = Synthetic
CASTATGHYNSPLHF

SEQ ID NO: 279 - protein, 13 aa, synthetic construct
  REGION 1..13: note = Synthetic
CASSELRNNEQFF

SEQ ID NO: 280 - protein, 17 aa, synthetic construct
  REGION 1..17: note = Synthetic
CASSVVRTDRGRETQYF

SEQ ID NO: 281 - protein, 15 aa, synthetic construct
  REGION 1..15: note = Synthetic
CSARFVRGRNQPQHF

SEQ ID NO: 282 - protein, 14 aa, synthetic construct
  REGION 1..14: note = Synthetic
CASGTAPDRNEQFF

SEQ ID NO: 283 - protein, 13 aa, synthetic construct
  REGION 1..13: note = Synthetic
CASRAGGDETQYF

SEQ ID NO: 284 - protein, 17 aa, synthetic construct
  REGION 1..17: note = Synthetic
CASSFVTSGRVTDTQYF

SEQ ID NO: 285 - protein, 14 aa, synthetic construct
  REGION 1..14: note = Synthetic
CASSLERGWNEQFF

SEQ ID NO: 286 - protein, 19 aa, synthetic construct
  REGION 1..19: note = Synthetic
CASSSPRLAGGPSAYEQYF

SEQ ID NO: 287 - protein, 13 aa, synthetic construct
  REGION 1..13: note = Synthetic
CASSERENQPQHF

SEQ ID NO: 288 - protein, 13 aa, synthetic construct
  REGION 1..13: note = Synthetic
CASSVRQSYEQYF

SEQ ID NO: 289 - protein, 15 aa, synthetic construct
  REGION 1..15: note = Synthetic
CATRPGQSLNTEAFF

SEQ ID NO: 290 - protein, 14 aa, synthetic construct
  REGION 1..14: note = Synthetic
CASSLWGGDQPQHF

SEQ ID NO: 291 - protein, 17 aa, synthetic construct
  REGION 1..17: note = Synthetic
CASSHRGSGRAPDTQYF

SEQ ID NO: 292 - protein, 15 aa, synthetic construct
  REGION 1..15: note = Synthetic
CASRGLAGSGGELFF

SEQ ID NO: 293 - protein, 17 aa, synthetic construct
  REGION 1..17: note = Synthetic
CASSIDSTGKGPDTQYF

SEQ ID NO: 294 - protein, 13 aa, synthetic construct
  REGION 1..13: note = Synthetic
CASSPPSRYGYTF

SEQ ID NO: 295 - protein, 16 aa, synthetic construct
  REGION 1..16: note = Synthetic
CATSSSYRGRSQTQYF

SEQ ID NO: 296 - protein, 14 aa, synthetic construct
  REGION 1..14: note = Synthetic
CASSLAGLGNEQFF

SEQ ID NO: 297 - protein, 16 aa, synthetic construct
  REGION 1..16: note = Synthetic
CASSQDGLAGFLEQFF

SEQ ID NO: 298 - protein, 13 aa, synthetic construct
  REGION 1..13: note = Synthetic
CSAGGRVAETQYF

SEQ ID NO: 299 - protein, 14 aa, synthetic construct
  REGION 1..14: note = Synthetic
CASSEQGVYHEQFF

SEQ ID NO: 300 - protein, 16 aa, synthetic construct
  REGION 1..16: note = Synthetic
CAWSTPGLRSVGTQYF

SEQ ID NO: 301 - protein, 15 aa, synthetic construct
  REGION 1..15: note = Synthetic
CASSSPTGRDTEAFF

SEQ ID NO: 302 - protein, 14 aa, synthetic construct
  REGION 1..14: note = Synthetic
CAWSVQGRGNGYTF

SEQ ID NO: 303 - protein, 12 aa, synthetic construct
  REGION 1..12: note = Synthetic
CSPTSGEETQYF

SEQ ID NO: 304 - protein, 14 aa, synthetic construct
  REGION 1..14: note = Synthetic
CASSLRQGAHEQYF

SEQ ID NO: 305 - protein, 16 aa, synthetic construct
  REGION 1..16: note = Synthetic
CASGARTSGRRAVLFF

SEQ ID NO: 306 - protein, 16 aa, synthetic construct
  REGION 1..16: note = Synthetic
CASSLVLAGRNNEQFF

SEQ ID NO: 307 - protein, 14 aa, synthetic construct
  REGION 1..14: note = Synthetic
CAWEVKGPNQPQHF

SEQ ID NO: 308 - protein, 16 aa, synthetic construct
  REGION 1..16: note = Synthetic
CASSQSRQEASYEQYF

SEQ ID NO: 309 - protein, 19 aa, synthetic construct
  REGION 1..19: note = Synthetic
CASSQGGTSGRALRDTQYF

SEQ ID NO: 310 - protein, 15 aa, synthetic construct
  REGION 1..15: note = Synthetic
CASSPQRGLNTEAFF

SEQ ID NO: 311 - protein, 18 aa, synthetic construct
  REGION 1..18: note = Synthetic
CASSPAGGVAASSYEQYF

SEQ ID NO: 312 - protein, 17 aa, synthetic construct
  REGION 1..17: note = Synthetic
CASSLGNWRTPSYGYTF

SEQ ID NO: 313 - protein, 12 aa, synthetic construct
  REGION 1..12: note = Synthetic
CASSHPNYGYTF

SEQ ID NO: 314 - protein, 13 aa, synthetic construct
  REGION 1..13: note = Synthetic
CASSRTGNQPQHF

SEQ ID NO: 315 - protein, 14 aa, synthetic construct
  REGION 1..14: note = Synthetic
CSAREGGFGNEQYF

SEQ ID NO: 316 - protein, 13 aa, synthetic construct
  REGION 1..13: note = Synthetic
CASAQGGDTEAFF

SEQ ID NO: 317 - protein, 13 aa, synthetic construct
  REGION 1..13: note = Synthetic
CASSPLQTGELFF

SEQ ID NO: 318 - protein, 11 aa, synthetic construct
  REGION 1..11: note = Synthetic
CSAPGGYGYTF

SEQ ID NO: 319 - protein, 13 aa, synthetic construct
  REGION 1..13: note = Synthetic
CSAKDRGKRGYTF

SEQ ID NO: 320 - protein, 14 aa, synthetic construct
  REGION 1..14: note = Synthetic
CAWTYRTGGVEQYF

SEQ ID NO: 321 - protein, 15 aa, synthetic construct
  REGION 1..15: note = Synthetic
CASSLDSKAKNIQYF

SEQ ID NO: 322 - protein, 17 aa, synthetic construct
  REGION 1..17: note = Synthetic
CASGLRTSGKLRNEQFF

SEQ ID NO: 323 - protein, 14 aa, synthetic construct
  REGION 1..14: note = Synthetic
CASSLGASGTEQFF

SEQ ID NO: 324 - protein, 15 aa, synthetic construct
  REGION 1..15: note = Synthetic
CASSSDRGLHEKLFF

SEQ ID NO: 325 - protein, 17 aa, synthetic construct
  REGION 1..17: note = Synthetic
CASSLRGGGNGWDTQYF

SEQ ID NO: 326 - protein, 14 aa, synthetic construct
  REGION 1..14: note = Synthetic
CATRGGPDEETQYF

SEQ ID NO: 327 - protein, 15 aa, synthetic construct
  REGION 1..15: note = Synthetic
CASSSPTGGDYEQYF

SEQ ID NO: 328 - protein, 14 aa, synthetic construct
  REGION 1..14: note = Synthetic
CASSLSGSNEKLFF

SEQ ID NO: 329 - protein, 16 aa, synthetic construct
  REGION 1..16: note = Synthetic
CASSLRRPGQGWELFF

SEQ ID NO: 330 - protein, 15 aa, synthetic construct
  REGION 1..15: note = Synthetic
CASSLATGGLSPLHF

SEQ ID NO: 331 - protein, 10 aa, synthetic construct
  REGION 1..10: note = Synthetic
CASRDDGYTF

SEQ ID NO: 332 - protein, 18 aa, synthetic construct
  REGION 1..18: note = Synthetic
CASSSPGLAGGNTGELFF

SEQ ID NO: 333 - protein, 18 aa, synthetic construct
  REGION 1..18: note = Synthetic
CASSYSPAIGGVYNEQFF

SEQ ID NO: 334 - protein, 16 aa, synthetic construct
  REGION 1..16: note = Synthetic
CSGKGLAGGAQETQYF

SEQ ID NO: 335 - protein, 13 aa, synthetic construct
  REGION 1..13: note = Synthetic
CASTQGGVETQYF

SEQ ID NO: 336 - protein, 15 aa, synthetic construct
  REGION 1..15: note = Synthetic
CASSQEQGRNSPLHF

SEQ ID NO: 337 - protein, 13 aa, synthetic construct
  REGION 1..13: note = Synthetic
CASSQYNHNEQFF

SEQ ID NO: 338 - protein, 14 aa, synthetic construct
  REGION 1..14: note = Synthetic
CASGLAPLSREQYF

SEQ ID NO: 339 - protein, 15 aa, synthetic construct
  REGION 1..15: note = Synthetic
CASSLGGSSNQPQHF

SEQ ID NO: 340 - protein, 15 aa, synthetic construct
  REGION 1..15: note = Synthetic
CASSPWPQDGEKLFF

SEQ ID NO: 341 - protein, 15 aa, synthetic construct
  REGION 1..15: note = Synthetic
CSARKVQRGKNIQYF

SEQ ID NO: 342 - protein, 14 aa, synthetic construct
  REGION 1..14: note = Synthetic
CSARADGQGYGYTF

SEQ ID NO: 343 - protein, 15 aa, synthetic construct
  REGION 1..15: note = Synthetic
CASSQEVGGDHGYTF

SEQ ID NO: 344 - protein, 14 aa, synthetic construct
  REGION 1..14: note = Synthetic
CASIRPGLNYGYTF

SEQ ID NO: 345 - protein, 14 aa, synthetic construct
  REGION 1..14: note = Synthetic
CASSPADREETQYF

SEQ ID NO: 346 - protein, 15 aa, synthetic construct
  REGION 1..15: note = Synthetic
CATSEPGPLNQPQHF

SEQ ID NO: 347 - protein, 15 aa, synthetic construct
  REGION 1..15: note = Synthetic
CASRTTTGFLSPLHF

SEQ ID NO: 348 - protein, 13 aa, synthetic construct
  REGION 1..13: note = Synthetic
CSGLAGGQETQYF

SEQ ID NO: 349 - protein, 15 aa, synthetic construct
  REGION 1..15: note = Synthetic
CASSKPGQGWETQYF

SEQ ID NO: 350 - protein, 14 aa, synthetic construct
  REGION 1..14: note = Synthetic
CATSSSGFSYEQYF

SEQ ID NO: 351 - protein, 17 aa, synthetic construct
  REGION 1..17: note = Synthetic
CASSYIVGLTTNEKLFF

SEQ ID NO: 352 - protein, 13 aa, synthetic construct
  REGION 1..13: note = Synthetic
CAWGTLWSGELFF

SEQ ID NO: 353 - protein, 16 aa, synthetic construct
  REGION 1..16: note = Synthetic
CASPPVSGTDYNEQFF

SEQ ID NO: 354 - protein, 15 aa, synthetic construct
  REGION 1..15: note = Synthetic
CASSLYDRPRYGYTF

SEQ ID NO: 355 - protein, 13 aa, synthetic construct
  REGION 1..13: note = Synthetic
CASYGQGVHEQFF

SEQ ID NO: 356 - protein, 17 aa, synthetic construct
  REGION 1..17: note = Synthetic
CASSPPIGFTTNEKLFF

SEQ ID NO: 357 - protein, 15 aa, synthetic construct
  REGION 1..15: note = Synthetic
DKRLAAYLMLMRSPS

SEQ ID NO: 358 - protein, 15 aa, synthetic construct
  REGION 1..15: note = Synthetic
TLTAFGFASADLIEI

SEQ ID NO: 359 - protein, 15 aa, synthetic construct
  REGION 1..15: note = Synthetic
FLHYIFMENAFELPT

SEQ ID NO: 360 - protein, 15 aa, synthetic construct
  REGION 1..15: note = Synthetic
VEFVTNMGIIIPDFA

SEQ ID NO: 361 - protein, 15 aa, synthetic construct
  REGION 1..15: note = Synthetic
VGSKLIVAMSSWLQK

SEQ ID NO: 362 - protein, 15 aa, synthetic construct
  REGION 1..15: note = Synthetic
IKHIYAISSAALSAS

SEQ ID NO: 363 - protein, 15 aa, synthetic construct
  REGION 1..15: note = Synthetic
HFSNVFRSVMAPFTM

SEQ ID NO: 364 - protein, 15 aa, synthetic construct
  REGION 1..15: note = Synthetic
QLYSKFLLKAEPLAF

SEQ ID NO: 365 - protein, 15 aa, synthetic construct
  REGION 1..15: note = Synthetic
LSQLQTYMIQFDQYI

SEQ ID NO: 366 - protein, 15 aa, synthetic construct
  REGION 1..15: note = Synthetic
HVKHFVINLIGDFEV

SEQ ID NO: 367 - protein, 15 aa, synthetic construct
  REGION 1..15: note = Synthetic
LIINWLQEALSSASL

SEQ ID NO: 368 - protein, 15 aa, synthetic construct
  REGION 1..15: note = Synthetic
LEVLNFDFQANAQLS

SEQ ID NO: 369 - protein, 15 aa, synthetic construct
  REGION 1..15: note = Synthetic
SLFFSAQPFEITAST

SEQ ID NO: 370 - protein, 15 aa, synthetic construct
  REGION 1..15: note = Synthetic
GKIDFLNNYALFLSP

SEQ ID NO: 371 - protein, 15 aa, synthetic construct
  REGION 1..15: note = Synthetic
RGLKLATALSLSNKF

SEQ ID NO: 372 - protein, 15 aa, synthetic construct
  REGION 1..15: note = Synthetic
ILFSYFQDLVITLPF

SEQ ID NO: 373 - protein, 15 aa, synthetic construct
  REGION 1..15: note = Synthetic
KFTYLINYIQDEINT

SEQ ID NO: 374 - protein, 15 aa, synthetic construct
  REGION 1..15: note = Synthetic
QIHQYIMALREEYFD

SEQ ID NO: 375 - protein, 15 aa, synthetic construct
  REGION 1..15: note = Synthetic
KIVSLIKNLLVALKD

SEQ ID NO: 376 - protein, 15 aa, synthetic construct
  REGION 1..15: note = Synthetic
YKKLRTSSFALNLPT